SEQUENCE LISTING (138 sequences)

SEQ ID NO: 1 - DNA, 673 bp, Homo sapiens
atgcttcctcctgccattcatttctatctccttccccttgcatgcatcctaatgaaaagctgtttggcttttaaaaatgatgccacagaaatcctttattcacatgtggttaaacctgttccagcacaccccagcagcaacagcacgttgaatcaagccagaaatggaggcaggcatttcagtaacactggactggatcggaacactcgggttcaagtgggttgccgggaactgcgttccaccaaatacatctctgatggccagtgcaccagcatcagccctctgaaggagctggtgtgtgctggcgagtgcttgcccctgccagtgctccctaactggattggaggaggctatggaacaaagtactggagcaggaggagctcccaggagtggcggtgtgtcaatgacaaaacccgtacccagagaatccagctgcagtgccaagatggcagcacacgcacctacaaaatcacagtagtcactgcctgcaagtgcaagaggtacacccggcagcacaacgagtccagtcacaactttgagagcatgtcacctgccaagccagtccagcatcacagagagcggaaaagagccagcaaatccagcaagcacagcatgagttagctcgaggggcggatcccccgggctgcaggaattcgatatcaagcttgctagc

SEQ ID NO: 2 - protein, 206 aa, Homo sapiens
MLPPAIHFYLLPLACILMKSCLAFKNDATEILYSHVVKPVPAHPSSNSTLNQARNGGRHFSNTGLDRNTRVQVGCRELRSTKYISDGQCTSISPLKELVCAGECLPLPVLPNWIGGGYGTKYWSRRSSQEWRCVNDKTRTQRIQLQCQDGSTRTYKITVVTACKCKRYTRQHNESSHNFESMSPAKPVQHHRERKRASKSSKHSMS

SEQ ID NO: 3 - DNA, 627 bp, Mus musculus
atgcttcctcctgccattcatctctctctcattcccctgctctgcatcctgatgagaaactgtttggcttttaaaaatgatgccacagaaatcctttattcacatgtggttaaacctgtcccggcacaccccagcagcaacagcaccctgaatcaagccaggaatggaggcaggcatttcagtagcactggactggatcgaaacagtcgagttcaagtgggctgcagggaactgcggtccaccaaatacatttcggacggccagtgcaccagcatcagccctctgaaggagctggtgtgcgcgggcgagtgcttgcccctgccggtgcttcccaactggatcggaggaggctatggaacaaagtactggagccggaggagctctcaggagtggcggtgtgtcaacgacaagacgcgcacccagaggatccagctgcagtgtcaggacggcagcacgcgcacctacaaaatcaccgtggtcacggcgtgcaagtgcaagaggtacacccgtcagcacaacgagtccagccacaactttgaaagcgtgtcgcccgccaagcccgcccagcaccacagagagcggaagagagccagcaaatccagcaagcacagtctgagctagctcgag

SEQ ID NO: 4 - protein, 206 aa, Mus musculus
MLPPAIHLSLIPLLCILMRNCLAFKNDATEILYSHVVKPVPAHPSSNSTLNQARNGGRHFSSTGLDRNSRVQVGCRELRSTKYISDGQCTSISPLKELVCAGECLPLPVLPNWIGGGYGTKYWSRRSSQEWRCVNDKTRTQRIQLQCQDGSTRTYKITVVTACKCKRYTRQHNESSHNFESVSPAKPAQHHRERKRASKSSKHSLS

SEQ ID NO: 5 - DNA, 629 bp, Rattus norvegicus
atgcttcctcctgccattcatctctctctcattcccctgctctgcatcctgatgaaaaactgtttggcttttaaaaatgatgccacagaaatcctttattcacatgtggttaaacctgtttcagcacaccccagcagcaacagcaccttgaatcaagccaggaatggaggcaggcacttcagtagcacgggactggatcgaaatagtcgagttcaagtgggctgcagggaactgcggtccaccaaatacatctcggatggccagtgcaccagcatcagccctctgaaggagctggtgtgcgcgggtgagtgcttgcccttgccagtgcttcccaactggatcggaggaggctacggaacaaagtactggagccggaggagctcccaggagtggcggtgtgtcaacgacaagacgcgcacccagagaatccagctgcagtgtcaggacggcagcacacgcacctacaaaatcaccgtggtcacagcgtgcaagtgcaagaggtacacccggcagcacaacgagtccagccacaactttgaaagcgtgtctcccgccaagcccgcccagcaccacagagagcggaagagagccagcaaatccagcaagcacagtctgagctaggcggccgc

SEQ ID NO: 6 - protein, 206 aa, Rattus norvegicus
MLPPAIHFYLLPLACILMKSCLAFKNDATEILYSHVVKPVPAHPSSNSTMNQARNGGRHFSNTGLDRNTRVQVGCRELRSTKYISDGQCTSISPLKELVCAGECLPLPVLPNWIGGGYGTKYWSRRSSQEWRCVNDKTRTQRIQLQCQDGSTRTYKITVVTACKCKRYTRQHNESSHNFESMSPAKPVQHHRERKRASKSSKHSMS

SEQ ID NO: 7 - DNA, 629 bp, Macaca fascicularis; identical to SEQ ID NO: 5

SEQ ID NO: 8 - protein, 206 aa, Macaca fascicularis
MLPPAIHLSLIPLLCILMKNCLAFKNDATEILYSHVVKPVSAHPSSNSTLNQARNGGRHFSSTGLDRNSRVQVGCRELRSTKYISDGQCTSISPLKELVCAGECLPLPVLPNWIGGGYGTKYWSRRSSQEWRCVNDKTRTQRIQLQCQDGSTRTYKITVVTACKCKRYTRQHNESSHNFESVSPAKPAQHHRERKRASKSSKHSLS

SEQ ID NO: 9 - protein, 28 aa, Homo sapiens
LPLPVLPNWIGGGYGTKYWSRRSSQEWR

SEQ ID NO: 10 - DNA, 336 bp, Mus musculus
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactaggcaatctggggtccctgatcgcttcacaggcagtggatctgggacagatttcactctcaccatcagcagtgtgcaggctgaagacctggcagtttattactgcaagcaatcttataatctcctcacgttcggtgctgggaccaagctggagctgaaa

SEQ ID NO: 11 - protein, 112 aa, Mus musculus
DIVMSQSPSSLAVSAGEKVTMSCKSSQSLLNSRTRKNYLAWYQQKPGQSPKLLIYWASTRQSGVPDRFTGSGSGTDFTLTISSVQAEDLAVYYCKQSYNLLTFGAGTKLELK

SEQ ID NO: 12 - DNA, 363 bp, Mus musculus
gaggttcagctgcagcagtctggggcagagcttgtgaggtcaggggcctcagtcaagttgtcctgcacagcttctggcttcaacattaaagactactatatacactggatgaagcagaggcctgaacagggcctcgagtggattggatggattgatcctgagaatggtgatactgaatctgccccgaagttccagggcaaggccactatgactgcagacacatcctccaacacagcctacctgcacctcagcagcctgacatttgaggacactgccgtctattactgtaatgcagaaggttacggtagtaggcactggtacttcgatgtctggggcgcagggaccacggtcaccgtctcctca

SEQ ID NO: 13 - protein, 121 aa, Mus musculus
EVQLQQSGAELVRSGASVKLSCTASGFNIKDYYIHWMKQRPEQGLEWIGWIDPENGDTESAPKFQGKATMTADTSSNTAYLHLSSLTFEDTAVYYCNAEGYGSRHWYFDVWGAGTTVTVSS

SEQ ID NO: 14 - DNA, 321 bp, Mus musculus
gacatccagatgactcagtctccagcctccctggctgcatctgtgggagaaaccatcaccatcacatgtcaagcaagtgagaacatttacttcagtttagcatggtatcagcagaagcaagggaaatctcctcagctcctgatctataatgcaaacaacttggaagatggtgtcccatcgaggttcagtggcagtggatctgggacacagtattctatgaagatcaacaacatgcagcctgaagatactgcaacttatttctgtaaagaggcttatgactctccattcacgttcggcacggggacaaaattggaaataaaa

SEQ ID NO: 15 - protein, 107 aa, Mus musculus
DIQMTQSPASLAASVGETITITCQASENIYFSLAWYQQKQGKSPQLLIYNANNLEDGVPSRFSGSGSGTQYSMKINNMQPEDTATYFCKEAYDSPFTFGTGTKLEIK

SEQ ID NO: 16 - DNA, 357 bp, Mus musculus
gaaattcaactccagcagtctgggactgtgctgacaaggcctggggcttcagtgaagatgtcctgcaagacttctggctacacctttaccagctactggatgcactgggtaaaacagaggcctggacagggtctggaatggattggcgctctttatcctggaaatagtgttactaactacaaccagaagttcaagggcaaggccaaactgactgcagtcacatccaccagcactgcctacatggagctcagcagcctgacaaatgaggactctgcggtctattactgtacaagaggatttcttactgcgccctactttgactcctggggccaaggcaccactctcacagtctcctca

SEQ ID NO: 17 - protein, 119 aa, Mus musculus
EIQLQQSGTVLTRPGASVKMSCKTSGYTFTSYWMHWVKQRPGQGLEWIGALYPGNSVTNYNQKFKGKAKLTAVTSTSTAYMELSSLTNEDSAVYYCTRGFLTAPYFDSWGQGTTLTVSS

SEQ ID NO: 18 - DNA, 336 bp, Mus musculus
gacattgtggtgtcacaggctccatcctcccttgctgtgtcagttggagagaagattattatgagctgcaagtccagtcagagccttttacacagcagcaatcgaaggaactacttggcctggtaccaacagaaaccagggcagtctcctaaattgctgatttcctgggcatccattagggaatctggggtccctgatcgcttcacaggcagtggatccgggacagatttcactctcaccatcagcagcgtgaagactgaagacctggcaatttattactgtcaccaatattatacttattccacgttcggtgctgggaccaagctggagctgaag

SEQ ID NO: 19 - protein, 112 aa, Mus musculus
DIVVSQAPSSLAVSVGEKIIMSCKSSQSLLHSSNRRNYLAWYQQKPGQSPKLLISWASIRESGVPDRFTGSGSGTDFTLTISSVKTEDLAIYYCHQYYTYSTFGAGTKLELK

SEQ ID NO: 20 - DNA, 369 bp, Mus musculus
caggttactctaaaagagtctggccctgagatactgcagccctcccagaccctcagtctgacttgttcgttctctgggttttcactgaccacttctggtatgggtgtgagctggattcgtcagccttcaggagggagtctggaatggctggctcacattttctgggatgatgacaagcggtataatccatccctgacgagtcgactcacaatctccaaggatgcccccagaaaccaggttttcctcaaaatcaccagtgtggacactgcagatgctgccacatattactgtgctcgaggaggagattattacagtactggatttggctttgattactggggccaagggactctggtcactgtctctgca

SEQ ID NO: 21 - protein, 123 aa, Mus musculus
QVTLKESGPEILQPSQTLSLTCSFSGFSLTTSGMGVSWIRQPSGGSLEWLAHIFWDDDKRYNPSLTSRLTISKDAPRNQVFLKITSVDTADAATYYCARGGDYYSTGFGFDYWGQGTLVTVSA

SEQ ID NO: 22 - DNA, 321 bp, Mus musculus
gacatccagatgactcagtctccagcctccctggctgcatctgtgggagaatccatcaccatcacatgtcaggcaagtgagaacatttacttcagtttagcatggtatcagcagaagcaagggaggtctcctcagctcctgatctatcatgcaaaaagtttggaagatggtgtcccatcgaggttcagtggcagtggctctgggacacagtattctatgaagatcaacagcatgcagcctgaagatactgcaacttatttctgtaaacaggcttatgaccatccattcacgttcggcacggggacaaaattggaaatgaaa

SEQ ID NO: 23 - protein, 107 aa, Mus musculus
DIQMTQSPASLAASVGESITITCQASENIYFSLAWYQQKQGRSPQLLIYHAKSLEDGVPSRFSGSGSGTQYSMKINSMQPEDTATYFCKQAYDHPFTFGTGTKLEMK

SEQ ID NO: 24 - DNA, 357 bp, Mus musculus
gaggttcagctccagcagtctgggactgtgctggcaaggcctggggcttcagtgaagatgtcctgtaaggcttctggctacacctttaccagctactggatgcactgggtaaaacagaggcctggacagggtctggaatggattgtcgctatttatcctggaaatagtgatactaactacaaccagaagttcaagggcaaggccaaactgactgcagtcacatccaccagcactgcctacatggaactcaacagcctgacaaatgaggactctgcggtctattactgtgtaagaggatttattactgcgccctactttgactactggggccaaggcaccactctcacagtctcctca

SEQ ID NO: 25 - protein, 119 aa, Mus musculus
EVQLQQSGTVLARPGASVKMSCKASGYTFTSYWMHWVKQRPGQGLEWIVAIYPGNSDTNYNQKFKGKAKLTAVTSTSTAYMELNSLTNEDSAVYYCVRGFITAPYFDYWGQGTTLTVSS

SEQ ID NO: 26 - DNA, 336 bp, Mus musculus
gacattgtgatgtcacagtctccatcctccctggctgtgtcagcaggagagaaggtcactatgagctgcaaatccagtcagagtctgctcaacagtagaacccgaaagaactacttggcttggtaccagcagaagccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatcttccgacgttcggtggaggcaccaggctggaaatcaaa

SEQ ID NO: 27 - protein, 112 aa, Mus musculus
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLPTFGGGTRLEIK

SEQ ID NO: 28 - DNA, 360 bp, Mus musculus
gaggttcagctgcagcagtctggggcagagcttgtgaggtcaggggcctcagtcaagttgtcctgcacagcttctggcttcaacattaaagactactatatgcactgggtgaagcagaggcctgaacagggcctggagtggattggatggattgatcctgaaaatggtgatactgaatatgccccgaagttccagggcaaggccactatgactgcagacacatcctccaacacagcctacctgcagctcagcagcctgacatctgaggacactgccgtcttttactgtaatttctatgatgtttactccgaggggactatggcctactggggtcaaggaacctcagtcaccgtctcctca

SEQ ID NO: 29 - protein, 120 aa, Mus musculus
EVQLQQSGAELVRSGASVKLSCTASGFNIKDYYMHWVKQRPEQGLEWIGWIDPENGDTEYAPKFQGKATMTADTSSNTAYLQLSSLTSEDTAVFYCNFYDVYSEGTMAYWGQGTSVTVSS

SEQ ID NO: 30 - DNA, 321 bp, Mus musculus
gacatccagatgactcagtctccagcttcactgtctgcatctgtgggagaaactgtcaccatcacatgtggagcaagtgagaatatttacggtgctttaaattggtatcagcggaaacagggaaaatctcctcaggtcctgatctatggtgcaaccaacttggcagatggcatgtcatcgaggttcagtggcagtggatctggtagacagtattctctcaagatcagtagcctgcatcctgacgatgttgcaatgtattactgtcaaaatgtgttcagtagtccgctcacgttcggtgctgggaccaagctggagctgaaa

SEQ ID NO: 31 - protein, 107 aa, Mus musculus
DIQMTQSPASLSASVGETVTITCGASENIYGALNWYQRKQGKSPQVLIYGATNLADGMSSRFSGSGSGRQYSLKISSLHPDDVAMYYCQNVFSSPLTFGAGTKLELK

SEQ ID NO: 32 - DNA, 336 bp, Mus musculus
cagatccagttggtgcagtctggacctgagctgaagaagcctggagagacagtcaagatctcctgcaaggcttctggttataccttcacagactattcaatgcactgggtgaagcaggctccaggaaagggtttaaagtggatgggctggataaacactgagactggtgagccaacatatgcagatgacttcaagggacggtttgccttctctttggaaacctctgccagcactgcctgtttggagatcaacaacctcaaaaatgaggacacggctacatatttctgttctttaactgggtactggggtcaaggaacctcagtcaccgtctcctca

SEQ ID NO: 33 - protein, 112 aa, Mus musculus
QIQLVQSGPELKKPGETVKISCKASGYTFTDYSMHWVKQAPGKGLKWMGWINTETGEPTYADDFKGRFAFSLETSASTACLEINNLKNEDTATYFCSLTGYWGQGTSVTVSS

SEQ ID NO: 34 - protein, 17 aa, Mus musculus
KSSQSLLNSRTRKNYLA

SEQ ID NO: 35 - protein, 7 aa, Mus musculus
WASTRQS

SEQ ID NO: 36 - protein, 8 aa, Mus musculus
KQSYNLLT

SEQ ID NO: 37 - protein, 5 aa, Mus musculus
DYYIH

SEQ ID NO: 38 - protein, 17 aa, Mus musculus
WIDPENGDTESAPKFQG

SEQ ID NO: 39 - protein, 12 aa, Mus musculus
EGYGSRHWYFDV

SEQ ID NO: 40 - protein, 11 aa, Mus musculus
QASENIYFSLA

SEQ ID NO: 41 - protein, 7 aa, Mus musculus
NANNLED

SEQ ID NO: 42 - protein, 9 aa, Mus musculus
KEAYDSPFT

SEQ ID NO: 43 - protein, 5 aa, Mus musculus
SYWMH

SEQ ID NO: 44 - protein, 17 aa, Mus musculus
ALYPGNSVTNYNQKFKG

SEQ ID NO: 45 - protein, 10 aa, Mus musculus
GFLTAPYFDS

SEQ ID NO: 46 - protein, 17 aa, Mus musculus
KSSQSLLHSSNRRNYLA

SEQ ID NO: 47 - protein, 7 aa, Mus musculus
WASIRES

SEQ ID NO: 48 - protein, 8 aa, Mus musculus
HQYYTYST

SEQ ID NO: 49 - protein, 7 aa, Mus musculus
TSGMGVS

SEQ ID NO: 50 - protein, 16 aa, Mus musculus
HIFWDDDKRYNPSLTS

SEQ ID NO: 51 - protein, 13 aa, Mus musculus
GGDYYSTGFGFDY

SEQ ID NO: 52 - protein, 11 aa, Mus musculus
QASENIYFSLA

SEQ ID NO: 53 - protein, 7 aa, Mus musculus
HAKSLED

SEQ ID NO: 54 - protein, 9 aa, Mus musculus
KQAYDHPFT

SEQ ID NO: 55 - protein, 5 aa, Mus musculus
SYWMH

SEQ ID NO: 56 - protein, 17 aa, Mus musculus
AIYPGNSDTNYNQKFKG

SEQ ID NO: 57 - protein, 10 aa, Mus musculus
GFITAPYFDY

SEQ ID NO: 58 - protein, 17 aa, Mus musculus
KSSQSLLNSRTRKNYLA

SEQ ID NO: 59 - protein, 7 aa, Mus musculus
WASTRES

SEQ ID NO: 60 - protein, 8 aa, Mus musculus
KQSYNLPT

SEQ ID NO: 61 - protein, 5 aa, Mus musculus
DYYMH

SEQ ID NO: 62 - protein, 17 aa, Mus musculus
WIDPENGDTEYAPKFQG

SEQ ID NO: 63 - protein, 11 aa, Mus musculus
YDVYSEGTMAY

SEQ ID NO: 64 - protein, 11 aa, Mus musculus
GASENIYGALN

SEQ ID NO: 65 - protein, 7 aa, Mus musculus
GATNLAD

SEQ ID NO: 66 - protein, 9 aa, Mus musculus
QNVFSSPLT

SEQ ID NO: 67 - protein, 5 aa, Mus musculus
DYSMH

SEQ ID NO: 68 - protein, 17 aa, Mus musculus
WINTETGEPTYADDFKG

SEQ ID NO: 69 - protein, 3 aa, Mus musculus
TGY

SEQ ID NO: 70 - DNA, 321 bp, Homo sapiens
gacatccagatgactcagtctccagcttcactgtctgcatctgtgggagaaactgtcaccatcacatgtggagccagtgagaatatttacggtgctttaaattggtatcagcggaaacagggaaaatctcctcagctcctgatctttggtgcaaccaacttggcagatggcatgtcatcgaggttcagtggcagtggatctggtagacagtattctctcgagatcagtagcctgcatcctgacgatgttgcaacgtattactgtcaaaatttatttaattctccgctcacattcggtgctgggaccaagctggacctgaaa

SEQ ID NO: 71 - protein, 107 aa, Homo sapiens
DIQMTQSPASLSASVGETVTITCGASENIYGALNWYQRKQGKSPQLLIFGATNLADGMSSRFSGSGSGRQYSLEISSLHPDDVATYYCQNLFNSPLTFGAGTKLDLK

SEQ ID NO: 72 - DNA, 336 bp, Homo sapiens
cagatccagttggtgcagtctggacctgagctgaagaagcctggagagacagtcaagatctcctgcaaggcttctggttataccttcacagactattcaatgcactgggtgaagcaggctccaggaaagggtttaaagtggatgggctggataaacactgagactggtgagccaacatatgcagctgacttcaagggacggtttgccttctctttggaaacctctgccagcactgcctatttgcagatcaacaacctcaaaaatgaggacacggctacatatttctgtactttaactgggtactggggtcagggaacctcagtcaccgtctcctca

SEQ ID NO: 73 - protein, 112 aa, Homo sapiens
QIQLVQSGPELKKPGETVKISCKASGYTFTDYSMHWVKQAPGKGLKWMGWINTETGEPTYAADFKGRFAFSLETSASTAYLQINNLKNEDTATYFCTLTGYWGQGTSVTVSS

SEQ ID NO: 74 - DNA, 336 bp, Homo sapiens
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactaggaaatctggggtccctgatcgcttcataggcagtggatctgggacagatttcactctcaccattagcagtgtgcaggctgaagacctggcagtttattactgcaagcaatcttataatctcgtcacgttcggtgctgggaccaagctggagctgaaa

SEQ ID NO: 75 - protein, 112 aa, Homo sapiens
DIVMSQSPSSLAVSAGEKVTMSCKSSQSLLNSRTRKNYLAWYQQKPGQSPKLLIYWASTRKSGVPDRFIGSGSGTDFTLTISSVQAEDLAVYYCKQSYNLVTFGAGTKLELK

SEQ ID NO: 76 - DNA, 363 bp, Homo sapiens
gaggttcagctgcagcagtctggggcagagcttgtgaggtcaggggcctcagtcaggttgtcctgcacagcttctggcttcaacattaaagacttctatatgcactggttgaagcagaggcctgaacagggcctggagtggattggatggattgatcctgagaatggtgatactgagtctgccccgaagttccagggcaaggccactatgactgcagacacatcctccaacacagcctacctgcagctcagcagcctgacatctgaggacactgccgtctattgctgtaatgcagaaggctacgataatagccactggtacttcgatgtctggggcgcagggaccacggtcaccgtctcctca

SEQ ID NO: 77 - protein, 121 aa, Homo sapiens
EVQLQQSGAELVRSGASVRLSCTASGFNIKDFYMHWLKQRPEQGLEWIGWIDPENGDTESAPKFQGKATMTADTSSNTAYLQLSSLTSEDTAVYCCNAEGYDNSHWYFDVWGAGTTVTVSS

SEQ ID NO: 78 - DNA, 336 bp, Homo sapiens
gacattgtggtgtcacaggctccatcctcccttgctgtgtcagttggagagaagattattatgagctgcaagtccagtcagagccttttacacagcagcaatcaaaggaactacttggcctggtaccaacagaaaccagggcagtctcctaaactgctgatttcctgggcatccattagggaatctggggtccctgatcgcttcacaggcagtggatctgggacagatttcactctcaccatcagcagcgtgaagactgaagacctggcagtttattattgtcaccaatattatagttattccacgttcggtgctgggaccaagctggagctgaag

SEQ ID NO: 79 - protein, 112 aa, Homo sapiens
DIVVSQAPSSLAVSVGEKIIMSCKSSQSLLHSSNQRNYLAWYQQKPGQSPKLLISWASIRESGVPDRFTGSGSGTDFTLTISSVKTEDLAVYYCHQYYSYSTFGAGTKLELK

SEQ ID NO: 80 - DNA, 369 bp, Homo sapiens
caggttactctaaaagagtctggccctgggatattgcagccctcccagaccctcagtctgacttgttctttctctgggttttcactgaccacttctggtatgggtgtgagctggattcgtcagccttcaggagggagtctggaatggctggcacacattttctgggatgatgacaagcgctataatccatccctgacgagccgactcacaatctccaaggatgcctccagaaaccaggttttcctcaagatcagcagtgtggacactgcagacgctgccacatactactgtgctcgaggaggagattactacagtactggatttggctttgattactggggccaagggactctggtcactgtctctgca

SEQ ID NO: 81 - protein, 123 aa, Homo sapiens
QVTLKESGPGILQPSQTLSLTCSFSGFSLTTSGMGVSWIRQPSGGSLEWLAHIFWDDDKRYNPSLTSRLTISKDASRNQVFLKISSVDTADAATYYCARGGDYYSTGFGFDYWGQGTLVTVSA

SEQ ID NO: 82 - DNA, 336 bp, Homo sapiens
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattattgcaagcaatcttataatcttccgacgttcggtggaggcaccaagctggaaatcaaa

SEQ ID NO: 83 - protein, 112 aa, Homo sapiens
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLPTFGGGTKLEIK

SEQ ID NO: 84 - DNA, 360 bp, Homo sapiens
gaggttcagctgcagcagtctggggcagagcttgtgaggtcaggggcctcagtcaagttgtcctgcacagcttctggcttcaacattaaagactactatatgcattgggtgaagcagaggcctgaacagggcctggagtggattggatggattgatcctgagaatggtgatactgaatatgccccgaagttccagggcaaggccactatgactgcagacacatcctccaacacagcctacctgcagctcagcagcctgacatctgaggacactgccgtctattactgtaatttctatgatgtttactccgagggggctttggactactggggtcaaggaacctcagtcaccgtctcctca

SEQ ID NO: 85 - protein, 120 aa, Homo sapiens
EVQLQQSGAELVRSGASVKLSCTASGFNIKDYYMHWVKQRPEQGLEWIGWIDPENGDTEYAPKFQGKATMTADTSSNTAYLQLSSLTSEDTAVYYCNFYDVYSEGALDYWGQGTSVTVSS

SEQ ID NO: 86 - protein, 11 aa, Homo sapiens
GASENIYGALN

SEQ ID NO: 87 - protein, 7 aa, Homo sapiens
GATNLAD

SEQ ID NO: 88 - protein, 9 aa, Homo sapiens
QNLFNSPLT

SEQ ID NO: 89 - protein, 5 aa, Homo sapiens
DYSMH

SEQ ID NO: 90 - protein, 17 aa, Homo sapiens
WINTETGEPTYAADFKG

SEQ ID NO: 91 - protein, 17 aa, Homo sapiens
KSSQSLLNSRTRKNYLA

SEQ ID NO: 92 - protein, 7 aa, Homo sapiens
WASTRKS

SEQ ID NO: 93 - protein, 8 aa, Homo sapiens
KQSYNLVT

SEQ ID NO: 94 - protein, 5 aa, Homo sapiens
DFYMH

SEQ ID NO: 95 - protein, 17 aa, Homo sapiens
WIDPENGDTESAPKFQG

SEQ ID NO: 96 - protein, 12 aa, Homo sapiens
EGYDNSHWYFDV

SEQ ID NO: 97 - protein, 17 aa, Homo sapiens
KSSQSLLHSSNQRNYLA

SEQ ID NO: 98 - protein, 7 aa, Homo sapiens
WASIRES

SEQ ID NO: 99 - protein, 8 aa, Homo sapiens
HQYYSYST

SEQ ID NO: 100 - protein, 7 aa, Homo sapiens
TSGMGVS

SEQ ID NO: 101 - protein, 16 aa, Homo sapiens
HIFWDDDKRYNPSLTS

SEQ ID NO: 102 - protein, 13 aa, Homo sapiens
GGDYYSTGFGFDY

SEQ ID NO: 103 - protein, 17 aa, Homo sapiens
KSSQSLLNSRTRKNYLA

SEQ ID NO: 104 - protein, 7 aa, Homo sapiens
WASTRES

SEQ ID NO: 105 - protein, 8 aa, Homo sapiens
KQSYNLPT

SEQ ID NO: 106 - protein, 5 aa, Homo sapiens
DYYMH

SEQ ID NO: 107 - protein, 17 aa, Homo sapiens
WIDPENGDTEYAPKFQG

SEQ ID NO: 108 - protein, 11 aa, Homo sapiens
YDVYSEGALDY

SEQ ID NO: 109 - DNA, 336 bp, Homo sapiens
gatatcgtaatgacccagtcgcctgactcacttgcggtgtccctcggggaaagagctacaatcaattgcaagtcaagccagtccttgctcaacagcaggacgcgaaagaactacttggcgtggtaccagcaaaagccgggacaaccgcccaagttgctgatctattgggcctcaacgcgcgagtcgggggtcccagaccggttctcgggttcgggatccgggactgacttcacgctgactatttcgtcgttgcaggcagaggatgtcgcggtgtattactgtaaacagagctataaccttcccacctttggtggcggaacaaaagtggaaatcaaa

SEQ ID NO: 110 - protein, 112 aa, Homo sapiens
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLPTFGGGTKVEIK

SEQ ID NO: 111 - DNA, 360 bp, Homo sapiens
caggtacaactcgtgcagagcggagccgaagtcaaaaagcccggtgcgtcagtgaaggtatcgtgtaaggcatcagggtttaacatcaaagattactacatgcactgggtgaggcaagctccgggccaggggctggagtggatggggtggattgatccagaaaatggagacactgagtatgcacctaagttccaggggagagtgacgatgacagcggacacctcgacgtccacagtgtacatggagctgtcgtccttgcgcagcgaggacacggccgtctattactgcaacttctatgatgtctactcggaaggtgcgttggactattggggacagggaacccttgtgaccgtctctagt

SEQ ID NO: 112 - protein, 120 aa, Homo sapiens
QVQLVQSGAEVKKPGASVKVSCKASGFNIKDYYMHWVRQAPGQGLEWMGWIDPENGDTEYAPKFQGRVTMTADTSTSTVYMELSSLRSEDTAVYYCNFYDVYSEGALDYWGQGTLVTVSS

SEQ ID NO: 113 - DNA, 321 bp, Homo sapiens
gacattcagatgactcaatcaccctcgtccctctcagcttccgtcggtgatagggtaacaatcacatgtcaagcgagcgagaacatctatttctcgcttgcgtggtatcagcagaagcctgggaaagcgcccaagttgctgatctacaatgccaacaatttggaggatggggtgccatcgagattttcgggatccggcagcggaactgacttcacgttcaccattagctcgcttcagccggaggacattgccacctactattgcaaagaagcatacgattcaccgtttacgtttggacaggggacaaagctcgaaatcaaa

SEQ ID NO: 114 - protein, 107 aa, Homo sapiens
DIQMTQSPSSLSASVGDRVTITCQASENIYFSLAWYQQKPGKAPKLLIYNANNLEDGVPSRFSGSGSGTDFTFTISSLQPEDIATYYCKEAYDSPFTFGQGTKLEIK

SEQ ID NO: 115 - DNA, 357 bp, Homo sapiens
caagtacaattggtgcagtcaggagcagaagtcaagaagccgggtgctagcgtgaaagtcagctgtaagacttcgggatatactttcacgagctactggatgcactgggtccgccaggccccaggccaggggcttgagtggatgggtgcgctgtaccccggaaattcggtcacaaactataaccagaagttcaaagggagggtgacaatgaccgcggacacgtcaacgtccactgtatacatggagctgtcctcgctcagatcagaggatacggcggtgtactattgcacacgggggtttttgacagccccttactttgactcgtggggacaggggaccaccgtgaccgtctctagt

SEQ ID NO: 116 - protein, 119 aa, Homo sapiens
QVQLVQSGAEVKKPGASVKVSCKTSGYTFTSYWMHWVRQAPGQGLEWMGALYPGNSVTNYNQKFKGRVTMTADTSTSTVYMELSSLRSEDTAVYYCTRGFLTAPYFDSWGQGTTVTVSS

SEQ ID NO: 117 - DNA, 321 bp, Homo sapiens
gacattcagatgactcaatcaccctcgtccctctcagcttccgtcggtgatagggtaacaatcacatgtcaagcgagcgagaacatctatttctcgcttgcgtggtatcagcagaagcctgggaaagcgcccaagttgctgatctacaatgccaacaatttggaggatggggtgccatcgagattttcgggatccggcagcggaactgactacacgttcaccattagctcgcttcagccggaggacattgccacctacttctgcaaagaagcatacgattcaccgtttacgtttggacaggggacaaagctcgaaatcaaa

SEQ ID NO: 118 - protein, 107 aa, Homo sapiens
DIQMTQSPSSLSASVGDRVTITCQASENIYFSLAWYQQKPGKAPKLLIYNANNLEDGVPSRFSGSGSGTDYTFTISSLQPEDIATYFCKEAYDSPFTFGQGTKLEIK

SEQ ID NO: 119 - DNA, 357 bp, Homo sapiens
caaatccaattggtgcagtcaggagcagaagtcaagaagccgggtgctagcgtgaaagtcagctgtaagacttcgggatatactttcacgagctactggatgcactgggtccgccaggccccaggccaggggcttgagtggatgggtgcgctgtaccccggaaattcggtcacaaactataaccagaagttcaaagggagggccaagctgaccgcggacacgtcaacgtccactgcctacatggagctgtcctcgctcagatcagaggatacggcggtgtactattgcacacgggggtttttgacagccccttactttgactcgtggggacaggggaccaccgtgaccgtctctagt

SEQ ID NO: 120 - protein, 119 aa, Homo sapiens
QIQLVQSGAEVKKPGASVKVSCKTSGYTFTSYWMHWVRQAPGQGLEWMGALYPGNSVTNYNQKFKGRAKLTADTSTSTAYMELSSLRSEDTAVYYCTRGFLTAPYFDSWGQGTTVTVSS

SEQ ID NO: 121 - DNA, 336 bp, Homo sapiens
gatatcgtaatgacgcaatccccggactcacttgccgtgtcgcttggcgaaagagctaccattaactgcaagtcatcccagtcattgttgcattcgtcgaaccagaggaattacctggcgtggtaccaacaaaagcctggacagccacccaaattgttgatctattgggcgtcaattcgcgaaagcggggtccccgaccggttctcgggaagcggttccggtactgactttacactcacgatcagctcgctccaggcagaggatgtggcggtatactattgtcaccagtattactcatactcgacattcgggcagggaaccaaactggagatcaaa

SEQ ID NO: 122 - protein, 112 aa, Homo sapiens
DIVMTQSPDSLAVSLGERATINCKSSQSLLHSSNQRNYLAWYQQKPGQPPKLLIYWASIRESGVPDRFSGSGSGTDFTLTISSLQAEDVAVYYCHQYYSYSTFGQGTKLEIK

SEQ ID NO: 123 - DNA, 369 bp, Homo sapiens
caggtcacacttaaggagtcgggtccagcgctcgtgaagcccacacagaccttgaccctcacgtgtacgttctcgggattttcacttacgactagcgggatgggcgtaagctggattcggcaacctccggggaaagcgctggaatggttggcacacatcttctgggatgatgacaaaaggtataacccctcgctcacgtcgcgcctgacaatctcaaaggacacctccaaaaaccaggtagtgcttacgatgacgaatatggatcccgtggacacagcaacttactactgcgccagaggaggagattactattccacagggtttggttttgactactgggggcagggaactctggtcaccgtctctagt

SEQ ID NO: 124 - protein, 123 aa, Homo sapiens
QVTLKESGPALVKPTQTLTLTCTFSGFSLTTSGMGVSWIRQPPGKALEWLAHIFWDDDKRYNPSLTSRLTISKDTSKNQVVLTMTNMDPVDTATYYCARGGDYYSTGFGFDYWGQGTLVTVSS

SEQ ID NO: 125 - DNA, 336 bp, Homo sapiens
gatatcgtaatgacgcaatccccggactcacttgccgtgtcgcttggcgaaagagctaccattaactgcaagtcatcccagtcattgttgcattcgtcgaaccagaggaattacctggcgtggtaccaacaaaagcctggacagccacccaaattgttgatcagctgggcgtcaattcgcgaaagcggggtccccgaccggttctcgggaagcggttccggtactgactttacactcacgatcagctcgctccaggcagaggatgtggcggtatactattgtcaccagtattactcatactcgacattcgggcagggaaccaaactggagatcaaa

SEQ ID NO: 126 - protein, 112 aa, Homo sapiens
DIVMTQSPDSLAVSLGERATINCKSSQSLLHSSNQRNYLAWYQQKPGQPPKLLISWASIRESGVPDRFSGSGSGTDFTLTISSLQAEDVAVYYCHQYYSYSTFGQGTKLEIK

SEQ ID NO: 127 - DNA, 369 bp, Homo sapiens
caggtcacacttaaggagtcgggtccagcgctcgtgaagcccacacagaccttgaccctcacgtgtacgttctcgggattttcacttagcactagcgggatgggcgtaagctggattcggcaacctccggggaaagcgctggaatggttggcacacatcttctgggatgatgacaaaaggtataacccctcgctcacgtcgcgcctgacaatctcaaaggacacctccaaaaaccaggtagtgcttacgatgacgaatatggatcccgtggacacagcaacttactactgcgccagaggaggagattactattccacagggtttggttttgactactgggggcagggaactctggtcaccgtctctagt

SEQ ID NO: 128 - protein, 123 aa, Homo sapiens
QVTLKESGPALVKPTQTLTLTCTFSGFSLSTSGMGVSWIRQPPGKALEWLAHIFWDDDKRYNPSLTSRLTISKDTSKNQVVLTMTNMDPVDTATYYCARGGDYYSTGFGFDYWGQGTLVTVSS

SEQ ID NO: 129 - DNA, 642 bp, Homo sapiens
cagtacgaattgactcagccaccctcagtggccgtgtcccctggaaagacagccagcatcacctgcgctggagatgaattgggtaataaatatgctgcgtggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagaagtagttatcatgtggtattcggcggagggaccaagctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 130 - protein, 214 aa, Homo sapiens
QYELTQPPSVAVSPGKTASITCAGDELGNKYAAWYQQKPGQAPVLVVYDDSDRPSGIPERFSGSNSGNTATLTISRVEAGDEADYYCQVWDRSSYHVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 131 - protein, 1332 aa, Homo sapiens
GAAGTTCAATTGTTAGAGTCTGGTGGCGGTCTTGTTCAGCCTGGTGGTTCTTTACGTCTTTCTTGCGCTGCTTCCGGATTCACTTTCTCTCTTTACACTATGCAGTGGGTTCGCCAAGCTCCTGGTAAAGGTTTGGAGTGGGTTTCTGGTATCGGTTCTTCTGGTGGCGGTACTTCTTATGCTGACTCCGTTAAAGGTCGCTTCACTATCTCTAGAGACAACTCTAAGAATACTCTCTACTTGCAGATGAACAGCTTAAGGGCTGAGGACACTGCCGTGTATTACTGTGCGAGAGGGGTCAGCAGTTGGTTTTTCGAGTACTGGGGCCAGGGAACCCTGGTCACCGTCTCTAGTGCCTCCACCAAGGGCCCATCGGTCTTCCCCCTGGCGCCCTGCTCCAGGAGCACCTCCGAGAGCACAGCGGCCCTGGGCTGCCTGGTCAAGGACTACTTCCCCGAACCGGTGACGGTGTCGTGGAACTCAGGCGCTCTGACCAGCGGCGTGCACACCTTCCCAGCTGTCCTACAGTCCTCAGGACTCTACTCCCTCAGCAGCGTGGTGACCGTGCCCTCCAGCAACTTCGGCACCCAGACCTACACCTGCAACGTAGATCACAAGCCCAGCAACACCAAGGTGGACAAGACAGTTGAGCGCAAATGTTGTGTCGAGTGCCCACCGTGCCCAGCACCACCTGTGGCAGGACCGTCAGTCTTCCTCTTCCCCCCAAAACCCAAGGACACCCTCATGATCTCCCGGACCCCTGAGGTCACGTGCGTGGTGGTGGACGTGAGCCACGAAGACCCCGAGGTCCAGTTCAACTGGTACGTGGACGGCGTGGAGGTGCATAATGCCAAGACAAAGCCACGGGAGGAGCAGTTCAACAGCACGTTCCGTGTGGTCAGCGTCCTCACCGTTGTGCACCAGGACTGGCTGAACGGCAAGGAGTACAAGTGCAAGGTCTCCAACAAAGGCCTCCCAGCCCCCATCGAGAAAACCATCTCCAAAACCAAAGGGCAGCCCCGAGAACCACAGGTGTACACCCTGCCCCCATCCCGGGAGGAGATGACCAAGAACCAGGTCAGCCTGACCTGCCTGGTCAAAGGCTTCTACCCCAGCGACATCGCCGTGGAGTGGGAGAGCAATGGGCAGCCGGAGAACAACTACAAGACCACACCTCCCATGCTGGACTCCGACGGCTCCTTCTTCCTCTACAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCGGGTAAA

SEQ ID NO: 132 - protein, 444 aa, Homo sapiens
EVQLLESGGGLVQPGGSLRLSCAASGFTFSLYTMQWVRQAPGKGLEWVSGIGSSGGGTSYADSVKGRFTISRDNSKNTLYLQMNSLRAEDTAVYYCARGVSSWFFEYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 133 - protein, 11 aa, Homo sapiens
AGDELGNKYAA

SEQ ID NO: 134 - protein, 7 aa, Homo sapiens
DDSDRPS

SEQ ID NO: 135 - protein, 11 aa, Homo sapiens
QVWDRSSYHVV

SEQ ID NO: 136 - protein, 5 aa, Homo sapiens
LYTMQ

SEQ ID NO: 137 - protein, 17 aa, Homo sapiens
GIGSSGGGTSYADSVKG

SEQ ID NO: 138 - protein, 9 aa, Homo sapiens
GVSSWFFEY

(19) United States
(12) Patent Application Publication
(10) Pub. No.: US 2012/0288507 A1
(43) Pub. Date: Nov. 15, 2012

(54) WISE BINDING AGENTS AND EPITOPES

(75) Inventors: Xueming Qian, Oak Park, CA (US); Aaron George Winters, Ventura, CA (US); Michelle Hortter, Camarillo, CA (US); Kevin Graham, Thousand Oaks, CA (US); Mei-Mei Tsai, Thousand Oaks, CA (US)

(73) Assignee: Amgen Inc., Thousand Oaks, CA (US)

(21) Appl. No.: 13/516,161

(22) PCT Filed: Dec. 17, 2010
(86) PCT No.: PCT/US10/60992

§ 371 Date: Jun. 14, 2012

Related U.S. Application Data
(60) Provisional application No. 61/288,171, filed on Dec. 18, 2009.

(51) Int. Cl. C07K 16/18; A61P 13/12; C07K 19/00; A61K 39/395; C07K 14/47

(52) U.S. Cl. 424/139.1; 530/389.1; 530/387.9; 530/350; 424/172.1; 530/362; 530/391.1

(57) ABSTRACT

The present invention relates to binding agents for WISE, and includes methods for their manufacture and use.

This application claims the benefit of U.S. Provisional Application No. 61/288,171, filed Dec. 18, 2009, which is hereby incorporated by reference.

The present application is being filed along with a Sequence Listing in electronic format. The Sequence Listing is provided as a file entitled A-1532-WO-PCT_SeqList.txt, created Dec. 1, 2010, which is 86.5 KB in size. The information in the electronic format of the Sequence Listing is incorporated herein by reference in its entirety.

### TECHNICAL FIELD

The present invention relates generally to epitopes of WISE protein, including human WISE protein, and binding agents, such as antibodies, capable of binding to WISE or the epitopes herein.

### BACKGROUND OF THE INVENTION

Fibrosis is generally defined as the development of extra connective tissue as part of the healing process and includes a diverse set of symptoms. Excessive fibrosis is a grievous problem that has few therapeutic options.

Cystine knot-containing proteins are typically important regulators of key functions and affect diverse cell types. Wise (USAG-1, SOSTDC1) is a secreted, cystine knot-containing protein and is expressed primarily in the kidney, lungs, skin and epithelial cells. WISE KO mice are fertile and their kidneys have normal function. However when challenged to develop kidney injury either by unilateral uretheral obstruction (UUO) or injection of chemotoxic agent Cisplatin, the WISE KO mice are protected (Yanagita et al., J. Clin Invest. 2006 Jan. 4; 116(1): 70-79). In the UUO model, there is much less fibrosis in the affected kidney in WISE KO mice and expressed much less aSMA, a marker of myofibroblast activation, and preserved the expression of epithelial cell marker E-cadherin. In a Cisplatin model for kidney injury, WISE deletion protected the animal from tubular injury and reduced mortality (Tanaka et al., Kidney International advance online publication 17 Oct. 2007). In addition, when WISE KO mice (aka USAG-1 KO mice) were breed with Col4a3 KO mice, the double knockout mice had significantly less proteinuria and developed less end stage renal disease relative to the Col4a3 KO mice with WT WISE gene. At 4 weeks of age, USAG-1+/+, 3(IV)−/− mice already showed severe proteinuria with extensive splitting of glomerular basement membrane (GBM), while double KO mice showed normal structure of GBM. At 10 weeks of age, USAG-1+/+, 3(IV)−/− mice developed end-stage renal disease, while double KO mice showed significantly preserved renal function with less renal histological changes. (Tanaka et al. J Clin Invest. 2010; 120(3):768-777 and Abstract TH-FC059 2008 ASN meeting).

These data suggest that WISE could be a regulator of adult kidney function. However these studies were limited to knock out mice lacking WISE for their entire development cycle, accordingly it was unpredictable whether acute inhibition of WISE activity using an inhibitor such as an antibody could provide therapeutic benefit to preserve kidney function under pathological conditions associated with various fibrotic diseases.

The present inventors demonstrate it is possible to treat lung and kidney disorders associated with damage and repair including fibrosis and organ dysfunction using binding agents that target WISE.

### BRIEF SUMMARY OF THE INVENTION

Disclosed herein are compositions and methods that can be used to prevent or treat diseases and disorders. The invention further relates to regions of human WISE recognized by the binding agents disclosed herein, methods of using these regions, and methods of making such regions. The invention also relates to epitopes specific to the region of WISE identified as the cystine knot domain, and binding agents which specifically bind to that region.

The invention relates to binding agents, such as antibodies, that specifically bind to WISE. The binding agents can be characterized by their ability to cross-block the binding of at least one antibody disclosed herein to WISE and/or to be cross-blocked from binding WISE by at least one of said antibodies. The antibodies and other binding agents of the invention can also be characterized by their binding pattern to human WISE peptides in a human WISE peptide epitope competition binding assay as disclosed herein.

In certain embodiments, the invention relates to binding agents such as antibodies that inhibit WISE activity and that can decrease tissue injury and associated fibrosis in tissues such as the kidneys, lungs, skin, eye, liver and heart. In addition, the invention relates to binding agents that inhibit proteinuria or proteinuria induced injuries, e.g. fibrosis, which is associated with various immunological and non-immune mediated renal diseases such as in patients with diabetic nephropathy, glomerulonephritis, membranous nephropathy, lupus, transplantation and other renal diseases involving manifestation of increased proteinuria. Furthermore the invention relates to binding agents that improve the function of organs or delay the loss of function in organs mentioned above that are impacted due to either fibrosis and/or proteinuria including but not limited to diseases such as chronic kidney diseases, chronic allograft nephropathy, idiopathic pulmonary fibrosis, cardiomyopathy, glaucoma (lens cell fibrosis) and scleroderma (skin fibrosis). In addition, as tumor metastasis also using similar mechanisms to those used in tissue fibrosis, WISE binding agent may also have utility in delaying tumor metastasis and/or cancer progression.

In other embodiments, the invention relates to binding agents, such as antibodies, that can block the inhibitory effect of WISE in a cell based assay. The invention also relates to binding agents, such as antibodies, that can alter the effect of WISE in a cell based assay. The invention also relates to binding agents, such as antibodies, that can activate the effect of WISE in a cell based assay.

The invention further relates in part to polypeptide constructs comprising two, three, or four polypeptide fragments linked by at least one disulfide bond, representing a core region of the cystine-knot of WISE, and antibodies capable of specifically binding thereto.

In one embodiment, the invention relates to methods of obtaining epitopes suitable for use as immunogens for generating, in mammals, binding agents, such as antibodies capable of binding specifically to WISE; in certain embodiments the binding agents generated are capable of neutralizing WISE activity in vitro and/or in vivo.

In another embodiment, the invention relates to a composition for eliciting an antibody specific for WISE when the composition is administered to an animal, the composition comprising a polypeptide having a sequence selected from but not limited to one of the sequences in the following table:

TABLE 1

| | |
|---|---|
| Seq Id No.: 1 | ATGCTTCCTCCTG CCATTCATTT CTATCTCCTT CCCCTTGCAT |
| | GCATCCTAAT GAAAAGCTGT TTGGCTTTTA AAAATGATGC |
| | CACAGAAATC CTTTATTCAC ATGTGGTTAA ACCTGTTCCA |
| | GCACACCCCA GCAGCAACAG CACGTTGAAT CAAGCCAGAA |
| | ATGGAGGCAG GCATITCAGT AACACTGGAC TGGATCGGAA |
| | CACTCGGGTT CAAGTGGGTT GCCGGGAACT GCGTTCCACC |
| | AAATACATCT CTGATGGCCA GTGCACCAGC ATCAGCCCTC |
| | TGAAGGAGCT GGTGTGTGCT GGCGAGTGCT TGCCCCTGCC |
| | AGTGCTCCCT AACTGGATTG GAGGAGGCTA TGGAACAAAG |
| | TACTGGAGCA GGAGGAGCTC CCAGGAGTGG CGGTGTGTCA |
| | ATGACAAAAC CCGTACCCAG AGAATCCAGC TGCAGTGCCA |
| | AGATGGCAGC ACACGCACCT ACAAAATCAC AGTAGTCACT |
| | GCCTGCAAGT GCAAGAGGTA CACCCGGCAG CACAACGAGT |
| | CCAGTCACAA CTTTGAGAGC ATGTCACCTG CCAAGCCAGT |
| | CCAGCATCAC AGAGAGCGGA AAAGAGCCAG CAAATCCAGC |
| | AAGCACAGCA TGAGTTAGCT CGAGGGGCGG ATCCCCCGGG |
| | CTGCAGGAAT TCGATATCAA GCTTGCTAGC |
| Seq Id No.: 2 | MLPPAIHFYL LPLACILMKS CLAFKNDATE ILYSHVVKPV |
| | PAHPSSNSTL NQARNGGRHF SNTGLDRNTR VQVGCRELRS |
| | TKYISDGQCT SISPLKELVC AGECLPLPVL PNWIGGGYGT |
| | KYWSRRSSQE WRCVNDKTRT QRIQLQCQDG STRTYKITVV |
| | TACKCKRYTR QHNESSHNFE SMSPAKPVQH HRERKRASKS |
| | SKHSMS (human) |
| Seq Id No.: 3 | ATGC TTCCTCCTGC CATTCATCTC |
| | TCTCTCATTCCCCTGCTCTG CATCCTGATG AGAAACTGTT |
| | TGGCTTTTAA AAATGATGCC ACAGAAATCCTTTATTCACA |
| | TGTGGTTAAA CCTGTCCCGG CACACCCCAG CAGCAACAGC |
| | ACCCTGAATCAAGCCAGGAA TGGAGGCAGG CATTTCAGTA |
| | GCACTGGACT GGATCGAAAC AGTCGAGTTCAAGTGGGCTG |
| | CAGGGAACTG CGGTCCACCA AATACATTTC GGACGGCCAG |
| | TGCACCAGCATCAGCCCTCT GAAGGAGCTG GTGTGCGCGG |
| | GCGAGTGCTT GCCCCTGCCG GTGCTTCCCAACTGGATCGG |
| | AGGAGGCTAT GGAACAAAGT ACTGGAGCCG GAGGAGCTCT |
| | CAGGAGTGGCGGTGTGTCAA CGACAAGACG CGCACCCAGA |
| | GGATCCAGCT GCAGTGTCAG GACGGCAGCACGCGCACCTA |
| | CAAAATCACC GTGGTCACGG CGTGCAAGTG CAAGAGGTAC |
| | ACCCGTCAGCACAACGAGTC CAGCCACAAC TTTGAAAGCG |
| | TGTCGCCCGC CAAGCCCGCC CAGCACCACAGAGAGCGGAA |
| | GAGAGCCAGC AAATCCAGCA AGCACAGTCT GAGCTAGCTC |
| | GAG |
| Seq Id No.: 4 | MLPPAIHLSL IPLLCILMRN CLAFKNDATE ILYSHVVKPV |
| | PAHPSSNSTL NQARNGGRHF SSTGLDRNSR VQVGCRELRS |
| | TKYISDGQCT SISPLKELVC AGECLPLPVL PNWIGGGYGT |
| | KYWSRRSSQE WRCVNDKTRT QRIQLQCQDG STRTYKITVV |
| | TACKCKRYTR QHNESSHNFE SVSPAKPAQH HRERKRASKS |
| | SKHSLS (mouse) |
| Seq Id No.: 5 | ATGCT TCCTCCTGCC ATTCATCTCT CTCTCATTCC |
| | CCTGCTCTGCATCCTGATGA AAAACTGTTT GGCTTTTAAA |
| | AATGATGCCA CAGAAATCCT TTATTCACATGTGGTTAAAC |
| | CTGTTTCAGC ACACCCCAGC AGCAACAGCA CCTTGAATCA |
| | AGCCAGGAATGGAGGCAGGC ACTTCAGTAG CACGGGACTG |
| | GATCGAAATA GTCGAGTTCA AGTGGGCTGCAGGGAACTGC |
| | GGTCCACCAA ATACATCTCG GATGGCCAGT GCACCAGCAT |
| | CAGCCCTCTGAAGGAGCTGG TGTGCGCGGG TGAGTGCTTG |
| | CCCTTGCCAG TGCTTCCCAA CTGGATCGGAGGAGGCTACG |
| | GAACAAAGTA CTGGAGCCGG AGGAGCTCCC AGGAGTGGCG |
| | GTGTGTCAACGACAAGACGC GCACCCAGAG AATCCAGCTG |
| | CAGTGTCAGG ACGGCAGCAC ACGCACCTACAAAATCACCG |
| | TGGTCACAGC GTGCAAGTGC AAGAGGTACA CCCGGCAGCA |
| | CAACGAGTCCAGCCACAACT TTGAAAGCGT GTCTCCCGCC |
| | AAGCCCGCCC AGCACCACAG AGAGCGGAAGAGAGCCAGCA |
| | AATCCAGCAA GCACAGTCTG AGCTAGGCGG CCGC |
| Seq Id No.: 6 | MLPPAIHFYL LPLACILMKS CLAFKNDATE ILYSHVVKPV |
| | PAHPSSNSTM NQARNGGRHF SNTGLDRNTR VQVGCRELRS |
| | TKYISDGQCT SISPLKELVC AGECLPLPVL PNWIGGGYGT |
| | KYWSRRSSQE WRCVNDKTRT QRIQLQCQDG STRTYKITVV |
| | TACKCKRYTR QHNESSHNFE SMSPAKPVQH HRERKRASKS |
| | SKHSMS (rat) |
| Seq Id No.: 7 | ATGCT TCCTCCTGCC ATTCATCTCT CTCTCATTCC |
| | CCTGCTCTGCATCCTGATGA AAAACTGTTT GGCTTTTAAA |
| | AATGATGCCA CAGAAATCCT TTATTCACATGTGGTTAAAC |
| | CTGTTTCAGC ACACCCCAGC AGCAACAGCA CCTTGAATCA |
| | AGCCAGGAATGGAGGCAGGC ACTTCAGTAG CACGGGACTG |
| | GATCGAAATA GTCGAGTTCA AGTGGGCTGCAGGGAACTGC |
| | GGTCCACCAA ATACATCTCG GATGGCCAGT GCACCAGCAT |
| | CAGCCCTCTGAAGGAGCTGG TGTGCGCGGG TGAGTGCTTG |
| | CCCTTGCCAG TGCTTCCCAA CTGGATCGGAGGAGGCTACG |
| | GAACAAAGTA CTGGAGCCGG AGGAGCTCCC AGGAGTGGCG |
| | GTGTGTCAACGACAAGACGC GCACCCAGAG AATCCAGCTG |
| | CAGTGTCAGG ACGGCAGCAC ACGCACCTACAAAATCACCG |
| | TGGTCACAGC GTGCAAGTGC AAGAGGTACA CCCGGCAGCA |
| | CAACGAGTCCAGCCACAACT TTGAAAGCGT GTCTCCCGCC |
| | AAGCCCGCCC AGCACCACAG AGAGCGGAAGAGAGCCAGCA |
| | AATCCAGCAA GCACAGTCTG AGCTAGGCGG CCGC |
| Seq Id No.: 8 | MLPPAIHLSL IPLLCILMKN CLAFKNDATE ILYSHVVKPV |
| | SAHPSSNSTL NQARNGGRHF SSTGLDRNSR VQVGCRELRS |
| | TKYISDGQCT SISPLKELVC AGECLPLPVL PNWIGGGYGT |
| | KYWSRRSSQE WRCVNDKTRT QRIQLQCQDG STRTYKITVV |
| | TACKCKRYTR QHNESSHNFE 181 SVSPAKPAQH HRERKRASKS |
| | SKHSLS* (cynomolgus) |
| Seq Id No.: 9 | LPLPVLPNWIGGGYGTKYWSRRSSQEWR |

In other embodiments, the invention also relates to a composition for eliciting an antibody specific for WISE when the composition is administered to an animal, the composition comprising at least one polypeptide consisting essentially of the amino acid sequence of human, mouse, rat or cynomolgus WISE.

It will be understood by one of skill in the art that the WISE proteins listed in Table 1 are the full length protein sequences for the respective species and further processing occurs to allow secretion. In a particular embodiment, the signal peptide is the first 23 amino acids (Seq Id Nos.: 2, 4, 6, and 8) and the removal of the signal peptide results in a mature polypeptide. In a specific embodiment, the invention also relates to polypeptide consisting essentially of amino acids 82 to 109 of a mature WISE shown in Table 1 above also depicted, for example, in Seq Id No.: 9; this polypeptide known herein as the WISE loop 2 polypeptide may be obtained by recombinant expression of fragments of the protein, tryptic digestion of human WISE, or chemical synthesis, and the protein may be isolated by HPLC fractionation among other methods. The peptide, if synthesized, could be in linear or circularized form. If the peptide is produced through recombinant expression, it could be fused to other carrier proteins such as Fc fragment or human serum albumin or others that will increase

In one embodiment, the invention relates to a method of generating an antibody capable of specifically binding to WISE, comprising: (a) immunizing an animal with a composition comprising a WISE polypeptide; (b) collecting sera from the animal; and (c) isolating from the sera an antibody capable of specifically binding to and inhibiting the biological activity of WISE where the antibody binds specifically to loop 2 of WISE.

In yet another embodiment, the invention further contemplates a method of selecting an antibody that binds to WISE loop 2 using phage display, and/or improving affinity of a known antibody to WISE using phage display.

In additional embodiments, the invention also relates to a method of generating an antibody capable of specifically binding to WISE, the method comprising: (a) immunizing an animal with a composition comprising a cystine knot-containing fragment of WISE, for example, Seq Id No. 9, or a derivative thereof; (b) collecting sera from the animal; and (c) isolating from the sera an antibody capable of specifically binding to and inhibiting the biological activity of WISE.

In further embodiments, the invention further relates to a method of detecting an anti-WISE antibody in a biological sample, comprising the steps of (a) contacting the biological sample with a polypeptide consisting essentially of a polypeptide having amino acids 24 to 206 of SEQ ID NO: 2, a polypeptide having amino acids 24 to 206 of SEQ ID NO: 4, a polypeptide having amino acids 24 to 206 of SEQ ID NO: 6, a polypeptide having amino acids 24 to 206 of SEQ ID NO: 8, and a peptide such as SEQ ID NO: 9 under conditions allowing a complex to form between the antibody and the polypeptide; and (b) detecting the presence or absence of the complex, wherein the presence of the complex indicates that the biological sample contains an anti-WISE antibody.

In other embodiments, the invention comprises a method of detecting an anti-WISE antibody in a biological sample, comprising the steps of (a) contacting the biological sample with a composition comprising a cystine knot-containing fragment of WISE under conditions allowing a complex to form between the antibody and the polypeptide; and (b) detecting the presence or absence of the complex, wherein the presence of the complex indicates that the biological sample contains an anti-WISE antibody.

In certain embodiments, the invention relates to a WISE binding agent, such as an antibody that cross-blocks the binding of at least one of antibodies of the invention. Antibodies of the invention include those that bind to loop 2 of WISE polypeptides. Examples of loop 2 binding antibodies are included in Table 2 where variable regions of antibodies that bind to loop 2 of a WISE protein are depicted. In other embodiments, the invention relates to a WISE binding agent, such as an antibody that cross-blocks the binding of at least one of antibodies in Table 2 to a WISE protein.

TABLE 2

| | |
|---|---|
| SEQUENCE | GACATTGTGATGTCACAGTCTCCATCCTCCCTGGCTGTGTCAG |
| ID NO.: 10 | CAGGAGAGAAGGTCACTATGAGCTGCAAATCCAGTCAGAGTC |
| Ab-AA | TGCTCAACAGTAGAACCCGAAAGAACTACTTGGCTTGGTACCA |
| LIGHT | GCAGAAACCAGGGCAGTCTCCTAAACTGCTGATCTACTGGGCA |
| CHAIN | TCCACTAGGCAATCTGGGGTCCCTGATCGCTTCACAGGCAGTG |
| | GATCTGGGACAGATTTCACTCTCACCATCAGCAGTGTGCAGGC |
| | TGAAGACCTGGCAGTTTATTACTGCAAGCAATCTTATAATCTC |
| | CTCACGTTCGGTGCTGGGACCAAGCTGGAGCTGAAA |
| SEQUENCE | DIVMSQSPSSLAVSAGEKVTMSCKSSQSLLNSRTRKNYLAWYQQ |
| ID NO.: 11 | KPGQSPKLLIYWASTRQSGVPDRFTGSGSGTDFTLTISSVQAEDLA |
| Ab-AA | VYYCKQSYNLLTFGAGTKLELK |
| LIGHT | |
| CHAIN | |
| SEQUENCE | GAGGTTCAGCTGCAGCAGTCTGGGGCAGAGCTTGTGAGGTCA |
| ID NO.: 12 | GGGGCCTCAGTCAAGTTGTCCTGCACAGCTTCTGGCTTCAACA |
| Ab-AA | TTAAAGACTACTATATACACTGGATGAAGCAGAGGCCTGAAC |
| HEAVY | AGGGCCTCGAGTGGATTGGATGGATTGATCCTGAGAATGGTG |
| CHAIN | ATACTGAATCTGCCCCGAAGTTCCAGGGCAAGGCCACTATGAC |
| | TGCAGACACATCCTCCAACACAGCCTACCTGCACCTCAGCAGC |
| | CTGACATTTGAGGACACTGCCGTCTATTACTGTAATGCAGAAG |
| | GTTACGGTAGTAGGCACTGGTACTTCGATGTCTGGGGCGCAGG |
| | GACCACGGTCACCGTCTCCTCA |
| SEQUENCE | EVQLQQSGAELVRSGASVKLSCTASGFNIKDYYIHWMKQRPEQG |
| ID NO.: 13 | LEWIGWIDPENGDTESAPKFQGKATMTADTSSNTAYLHLSSLTFE |
| Ab-AA | DTAVYYCNAEGYGSRHWYFDVWGAGTTVTVSS |
| HEAVY | |
| CHAIN | |
| SEQUENCE | GACATCCAGATGACTCAGTCTCCAGCCTCCCTGGCTGCATCTG |
| ID NO.: 14 | TGGGAGAAACCATCACCATCACATGTCAAGCAAGTGAGAACA |
| Ab-AB | TTTACTTCAGTTTAGCATGGTATCAGCAGAAGCAAGGGAAATC |
| LIGHT | TCCTCAGCTCCTGATCTATAATGCAAACAACTTGGAAGATGGT |
| CHAIN | GTCCCATCGAGGTTCAGTGGCAGTGGATCTGGGACACAGTATT |
| | CTATGAAGATCAACAACATGCAGCCTGAAGATACTGCAACTTA |
| | TTTCTGTAAAGAGGCTTATGACTCTCCATTCACGTTCGGCACG |
| | GGGACAAAATTGGAAATAAAA |
| SEQUENCE | DIQMTQSPASLAASVGETITITCQASENIYFSLAWYQQKQGKSPQL |
| ID NO.: 15 | LIYNANNLEDGVPSRFSGSGSGTQYSMKINNMQPEDTATYFCKE |
| Ab-AB | AYDSPFTFGTGTKLEIK |
| LIGHT | |
| CHAIN | |
| SEQUENCE | GAAATTCAACTCCAGCAGTCTGGGACTGTGCTGACAAGGCCTG |
| ID NO.: 16 | GGGCTTCAGTGAAGATGTCCTGCAAGACTTCTGGCTACACCTT |
| Ab-AB | TACCAGCTACTGGATGCACTGGGTAAAACAGAGGCCTGGACA |
| HEAVY | GGGTCTGGAATGGATTGGCGCTCTTTATCCTGGAAATAGTGTT |
| CHAIN | ACTAACTACAACCAGAAGTTCAAGGGCAAGGCCAAACTGACT |
| | GCAGTCACATCCACCAGCACTGCCTACATGGAGCTCAGCAGCC |
| | TGACAAATGAGGACTCTGCGGTCTATTACTGTACAAGAGGATT |
| | TCTTACTGCGCCCTACTTTGACTCCTGGGGCCAAGGCACCACT |
| | CTCACAGTCTCCTCA |
| SEQUENCE | EIQLQQSGTVLTRPGASVKMSCKTSGYTFTSYWMHWVKQRPGQ |
| ID NO.: 17 | GLEWIGALYPGNSVTNYNQKFKGKAKLTAVTSTSTAYMELSSLT |
| Ab-AB | NEDSAVYYCTRGFLTAPYFDSWGQGTTLTVSS |
| HEAVY | |
| CHAIN | |
| SEQUENCE | GACATTGTGGTGTCACAGGCTCCATCCTCCCTTGCTGTGTCAGT |
| ID NO.: 18 | TGGAGAGAAGATTATTATGAGCTGCAAGTCCAGTCAGAGCCTT |
| Ab-AC | TTACACAGCAGCAATCGAAGGAACTACTTGGCCTGGTACCAAC |
| LIGHT | AGAAACCAGGGCAGTCTCCTAAATTGCTGATTTCCTGGGCATC |
| CHAIN | CATTAGGGAATCTGGGGTCCCTGATCGCTTCACAGGCAGTGGA |
| | TCCGGGACAGATTTCACTCTCACCATCAGCAGCGTGAAGACTG |
| | AAGACCTGGCAATTTATTACTGTCACCAATATTATACTTATTCC |
| | ACGTTCGGTGCTGGGACCAAGCTGGAGCTGAAG |
| SEQUENCE | DIVVSQAPSSLAVSVGEKIIMSCKSSQSLLHSSNRRNYLAWYQQK |
| ID NO.: 19 | PGQSPKLLISWASIRESGVPDRFTGSGSGTDFTLTISSVKTEDLAIY |
| Ab-AC | YCHQYYTYSTFGAGTKLELK |
| LIGHT | |
| CHAIN | |
| SEQUENCE | CAGGTTACTCTAAAAGAGTCTGGCCCTGAGATACTGCAGCCCT |
| ID NO.: 20 | CCCAGACCCTCAGTCTGACTTGTTCGTTCTCTGGGTTTTCACTG |
| Ab-AC | ACCACTTCTGGTATGGGTGTGAGCTGGATTCGTCAGCCTTCAG |
| HEAVY | GAGGGAGTCTGGAATGGCTGGCTCACATTTTCTGGGATGATGA |
| CHAIN | CAAGCGGTATAATCCATCCCTGACGAGTCGACTCACAATCTCC |
| | AAGGATGCCCCCAGAAACCAGGTTTTCCTCAAAATCACCAGTG |
| | TGGACACTGCAGATGCTGCCACATATTACTGTGCTCGAGGAGG |
| | AGATTATTACAGTACTGGATTTGGCTTTGATTACTGGGGCCAA |
| | GGGACTCTGGTCACTGTCTCTGCA |
| SEQUENCE | QVTLKESGPEILQPSQTLSLTCSFSGFSLTTSGMGVSWIRQPSGGSL |
| ID NO.: 21 | EWLAHIFWDDDKRYNPSLTSRLTISKDAPRNQVFLKITSVDTADA |
| Ab-AC | ATYYCARGGDYYSTGFGFDYWGQGTLVTVSA |
| HEAVY | |
| CHAIN | |
| SEQUENCE | GACATCCAGATGACTCAGTCTCCAGCCTCCCTGGCTGCATCTG |
| ID NO.: 22 | TGGGAGAATCCATCACCATCACATGTCAGGCAAGTGAGAACA |
| Ab-AD | TTTACTTCAGTTTAGCATGGTATCAGCAGAAGCAAGGGAGGTC |
| LIGHT | TCCTCAGCTCCTGATCTATCATGCAAAAAGTTTGGAAGATGGT |
| CHAIN | GTCCCATCGAGGTTCAGTGGCAGTGGCTCTGGGACACAGTATT |
| | CTATGAAGATCAACAGCATGCAGCCTGAAGATACTGCAACTTA |
| | TTTCTGTAAACAGGCTTATGACCATCCATTCACGTTCGGCACG |
| | GGGACAAAATTGGAAATGAAA |
| SEQUENCE | DIQMTQSPASLAASVGESITITCQASENIYFSLAWYQQKQGRSPQL |
| ID NO.: 23 | LIYHAKSLEDGVPSRFSGSGSGTQYSMKINSMQPEDTATYFCKQA |
| Ab-AD | YDHPFTFGTGTKLEMK |
| LIGHT | |
| CHAIN | |
| SEQUENCE | GAGGTTCAGCTCCAGCAGTCTGGGACTGTGCTGGCAAGGCCTG |
| ID NO.: 24 | GGGCTTCAGTGAAGATGTCCTGTAAGGCTTCTGGCTACACCTT |
| Ab-AD | TACCAGCTACTGGATGCACTGGGTAAAACAGAGGCCTGGACA |
| HEAVY | GGGTCTGGAATGGATTGTCGCTATTTATCCTGGAAATAGTGAT |
| CHAIN | ACTAACTACAACCAGAAGITCAAGGGCAAGGCCAAACTGACT |
| | GCAGTCACATCCACCAGCACTGCCTACATGGAACTCAACAGCC |
| | TGACAAATGAGGACTCTGCGGTCTATTACTGTGTAAGAGGATT |
| | TATTACTGCGCCCTACTTTGACTACTGGGGCCAAGGCACCACT |
| | CTCACAGTCTCCTCA |
| SEQUENCE | EVQLQQSGTVLARPGASVKMSCKASGYTFTSYWMHWVKQRPG |
| ID NO.: 25 | QGLEWIVAIYPGNSDTNYNQKFKGKAKLTAVTSTSTAYMELNSL |
| Ab-AD | TNEDSAVYYCVRGFITAPYFDYWGQGTTLTVSS |
| HEAVY | |
| CHAIN | |
| SEQUENCE | GACATTGTGATGTCACAGTCTCCATCCTCCCTGGCTGTGTCAG |
| ID NO.: 26 | CAGGAGAGAAGGTCACTATGAGCTGCAAATCCAGTCAGAGTC |
| Ab-AE | TGCTCAACAGTAGAACCCGAAAGAACTACTTGGCTTGGTACCA |
| LIGHT | GCAGAAGCCAGGGCAGTCTCCTAAACTGCTGATCTACTGGGCA |
| CHAIN | TCCACTAGGGAATCTGGGGTCCCTGATCGCTTCACAGGCAGTG |
| | GATCTGGGACAGATTTCACTCTCACCATCAGCAGTGTGCAGGC |
| | TGAAGACCTGGCAGTTTATTACTGCAAGCAATCTTATAATCTT |
| | CCGACGTTCGGTGGAGGCACCAGGCTGGAAATCAAA |
| SEQUENCE | DIVMSQSPSSLAVSAGEKVTMSCKSSQSLLNSRTRKNYLAWYQQ |
| ID NO.: 27 | KPGQSPKLLIYWASTRESGVPDRFTGSGSGTDFTLTISSVQAEDLA |
| Ab-AE | VYYCKQSYNLPTFGGGTRLEIK |
| LIGHT | |
| CHAIN | |
| SEQUENCE | GAGGTTCAGCTGCAGCAGTCTGGGGCAGAGCTTGTGAGGTCA |
| ID NO.: 28 | GGGGCCTCAGTCAAGTTGTCCTGCACAGCTTCTGGCTTCAACA |
| Ab-AE | TTAAAGACTACTATATGCACTGGGTGAAGCAGAGGCCTGAAC |
| HEAVY | AGGGCCTGGAGTGGATTGGATGGATTGATCCTGAAAATGGTG |
| CHAIN | ATACTGAATATGCCCCGAAGTTCCAGGGCAAGGCCACTATGAC |
| | TGCAGACACATCCTCCAACACAGCCTACCTGCAGCTCAGCAGC |
| | CTGACATCTGAGGACACTGCCGTCTTTTACTGTAATTTCTATGA |
| | TGTTTACTCCGAGGGGACTATGGCCTACTGGGGTCAAGGAACC |
| | TCAGTCACCGTCTCCTCA |
| SEQUENCE | EVQLQQSGAELVRSGASVKLSCTASGFNIKDYYMHWVKQRPEQ |
| ID NO.: 29 | GLEWIGWIDPENGDTEYAPKFQGKATMTADTSSNTAYLQLSSLT |
| Ab-AE | SEDTAVFYCNFYDVYSEGTMAYWGQGTSVTVSS |
| HEAVY | |
| CHAIN | |
| SEQUENCE | GACATCCAGATGACTCAGTCTCCAGCTTCACTGTCTGCATCTG |
| ID NO.: 30 | TGGGAGAAACTGTCACCATCACATGTGGAGCAAGTGAGAATA |
| Ab-AF | TTTACGGTGCTTTAAATTGGTATCAGCGGAAACAGGGAAAATC |
| LIGHT | TCCTCAGGTCCTGATCTATGGTGCAACCAACTTGGCAGATGGC |
| CHAIN | ATGTCATCGAGGTTCAGTGGCAGTGGATCTGGTAGACAGTATT |
| | CTCTCAAGATCAGTAGCCTGCATCCTGACGATGTTGCAATGTA |
| | TTACTGTCAAAATGTGTTCAGTAGTCCGCTCACGTTCGGTGCT |
| | GGGACCAAGCTGGAGCTGAAA |
| SEQUENCE | DIQMTQSPASLSASVGETVTITCGASENIYGALNWYQRKQGKSPQ |
| ID NO.: 31 | VLIYGATNLADGMSSRFSGSGSGRQYSLKISSLHPDDVAMYYCQ |
| Ab-AF | NVFSSPLTFGAGTKLELK |
| LIGHT | |
| CHAIN | |
| SEQUENCE | CAGATCCAGTTGGTGCAGTCTGGACCTGAGCTGAAGAAGCCTG |
| ID NO.: 32 | GAGAGACAGTCAAGATCTCCTGCAAGGCTTCTGGTTATACCTT |
| Ab-AF | CACAGACTATTCAATGCACTGGGTGAAGCAGGCTCCAGGAAA |
| HEAVY | GGGTTTAAAGTGGATGGGCTGGATAAACACTGAGACTGGTGA |
| CHAIN | GCCAACATATGCAGATGACTTCAAGGGACGGTTTGCCTTCTCT |
| | TTGGAAACCTCTGCCAGCACTGCCTGTTTGGAGATCAACAACC |
| | TCAAAAATGAGGACACGGCTACATATTTCTGTTCTTTAACTGG |
| | GTACTGGGGTCAAGGAACCTCAGTCACCGTCTCCTCA |
| SEQUENCE | QIQLVQSGPELKKPGETVKISCKASGYTFTDYSMHWVKQAPGKG |
| ID NO.: 33 | LKWMGWINTETGEPTYADDFKGRFAFSLETSASTACLEINNLKNE |
| Ab-AF | DTATYFCSLTGYWGQGTSVTVSS |
| HEAVY | |
| CHAIN | |
| SEQUENCE | GACATCCAGATGACTCAGTCTCCAGCTTCACTGTCTGCATCTG |
| ID NO.: 70 | TGGGAGAAACTGTCACCATCACATGTGGAGCCAGTGAGAATA |
| Ab-AG | TTTACGGTGCTTTAAATTGGTATCAGCGGAAACAGGGAAAATC |
| Light Chain | TCCTCAGCTCCTGATCTTTGGTGCAACCAACTTGGCAGATGGC |
| | ATGTCATCGAGGTTCAGTGGCAGTGGATCTGGTAGACAGTATT |
| | CTCTCGAGATCAGTAGCCTGCATCCTGACGATGTTGCAACGTA |
| | TTACTGTCAAAATTTATTTAATTCTCCGCTCACATTCGGTGCTG |
| | GGACCAAGCTGGACCTGAAA |
| SEQUENCE | DIQMTQSPASLSASVGETVTITCGASENIYGALNWYQRKQGKSPQ |
| ID NO.: 71 | LLIFGATNLADGMSSRFSGSGSGRQYSLEISSLHPDDVATYYCQN |
| Ab-AG | LFNSPLTFGAGTKLDLK |
| Light Chain | |
| SEQUENCE | CAGATCCAGTTGGTGCAGTCTGGACCTGAGCTGAAGAAGCCTG |
| ID NO.: 72 | GAGAGACAGTCAAGATCTCCTGCAAGGCTTCTGGTTATACCTT |
| Ab-AG | CACAGACTATTCAATGCACTGGGTGAAGCAGGCTCCAGGAAA |
| Heavy Chain | GGGTTTAAAGTGGATGGGCTGGATAAACACTGAGACTGGTGA |
| | GCCAACATATGCAGCTGACTTCAAGGGACGGTTTGCCTTCTCT |
| | TTGGAAACCTCTGCCAGCACTGCCTATTTGCAGATCAACAACC |
| | TCAAAAATGAGGACACGGCTACATATTTCTGTACTTTAACTGG |
| | GTACTGGGGTCAGGGAACCTCAGTCACCGTCTCCTCA |
| SEQUENCE | QIQLVQSGPELKKPGETVKISCKASGYTFTDYSMHWVKQAPGKG |
| ID NO.: 73 | LKWMGWINTETGEPTYAADFKGRFAFSLETSASTAYLQINNLKN |
| Ab-AG | EDTATYFCTLTGYWGQGTSVTVSS |
| Heavy Chain | |
| SEQUENCE | GACATTGTGATGTCACAGTCTCCATCCTCCCTGGCTGTGTCAG |
| ID NO.: 74 | CAGGAGAGAAGGTCACTATGAGCTGCAAATCCAGTCAGAGTC |
| Ab-AH | TGCTCAACAGTAGAACCCGAAAGAACTACTTGGCTTGGTACCA |
| Light Chain | GCAGAAACCAGGGCAGTCTCCTAAACTGCTGATCTACTGGGCA |
| | TCCACTAGGAAATCTGGGGTCCCTGATCGCTTCATAGGCAGTG |
| | GATCTGGGACAGATTTCACTCTCACCATTAGCAGTGTGCAGGC |
| | TGAAGACCTGGCAGTTTATTACTGCAAGCAATCTTATAATCTC |
| | GTCACGTTCGGTGCTGGGACCAAGCTGGAGCTGAAA |
| SEQUENCE | DIVMSQSPSSLAVSAGEKVTMSCKSSQSLLNSRTRKNYLAWYQQ |
| ID NO.: 75 | KPGQSPKLLIYWASTRKSGVPDRFIGSGSGTDFTLTISSVQAEDLA |
| Ab-AH | VYYCKQSYNLVTFGAGTKLELK |
| Light Chain | |
| SEQUENCE | GAGGTTCAGCTGCAGCAGTCTGGGGCAGAGCTTGTGAGGTCA |
| ID NO.: 76 | GGGGCCTCAGTCAGGTTGTCCTGCACAGCTTCTGGCTTCAACA |
| Ab-AH | TTAAAGACTTCTATATGCACTGGTTGAAGCAGAGGCCTGAACA |
| Heavy Chain | GGGCCTGGAGTGGATTGGATGGATTGATCCTGAGAATGGTGAT |
| | ACTGAGTCTGCCCCGAAGTTCCAGGGCAAGGCCACTATGACTG |
| | CAGACACATCCTCCAACACAGCCTACCTGCAGCTCAGCAGCCT |
| | GACATCTGAGGACACTGCCGTCTATTGCTGTAATGCAGAAGGC |
| | TACGATAATAGCCACTGGTACTTCGATGTCTGGGGCGCAGGGA |
| | CCACGGTCACCGTCTCCTCA |
| SEQUENCE | EVQLQQSGAELVRSGASVRLSCTASGFNIKDFYMHWLKQRPEQG |
| ID NO.: 77 | LEWIGWIDPENGDTESAPKFQGKATMTADTSSNTAYLQLSSLTSE |
| Ab-AH | DTAVYCCNAEGYDNSHWYFDVWGAGTTVTVSS |
| Heavy Chain | |
| SEQUENCE | GACATTGTGGTGTCACAGGCTCCATCCTCCCTGCTGTGTCAGT |
| ID NO.: 78 | TGGAGAGAAGATTATTATGAGCTGCAAGTCCAGTCAGAGCCTT |
| Ab-AI | TTACACAGCAGCAATCAAAGGAACTACTTGGCCTGGTACCAAC |
| Light Chain | AGAAACCAGGGCAGTCTCCTAAACTGCTGATTTCCTGGGCATC |
| | CATTAGGGAATCTGGGGTCCCTGATCGCTTCACAGGCAGTGGA |
| | TCTGGGACAGATTTCACTCTCACCATCAGCAGCGTGAAGACTG |
| | AAGACCTGGCAGTTTATTATTGTCACCAATATTATAGTTATTCC |
| | ACGTTCGGTGCTGGGACCAAGCTGGAGCTGAAG |
| SEQUENCE | DIVVSQAPSSLAVSVGEKIIMSCKSSQSLLHSSNQRNYLAWYQQK |
| ID NO.: 79 | PGQSPKLLISWASIRESGVPDRFTGSGSGTDFTLTISSVKTEDLAVY |
| Ab-AI | YCHQYYSYSTFGAGTKLELK |
| Light Chain | |
| SEQUENCE | CAGGTTACTCTAAAAGAGTCTGGCCCTGGGATATTGCAGCCCT |
| ID NO.: 80 | CCCAGACCCTCAGTCTGACTTGTTCTTTCTCTGGGTTTTCACTG |
| Ab-AI | ACCACTTCTGGTATGGGTGTGAGCTGGATTCGTCAGCCTTCAG |
| Heavy Chain | GAGGGAGTCTGGAATGGCTGGCACACATTTTCTGGGATGATGA |
| | CAAGCGCTATAATCCATCCCTGACGAGCCGACTCACAATCTCC |
| | AAGGATGCCTCCAGAAACCAGGTTTTCCTCAAGATCAGCAGTG |
| | TGGACACTGCAGACGCTGCCACATACTACTGTGCTCGAGGAGG |
| | AGATTACTACAGTACTGGATTTGGCTTTGATTACTGGGGCCAA |
| | GGGACTCTGGTCACTGTCTCTGCA |
| SEQUENCE | QVTLKESGPGILQPSQTLSLTCSFSGFSLTTSGMGVSWIRQPSGGS |
| ID NO.: 81 | LEWLAHIFWDDDKRYNPSLTSRLTISKDASRNQVFLKISSVDTAD |
| Ab-AI | AATYYCARGGDYYSTGFGFDYWGQGTLVTVSA |
| Heavy Chain | |
| SEQUENCE | GACATTGTGATGTCACAGTCTCCATCCTCCCTGGCTGTGTCAG |
| ID NO.: 82 | CAGGAGAGAAGGTCACTATGAGCTGCAAATCCAGTCAGAGTC |
| Ab-AJ | TGCTCAACAGTAGAACCCGAAAGAACTACTTGGCTTGGTACCA |
| Light Chain | GCAGAAACCAGGGCAGTCTCCTAAACTGCTGATCTACTGGGCA |
| | TCCACTAGGGAATCTGGGGTCCCTGATCGCTTCACAGGCAGTG |
| | GATCTGGGACAGATTTCACTCTCACCATCAGCAGTGTGCAGGC |
| | TGAAGACCTGGCAGTITATTATTGCAAGCAATCTTATAATCTT |
| | CCGACGTTCGGTGGAGGCACCAAGCTGGAAATCAAA |
| SEQUENCE | DIVMSQSPSSLAVSAGEKVTMSCKSSQSLLNSRTRKNYLAWYQQ |
| ID NO.: 83 | KPGQSPKLLIYWASTRESGVPDRFTGSGSGTDFTLTISSVQAEDLA |
| Ab-AJ | VYYCKQSYNLPTFGGGTKLEIK |
| Light Chain | |
| SEQUENCE | GAGGTTCAGCTGCAGCAGTCTGGGGCAGAGCTTGTGAGGTCA |
| ID NO.: 84 | GGGGCCTCAGTCAAGTTGTCCTGCACAGCTTCTGGCTTCAACA |
| Ab-AJ | TTAAAGACTACTATATGCATTGGGTGAAGCAGAGGCCTGAAC |
| Heavy Chain | AGGGCCTGGAGTGGATTGGATGGATTGATCCTGAGAATGGTG |
| | ATACTGAATATGCCCCGAAGTTCCAGGGCAAGGCCACTATGAC |
| | TGCAGACACATCCTCCAACACAGCCTACCTGCAGCTCAGCAGC |
| | CTGACATCTGAGGACACTGCCGTCTATTACTGTAATTTCTATG |
| | ATGTTTACTCCGAGGGGGCTTTGGACTACTGGGGTCAAGGAAC |
| | CTCAGTCACCGTCTCCTCA |
| SEQUENCE | EVQLQQSGAELVRSGASVKLSCTASGFNIKDYYMHWVKQRPEQ |
| ID NO.: 85 | GLEWIGWIDPENGDTEYAPKFQGKATMTADTSSNTAYLQLSSLT |
| Ab-AJ | SEDTAVYYCNFYDVYSEGALDYWGQGTSVTVSS |
| Heavy Chain | |

In certain embodiments of the invention, it is contemplated that the pairs of variable domains of the polypeptides depicted in Seq Id Nos.; 11 and 13, Seq Id Nos.: 15 and 17, Seq Id Nos.: 19 and 21, Seq Id Nos.: 23 and 25, Seq Id Nos. 27 and 29, Seq Id Nos. 31 and 33, Seq Id No. 71 and 73, Seq Id No. 75 and 77, Seq Id No. 79 and 81, and Seq Id No. 83 and 85 are binding domains capable of binding loop 2 of human WISE (Seq Id No.: 9). As used herein the variable domains depicted in particular sequence identifiers as follows, comprise heavy and light chains capable of binding to WISE (Seq Id No.: 9), namely, Seq Id Nos.; 11 and 13 refer to Ab-AA, Seq Id Nos.; 15 and 17 refer to Ab-AB, Seq Id Nos.; 19 and 21 refer to Ab-AC, Seq Id Nos.; 23 and 25 refer to Ab-AD, Seq Id Nos.; 27 and 29 refer to Ab-AE, Seq Id Nos.; 31 and 33 refer to Ab-AF, Seq Id Nos.; 71 and 73 refer to Ab-AG, Seq Id Nos.; 75 and 77 refer to Ab-AH, Seq Id Nos.; 79 and 81 refer to Ab-AI and Seq Id Nos.; 83 and 85 refer to Ab-AJ. It is further contemplated that the complementary determining regions of these variable domains can be cloned into human framework regions of antibodies, e.g., IgG2, such that binding activity is retained for Seq Id No. 9.

The CDR's of the antibody variable domains depicted in Table 2 are presented below in Table 3.

TABLE 3

| | |
|---|---|
| Seq Id No. 34 | KSSQSLLNSRTRKNYLA |
| Seq Id No. 35 | WASTRQS |
| Seq Id No. 36 | KQSYNLLT |
| Seq Id No. 37 | DYYIH |
| Seq Id No. 38 | WIDPENGDTESAPKFQG |
| Seq Id No. 39 | EGYGSRHWYFDV |
| Seq Id No. 40 | QASENIYFSLA |
| Seq Id No. 41 | NANNLED |
| Seq Id No. 42 | KEAYDSPFT |
| Seq Id No. 43 | SYWMH |
| Seq Id No. 44 | ALYPGNSVTNYNQKFKG |
| Seq Id No. 45 | GFLTAPYFDS |
| Seq Id No. 46 | KSSQSLLHSSNRRNYLA |
| Seq Id No. 47 | WASIRES |
| Seq Id No. 48 | HQYYTYST |
| Seq Id No. 49 | TSGMGVS |
| Seq Id No. 50 | HIFWDDDKRYNPSLTS |
| Seq Id No. 51 | GGDYYSTGFGFDY |
| Seq Id No. 52 | QASENIYFSLA |
| Seq Id No. 53 | HAKSLED |
| Seq Id No. 54 | KQAYDHPFT |
| Seq Id No. 55 | SYWMH |
| Seq Id No. 56 | AIYPGNSDTNYNQKFKG |
| Seq Id No. 57 | GFITAPYFDY |
| Seq Id No. 58 | KSSQSLLNSRTRKNYLA |
| Seq Id No. 59 | WASTRES |
| Seq Id No. 60 | KQSYNLPT |
| Seq Id No. 61 | DYYMH |
| Seq Id No. 62 | WIDPENGDTEYAPKFQG |
| Seq Id No. 63 | YDVYSEGTMAY |
| Seq Id No. 64 | GASENIYGALN |
| Seq Id No. 65 | GATNLAD |
| Seq Id No. 66 | QNVFSSPLT |
| Seq Id No. 67 | DYSMH |
| Seq Id No. 68 | WINTETGEPTYADDFKG |
| Seq Id No. 69 | TGY |
| Seq Id No. 86 | GASENIYGALN |
| Seq Id No. 87 | GATNLAD |
| Seq Id No. 88 | QNLFNSPLT |
| Seq Id No. 89 | DYSMH |
| Seq Id No. 90 | WINTETGEPTYAADFKG |
| Seq Id No. 91 | KSSQSLLNSRTRKNYLA |
| Seq Id No. 92 | WASTRKS |
| Seq Id No. 93 | KQSYNLVT |
| Seq Id No. 94 | DFYMH |
| Seq Id No. 95 | WIDPENGDTESAPKFQG |
| Seq Id No. 96 | EGYDNSHWYFDV |
| Seq Id No. 97 | KSSQSLLHSSNQRNYLA |
| Seq Id No. 98 | WASIRES |
| Seq Id No. 99 | HQYYSYST |
| Seq Id No. 100 | TSGMGVS |
| Seq Id No. 101 | HIFWDDDKRYNPSLTS |
| Seq Id No. 102 | GGDYYSTGFGFDY |
| Seq Id No. 103 | KSSQSLLNSRTRKNYLA |
| Seq Id No. 104 | WASTRES |
| Seq Id No. 105 | KQSYNLPT |
| Seq Id No. 106 | DYYMH |
| Seq Id No. 107 | WIDPENGDTEYAPKFQG |
| Seq Id No. 108 | YDVYSEGALDY |

In certain embodiments, an antibody of the invention includes an antibody comprising the complementary determining regions shown in Seq Id Nos.: 34, 35 and 36 and Seq Id Nos.: 37, 38 and 39. In certain embodiments, an antibody of the invention includes an antibody comprising the complementary determining regions shown in Seq Id Nos.: 40, 41 and 42 and Seq Id Nos.: 43, 44 and 45. In certain embodiments, an antibody of the invention includes an antibody comprising the complementary determining regions shown in Seq Id Nos.: 46, 47 and 48 and Seq Id Nos.: 49, 50 and 51. In certain embodiments, an antibody of the invention includes an antibody comprising the complementary determining regions shown in Seq Id Nos.: 52, 53 and 54 and Seq Id Nos.: 55, 56 and 57. In certain embodiments, an antibody of the invention includes an antibody comprising the complementary determining regions shown in Seq Id Nos.: 58, 59 and 60 and Seq Id Nos.: 61, 62 and 63. In certain embodiments, an antibody of the invention includes an antibody comprising the complementary determining regions shown in Seq Id Nos.: 64, 65 and 66 and Seq Id Nos.: 67, 68 and 69. In certain embodiments, an antibody of the invention includes an antibody comprising the complementary determining regions shown in Seq Id Nos.: 86, 87 and 88 and Seq Id Nos.: 89, 90 and 69. In certain embodiments, an antibody of the invention includes an antibody comprising the complementary determining regions shown in Seq Id Nos.: 91, 92 and 93 and Seq Id Nos.: 94, 95 and 96. In certain embodiments, an antibody of the invention includes an antibody comprising the complementary determining regions shown in Seq Id Nos.: 97, 98 and 99 and Seq Id Nos.: 100, 101 and 102. In certain embodiments, an antibody of the invention includes an antibody comprising the complementary determining regions shown in Seq Id Nos.: 103, 104 and 105 and Seq Id Nos.: 106, 107 and 108.

A WISE binding agent of the invention may also be cross-blocked from binding to WISE by at least one of antibodies comprising the variable domains depicted in Table 2. A WISE binding agent of the invention may also be cross-blocked from binding to WISE by at least one of antibodies depicted in Table 2 and/or an antibody comprising the complementarity determining regions exemplified by Seq Id Nos.: 34, 35, and 36, and Seq Id Nos.: 37, 38 and 39; Seq Id Nos.: 40, 41 and 42, and Seq Id Nos.: 43, 44 and 45; Seq Id Nos.: 46, 47 and 48 and Seq Id Nos.: 49, 50 and 51; Seq Id Nos.: 52, 53 and 54 and Seq Id Nos.: 55, 56 and 57; Seq Id Nos.: 58, 59 and 60 and Seq Id Nos.: 61, 62 and 63; Seq Id Nos.: 64, 65 and 66, and Seq Id Nos.: 67, 68 and 69; Seq Id Nos.: 86, 87, and 88, and Seq Id Nos.: 89, 90 and 69; Seq Id Nos.: 91, 92 and 93, and Seq Id Nos.: 94, 95 and 96; Seq Id Nos.: 97, 98 and 99, and Seq Id Nos.: 100, 101 and 102; and Seq Id Nos.: 103, 104 and 105, and Seq Id Nos.: 106, 107 and 108.

Antibodies that comprise the complementarity determining regions (CDRs) of the antibodies exemplified herein include humanized antibodies where the nucleic acids encoding CDRs shown in Seq Id Nos.: 34, 35 and 36, and Seq Id Nos.: 37, 38 and 39; Seq Id Nos.: 40, 41 and 42, and Seq Id Nos.: 43, 44 and 45; Seq Id Nos.: 46, 47 and 48 and Seq Id Nos.: 49, 50 and 51; Seq Id Nos.: 52, 53 and 54 and Seq Id Nos.: 55, 56 and 57; Seq Id Nos.: 58, 59 and 60 and Seq Id Nos.: 61, 62 and 63; Seq Id Nos.: 64, 65 and 66, and Seq Id Nos.: 67, 68 and 69 Seq Id Nos.: 86, 87, and 88, and Seq Id Nos.: 89, 90 and 69; Seq Id Nos.: 91, 92 and 93, and Seq Id Nos.: 94, 95 and 96; Seq Id Nos.: 97, 98 and 99, and Seq Id Nos.: 100, 101 and 102; or Seq Id Nos.: 103, 104 and 105, and Seq Id Nos.: 106, 107 and 108, are cloned into a human framework region using conventional molecular biology techniques. These sequences can be cloned with our without additional modifications to the framework regions and/or the with additional changes to the CDR's. These humanized antibodies are expressed using conventional methods described in part herein. Examples of humanized antibodies of the invention includes those depicted as heavy and light chain pairs shown in Seq Id Nos.: 110 and 112, Seq Id Nos.: 114 and 116 (Seq Id Nos.: 118 and 116, Seq Id Nos.: 114 and 120, Seq Id Nos.: 118 and 120, Seq Id Nos.: 122 and 124, Seq Id Nos.: 126 and 124, Seq Id Nos.: 122 and 128, and Seq Id Nos.: 126 and 128 below in Table 4 with corresponding nucleic acid sequences.

TABLE 4

| | |
|---|---|
| Seq Id No. 109 | GATATCGTAATGACCCAGTCGCCTGACTCACTTGCGGTGTCC |
| | CTCGGGGAAAGAGCTACAATCAATTGCAAGTCAAGCCAGTC |
| | CTTGCTCAACAGCAGGACGCGAAAGAACTACTTGGCGTGGT |
| | ACCAGCAAAAGCCGGGACAACCGCCCAAGTTGCTGATCTAT |
| | TGGGCCTCAACGCGCGAGTCGGGGGTCCCAGACCGGTTCTCG |
| | GGTTCGGGATCCGGGACTGACTTCACGCTGACTATTTCGTCG |
| | TTGCAGGCAGAGGATGTCGCGGTGTATTACTGTAAACAGAG |
| | CTATAACCTTCCCACCTTTGGTGGCGGAACAAAAGTGGAAAT |
| | CAAA |
| Seq Id No. 110 | DIVMTQSPDSLAVSLGERATINCKSSQSLLNSRTRKNYLAWYQ |
| | QKPGQPPKLLIYWASTRESGVPDRFSGSGSGTDFTLTISSLQAED |
| | VAVYYCKQSYNLPTFGGGTKVEIK |
| Seq Id No. 111 | CAGGTACAACTCGTGCAGAGCGGAGCCGAAGTCAAAAAGCC |
| | CGGTGCGTCAGTGAAGGTATCGTGTAAGGCATCAGGGTTTAA |
| | CATCAAAGATTACTACATGCACTGGGTGAGGCAAGCTCCGG |
| | GCCAGGGGCTGGAGTGGATGGGGTGGATTGATCCAGAAAAT |
| | GGAGACACTGAGTATGCACCTAAGTTCCAGGGGAGAGTGAC |
| | GATGACAGCGGACACCTCGACGTCCACAGTGTACATGGAGC |
| | TGTCGTCCTTGCGCAGCGAGGACACGGCCGTCTATTACTGCA |
| | ACTTCTATGATGTCTACTCGGAAGGTGCGTTGGACTATTGGG |
| | GACAGGGAACCCTTGTGACCGTCTCTAGT |
| Seq Id No. 112 | QVQLVQSGAEVKKPGASVKVSCKASGFNIKDYYMHWVRQAP |
| | GQGLEWMGWIDPENGDTEYAPKFQGRVTMTADTSTSTVYMEL |
| | SSLRSEDTAVYYCNFYDVYSEGALDYWGQGTLVTVSS |
| Seq Id No. 113 | GACATTCAGATGACTCAATCACCCTCGTCCCTCTCAGCTTCC |
| | GTCGGTGATAGGGTAACAATCACATGTCAAGCGAGCGAGAA |
| | CATCTATTTCTCGCTTGCGTGGTATCAGCAGAAGCCTGGGAA |
| | AGCGCCCAAGTTGCTGATCTACAATGCCAACAATTTGGAGGA |
| | TGGGGTGCCATCGAGATTTTCGGGATCCGGCAGCGGAACTG |
| | ACTTCACGTTCACCATTAGCTCGCTTCAGCCGGAGGACATTG |
| | CCACCTACTATTGCAAAGAAGCATACGATTCACCGTTTACGT |
| | TTGGACAGGGGACAAAGCTCGAAATCAAA |
| Seq Id No. 114 | DIQMTQSPSSLSASVGDRVTITCQASENIYFSLAWYQQKPGKAP |
| | KLLIYNANNLEDGVPSRFSGSGSGTDFTFTISSLQPEDIATYYCK |
| | EAYDSPFTFGQGTKLEIK |
| Seq Id No. 115 | CAAGTACAATTGGTGCAGTCAGGAGCAGAAGTCAAGAAGCC |
| | GGGTGCTAGCGTGAAAGTCAGCTGTAAGACTTCGGGATATA |
| | CTTTCACGAGCTACTGGATGCACTGGGTCCGCCAGGCCCCAG |
| | GCCAGGGGCTTGAGTGGATGGGTGCGCTGTACCCCGGAAAT |
| | TCGGTCACAAACTATAACCAGAAGTTCAAAGGGAGGGTGAC |
| | AATGACCGCGGACACGTCAACGTCCACTGTATACATGGAGCT |
| | GTCCTCGCTCAGATCAGAGGATACGGCGGTGTACTATTGCAC |
| | ACGGGGGTTTTTGACAGCCCCTTACTTTGACTCGTGGGGACA |
| | GGGGACCACCGTGACCGTCTCTAGT |
| Seq Id No. 116 | QVQLVQSGAEVKKPGASVKVSCKTSGYTFTSYWMHWVRQAP |
| | GQGLEWMGALYPGNSVTNYNQKFKGRVTMTADTSTSTVYME |
| | LSSLRSEDTAVYYCTRGFLTAPYFDSWGQGTTVTVSS |
| Seq Id No. 117 | GACATTCAGATGACTCAATCACCCTCGTCCCTCTCAGCTTCC |
| | GTCGGTGATAGGGTAACAATCACATGTCAAGCGAGCGAGAA |
| | CATCTATTTCTCGCTTGCGTGGTATCAGCAGAAGCCTGGGAA |
| | AGCGCCCAAGTTGCTGATCTACAATGCCAACAATTTGGAGGA |
| | TGGGGTGCCATCGAGATTTTCGGGATCCGGCAGCGGAACTG |
| | ACTACACGTTCACCATTAGCTCGCTTCAGCCGGAGGACATTG |
| | CCACCTACTTCTGCAAAGAAGCATACGATTCACCGTTTACGT |
| | TTGGACAGGGGACAAAGCTCGAAATCAAA |
| Seq Id No. 118 | DIQMTQSPSSLSASVGDRVTITCQASENIYFSLAWYQQKPGKAP |
| | KLLIYNANNLEDGVPSRFSGSGSGTDYTFTISSLQPEDIATYFCK |
| | EAYDSPFTFGQGTKLEIK |
| Seq Id No. 119 | CAAATCCAATTGGTGCAGTCAGGAGCAGAAGTCAAGAAGCC |
| | GGGTGCTAGCGTGAAAGTCAGCTGTAAGACTTCGGGATATA |
| | CTTTCACGAGCTACTGGATGCACTGGGTCCGCCAGGCCCCAG |
| | GCCAGGGGCTTGAGTGGATGGGTGCGCTGTACCCCGGAAAT |
| | TCGGTCACAAACTATAACCAGAAGTTCAAAGGGAGGGCCAA |
| | GCTGACCGCGGACACGTCAACGTCCACTGCCTACATGGAGCT |
| | GTCCTCGCTCAGATCAGAGGATACGGCGGTGTACTATTGCAC |
| | ACGGGGGTTTTTGACAGCCCCTTACTTTGACTCGTGGGGACA |
| | GGGGACCACCGTGACCGTCTCTAGT |
| Seq Id No. 120 | QIQLVQSGAEVKKPGASVKVSCKTSGYTFTSYWMHWVRQAPG |
| | QGLEWMGALYPGNSVTNYNQKFKGRAKLTADTSTSTAYMELS |
| | SLRSEDTAVYYCTRGFLTAPYFDSWGQGTTVTVSS |
| Seq Id No. 121 | GATATCGTAATGACGCAATCCCCGGACTCACTTGCCGTGTCG |
| | CTTGGCGAAAGAGCTACCATTAACTGCAAGTCATCCCAGTCA |
| | TTGTTGCATTCGTCGAACCAGAGGAATTACCTGGCGTGGTAC |
| | CAACAAAAGCCTGGACAGCCACCCAAATTGTTGATCTATTG |
| | GCGTCAATTCGCGAAAGCGGGGTCCCCGACCGTTTCTCGGG |
| | AAGCGGTTCCGGTACTGACTTTACACTCACGATCAGCTCGCT |
| | CCAGGCAGAGGATGTGGCGGTATACTATTGTCACCAGTATTA |
| | CTCATACTCGACATTCGGGCAGGGAACCAAACTGGAGATCA |
| | AA |
| Seq Id No. 122 | DIVMTQSPDSLAVSLGERATINCKSSQSLLHSSNQRNYLAWYQ |
| | QKPGQPPKLLIYWASIRESGVPDRFSGSGSGTDFTLTISSLQAED |
| | VAVYYCHQYYSYSTFGQGTKLEIK |
| Seq Id No. 123 | CAGGTCACACTTAAGGAGTCGGGTCCAGCGCTCGTGAAGCC |
| | CACACAGACCTTGACCCTCACGTGTACGTTCTCGGGAGCC |
| | ACTTACGACTAGCGGGATGGGCGTAAGCTGGATTCGGCAAC |
| | CTCCGGGGAAAGCGCTGGAATGGTTGGCACACATCTTCTGGG |
| | ATGATGACAAAAGGTATAACCCCTCGCTCACGTCGCGCCTGA |
| | CAATCTCAAAGGACACCTCCAAAAACCAGGTAGTGCTTACG |
| | ATGACGAATATGGATCCCGTGGACACAGCAACTTACTACTGC |
| | GCCAGAGGAGGAGATTACTATTCCACAGGGTTTGGTTTTGAC |
| | TACTGGGGGCAGGGAACTCTGGTCACCGTCTCTAGT |
| Seq Id No. 124 | QVTLKESGPALVKPTQTLTLTCTFSGFSLTTSGMGVSWIRQPPG |
| | KALEWLAHIFWDDDKRYNPSLTSRLTISKDTSKNQVVLTMTNM |
| | DPVDTATYYCARGGDYYSTGFGFDYWGQGTLVTVSS |
| Seq Id No. 125 | GATATCGTAATGACGCAATCCCCGGACTCACTTGCCGTGTCG |
| | CTTGGCGAAAGAGCTACCATTAACTGCAAGTCATCCCAGTCA |
| | TTGTTGCATTCGTCGAACCAGAGGAATTACCTGGCGTGGTAC |
| | CAACAAAAGCCTGGACAGCCACCCAAATTGTTGATCAGCTG |
| | GGCGTCAATTCGCGAAAGCGGGGTCCCCGACCGGTTCTCGG |
| | GAAGCGGTTCCGGTACTGACTTTACACTCACGATCAGCTCGC |
| | TCCAGGCAGAGGATGTGGCGGTATACTATTGTCACCAGTATT |
| | ACTCATACTCGACATTCGGGCAGGGAACCAAACTGGAGATC |
| | AAA |
| Seq Id No. 126 | DIVMTQSPDSLAVSLGERATINCKSSQSLLHSSNQRNYLAWYQ |
| | QKPGQPPKLLISWASIRESGVPDRFSGSGSGTDFTLTISSLQAED |
| | VAVYYCHQYYSYSTFGQGTKLEIK |
| Seq Id No. 127 | CAGGTCACACTTAAGGAGTCGGGTCCAGCGCTCGTGAAGCC |
| | CACACAGACCTTGACCCTCACGTGTACGTTCTCGGGATTTTC |
| | ACTTAGCACTAGCGGGATGGGCGTAAGCTGGATTCGGCAAC |
| | CTCCGGGGAAAGCGCTGGAATGGTTGGCACACATCTTCTGGG |
| | ATGATGACAAAAGGTATAACCCCTCGCTCACGTCGCGCCTGA |
| | CAATCTCAAAGGACACCTCCAAAAACCAGGTAGTGCTTACG |
| | ATGACGAATATGGATCCCGTGGACACAGCAACTTACTACTGC |
| | GCCAGAGGAGGAGATTACTATTCCACAGGGTTTGGTTTTGAC |
| | TACTGGGGGCAGGGAACTCTGGTCACCGTCTCTAGT |
| Seq Id No. 128 | QVTLKESGPALVKPTQTLTLTCTFSGFSLSTSGMGVSWIRQPPG |
| | KALEWLAHIFWDDDKRYNPSLTSRLTISKDTSKNQVVLTMTNM |
| | DPVDTATYYCARGGDYYSTGFGFDYWGQGTLVTVSS |

In yet other embodiments, the invention relates to a binding agent, such as an isolated antibody that exhibits a similar binding pattern to human WISE peptides in a “human WISE peptide epitope competition binding assay” as that exhibited by at least one of the antibodies Ab-AA, Ab-AB, Ab-AC, Ab-AD, Ab-AE, Ab-AF, Ab-AG, Ab-AH, Ab-AI, Ab-AJ and D14 or derivatives thereof; the isolated antibody, or an antigen-binding fragment thereof, may be a polyclonal antibody, a monoclonal antibody, a humanized antibody, a human antibody, or a chimeric antibody.

Examples of human antibodies specific for WISE are shown in Table 5 below.

TABLE 5

| | |
|---|---|
| Seq Id No. 129 | CAGTACGAATTGACTCAGCCACCCTCAGTGGCCGTGTCCCC |
| | TGGAAAGACAGCCAGCATCACCTGCGCTGGAGATGAATTGG |
| | GTAATAAATATGCTGCGTGGTACCAGCAGAAGCCAGGCCAG |
| | GCCCCTGTGCTGGTCGTCTATGATGATAGCGACCGGCCCTC |
| | AGGGATCCCTGAGCGATTCTCTGGCTCCAACTCTGGGAACA |
| | CGGCCACCCTGACCATCAGCAGGGTCGAAGCCGGGGATGA |
| | GGCCGACTATTACTGTCAGGTGTGGGATAGAAGTAGTTATC |
| | ATGTGGTATTCGGCGGAGGGACCAAGCTGACCGTCCTAGGT |
| | CAGCCCAAGGCCAACCCCACTGTCACTCTGTTCCCGCCCTCC |
| | TCTGAGGAGCTCCAAGCCAACAAGGCCACACTAGTGTGTCT |
| | GATCAGTGACTTCTACCCGGGAGCTGTGACAGTGGCCTGGA |
| | AGGCAGATGGCAGCCCCGTCAAGGCGGGAGTGGAGACCAC |
| | CAAACCCTCCAAACAGAGCAACAACAAGTACGCGGCCAGC |
| | AGCTACCTGAGCCTGACGCCCGAGCAGTGGAAGTCCCACAG |
| | AAGCTACAGCTGCCAGGTCACGCATGAAGGGAGCACCGTG |
| | GAGAAGACAGTGGCCCCTACAGAATGTTCA |
| Seq Id No. 130 | QYELTQPPSVAVSPGKTASITCAGDELGNKYAAWYQQKPGQA |
| | PVLVVYDDSDRPSGIPERFSGSNSGNTATLTISRVEAGDEADYY |
| | CQVWDRSSYHVVFGGGTKLTVLGQPKANPTVTLFPPSSEELQA |
| | NKATLVCLISDFYPGAVTVAWKADGSPVKAGVETTKPSKQSN |
| | NKYAASSYLSLTPEQWKSHRSYSCQVTHEGSTVEKTVAPTECS |
| Seq Id No. 131 | GAAGTTCAATTGTTAGAGTCTGGTGGCGGTCTTGTTCAGCCT |
| | GGTGGITCTTTACGTCTTTCTTGCGCTGCTTCCGGATTCACTT |
| | TCTCTCTTTACACTATGCAGTGGGTTCGCCAAGCTCCTGGTA |
| | AAGGTTTGGAGTGGGTTTCTGGTATCGGTTCTTCTGGTGGCG |
| | GTACTTCTTATGCTGACTCCGTTAAAGGTCGCTTCACTATCT |
| | CTAGAGACAACTCTAAGAATACTCTCTACTTGCAGATGAAC |
| | AGCTTAAGGGCTGAGGACACTGCCGTGTATTACTGTGCGAG |
| | AGGGGTCAGCAGTTGGITTTTCGAGTACTGGGGCCAGGGAA |
| | CCCTGGTCACCGTCTCTAGTGCCTCCACCAAGGGCCCATCG |
| | GTCTTCCCCCTGGCGCCCTGCTCCAGGAGCACCTCCGAGAG |
| | CACAGCGGCCCTGGGCTGCCTGGTCAAGGACTACTTCCCCG |
| | AACCGGTGACGGTGTCGTGGAACTCAGGCGCTCTGACCAGC |
| | GGCGTGCACACCTTCCCAGCTGTCCTACAGTCCTCAGGACTC |
| | TACTCCCTCAGCAGCGTGGTGACCGTGCCCTCCAGCAACTTC |
| | GGCACCCAGACCTACACCTGCAACGTAGATCACAAGCCCAG |
| | CAACACCAAGGTGGACAAGACAGTTGAGCGCAAATGTTGTG |
| | TCGAGTGCCCACCGTGCCCAGCACCACCTGTGGCAGGACCG |
| | TCAGTCTTCCTCTTCCCCCCAAAACCCAAGGACACCCTCATG |
| | ATCTCCCGGACCCCTGAGGTCACGTGCGTGGTGGTGGACGT |
| | GAGCCACGAAGACCCCGAGGTCCAGTTCAACTGGTACGTGG |
| | ACGGCGTGGAGGTGCATAATGCCAAGACAAAGCCACGGGA |
| | GGAGCAGTTCAACAGCACGTTCCGTGTGGTCAGCGTCCTCA |
| | CCGTTGTGCACCAGGACTGGCTGAACGGCAAGGAGTACAAG |
| | TGCAAGGTCTCCAACAAAGGCCTCCCAGCCCCCATCGAGAA |
| | AACCATCTCCAAAACCAAAGGGCAGCCCCGAGAACCACAG |
| | GTGTACACCCTGCCCCCATCCCGGGAGGAGATGACCAAGAA |
| | CCAGGTCAGCCTGACCTGCCTGGTCAAAGGCTTCTACCCCA |
| | GCGACATCGCCGTGGAGTGGGAGAGCAATGGGCAGCCGGA |
| | GAACAACTACAAGACCACACCTCCCATGCTGGACTCCGACG |
| | GCTCCTTCTTCCTCTACAGCAAGCTCACCGTGGACAAGAGC |
| | AGGTGGCAGCAGGGGAACGTCTTCTCATGCTCCGTGATGCA |
| | TGAGGCTCTGCACAACCACTACACGCAGAAGAGCCTCTCCC |
| | TGTCTCCGGGTAAA |
| Seq Id No. 132 | EVQLLESGGGLVQPGGSLRLSCAASGFTFSLYTMQWVRQAPG |
| | KGLEWVSGIGSSGGGTSYADSVKGRFTISRDNSKNTLYLQMNS |
| | LRAEDTAVYYCARGVSSWFFEYWGQGTLVTVSSASTKGPSVF |
| | PLAPCSRSTSESTAALGCLVKDYFPEPVTVSWNSGALTSGVHT |
| | FPAVLQSSGLYSLSSVVTVPSSNFGTQTYTCNVDHKPSNTKVD |
| | KTVERKCCVECPPCPAPPVAGPSVFLFPPKPKDTLMISRTPEVT |
| | CVVVDVSHEDPEVQFNWYVDGVEVHNAKTKPREEQFNSTFR |
| | VVSVLTVVHQDWLNGKEYKCKVSNKGLPAPIEKTISKTKGQP |
| | REPQVYTLPPSREEMTKNQVSLTCLVKGFYPSDIAVEWESNGQ |
| | PENNYKTTPPMLDSDGSFFLYSKLTVDKSRWQQGNVFSCSVM |
| | HEALHNHYTQKSLSLSPGK |
| Seq Id No. 133 | AGDELGNKYAA |
| Seq Id No. 134 | DDSDRPS |
| Seq Id No. 135 | QVWDRSSYHVV |
| Seq Id No. 136 | LYTMQ |
| Seq Id No. 137 | GIGSSGGGTSYADSVKG |
| Seq Id No. 138 | GVSSWFFEY |

The human antibodies in table five can be further mutated in their CDR's to enhance antibody function, for example, by increasing affinity to human WISE with mutations described in Table 6 below. Numbering is based on the amino acid residue position in heavy chain or light chain of the antibody (Seq Id No. 132 or Seq Id No. 130).

TABLE 6

| | Mutations |
|---|---|
| DNW-D14 | parental |
| D14-DM03 | H35(HC CDR2-5D) + L33(LC CDR1-7E) |
| D14-DM05 | H35(HC CDR2-5D) + L36(LC CDR1-7S) |
| D14-DM09 | H35(HC CDR2-5D) + L98(LC CDR3-5W) |
| D14-DM13 | H66(HC CDR2-8P) + L33(LC CDR1-7E) |
| D14-DM15 | H66(HC CDR2-8P) + L36(LC CDR1-7S) |
| D14-DM16 | H66(HC CDR2-8P) + L37(LC CDR1-7T) |
| D14-DM19 | H66(HC CDR2-8P) + L98(LC CDR3-5W) |
| D14-DM25 | HI 18(HC CDR2-17H) + L36(LC CDR1-7S) |
| D14-DM44 | H95(HC CDR2-14H) + L36(LC CDR1-7S) |
| D14-DM46 | H41(HC CDR2-5Q) + L36(LC CDR1-7S) |
| D14-TM1 | H66(HC CDR2-8P) + H127(HC CDR3-4G) + |
| | L34(LC CDR1-7G) |
| D14-TM2 | H66(HC CDR2-8P) + H127(HC CDR3-4G) + |
| | L36(LC CDR1-7S) |
| D14-L36 | LC-CDR1-7 N−>S |

The invention still further relates to a method for treating a renal and/or lung fibrotic disease or disorder in a mammalian subject which comprises providing to a subject in need of such treatment an amount of an anti-WISE binding agent sufficient to decrease symptoms associated with the disorder, wherein the anti-WISE binding agent comprises an antibody, or WISE-binding fragment thereof.

Provided herein are antibodies that specifically bind to human WISE. The antibodies of the invention are characterized by their ability to cross-block the binding of at least one antibody disclosed herein to human WISE and/or to be cross-blocked from binding human WISE by at least one antibody disclosed herein. The invention also provides an isolated antibody, or an antigen-binding fragment thereof, that can block the effect of WISE in a cell based assay. The invention also provides an monoclonal antibody or fragment thereof that can activate the effect of WISE in a cell based assay.

These and other aspects of the present invention will become apparent upon reference to the following detailed description and attached drawings. All references disclosed herein are hereby incorporated by reference in their entireties as if each was incorporated individually.

### BRIEF DESCRIPTION OF THE DRAWINGS

FIG. 1: Identification of antibodies requiring Loop-2 for binding to WISE.

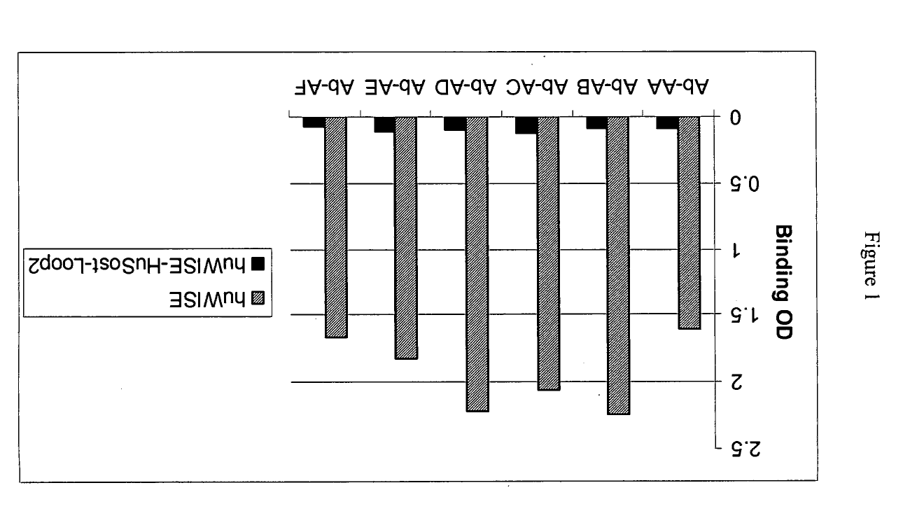

FIG. 2: Identifying neutralizing Loop-2 Antibodies in a cell-based assay. Concentrations of proteins are: 0.3 ug/ml hWISE and 3 ug/ml Ab.

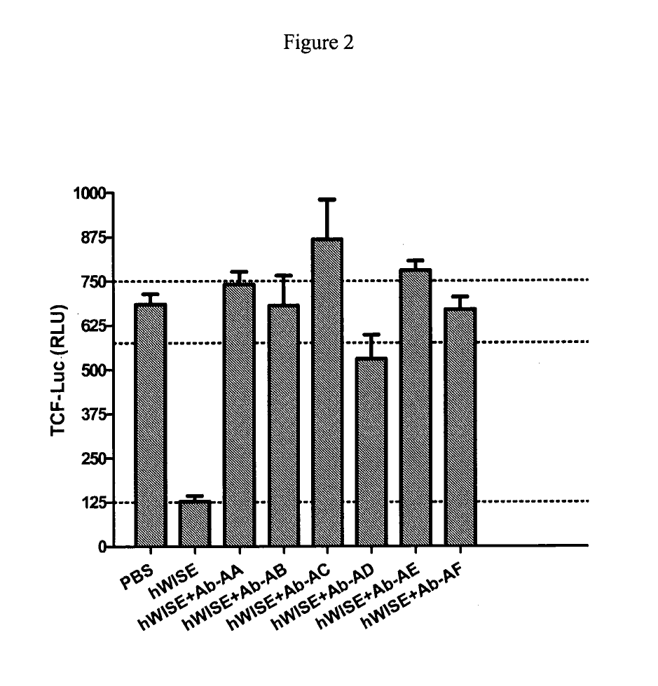

FIG. 3: Neutralizing Anti-Wise Loop2 Antibody Inhibits Wise-Lrp6 Binding.

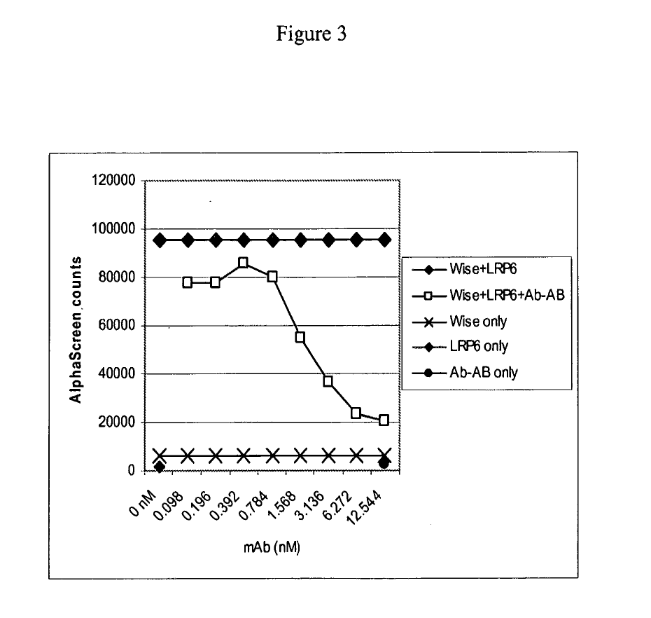

FIG. 4: Assay for measuring competitive binding of Ab-AA to hWISE. Ab-AA is bound to the plate. Ab-C, Ab-T, Ab-S, and Ab-P are previously identified control antibodies and are included in this and the following figures.

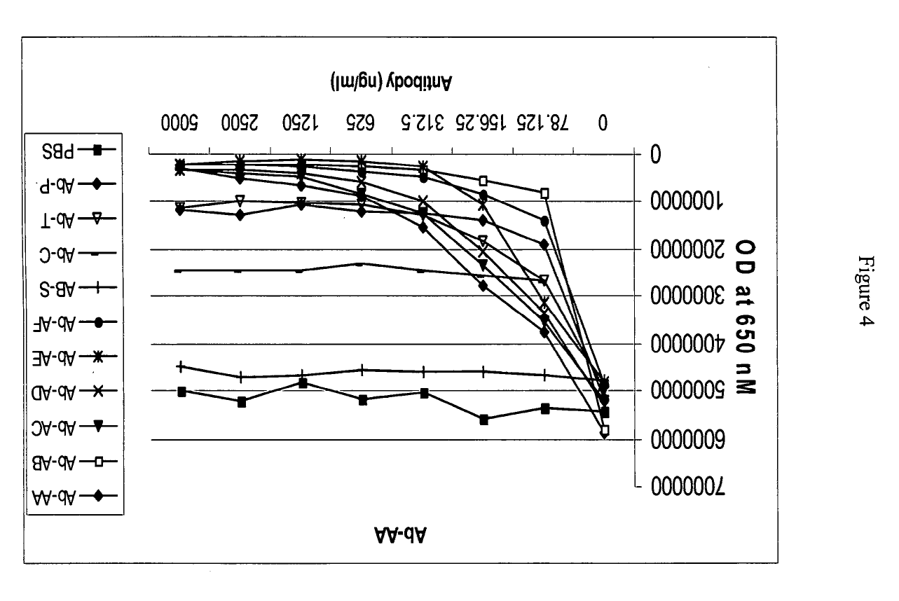

FIG. 5: Assay for measuring competitive binding of Ab-AB to hWISE. Ab-AB is bound to the plate.

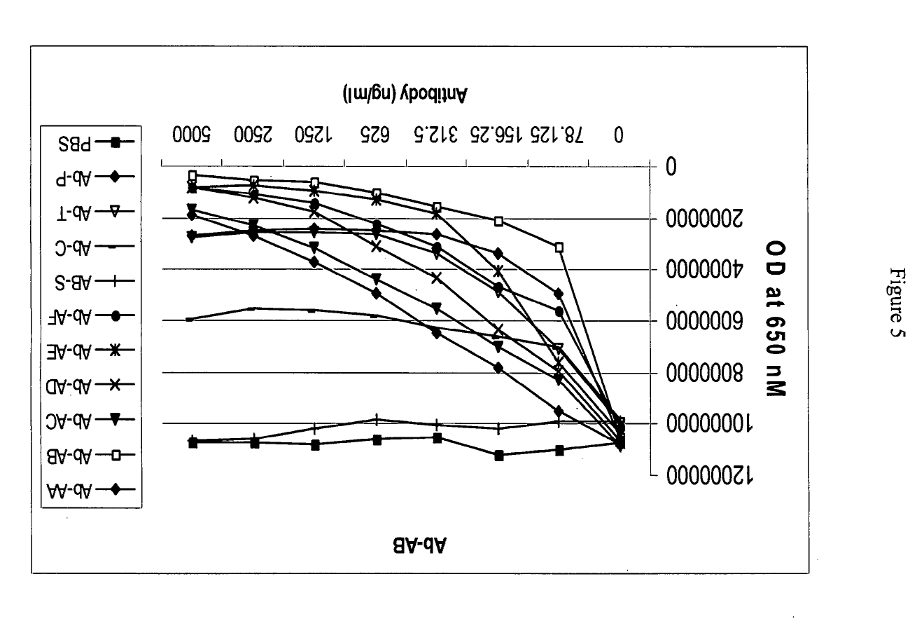

FIG. 6: Assay for measuring competitive binding of Ab-AC to hWISE. Ab-AC is bound to the plate.

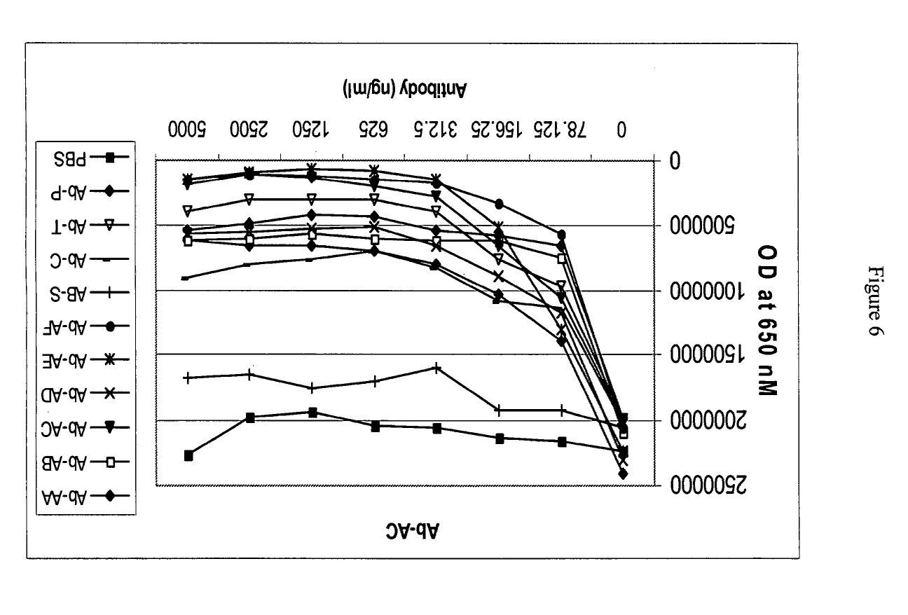

FIG. 7: Assay for measuring competitive binding of Ab-AD to hWISE. Ab-AD is bound to the plate.

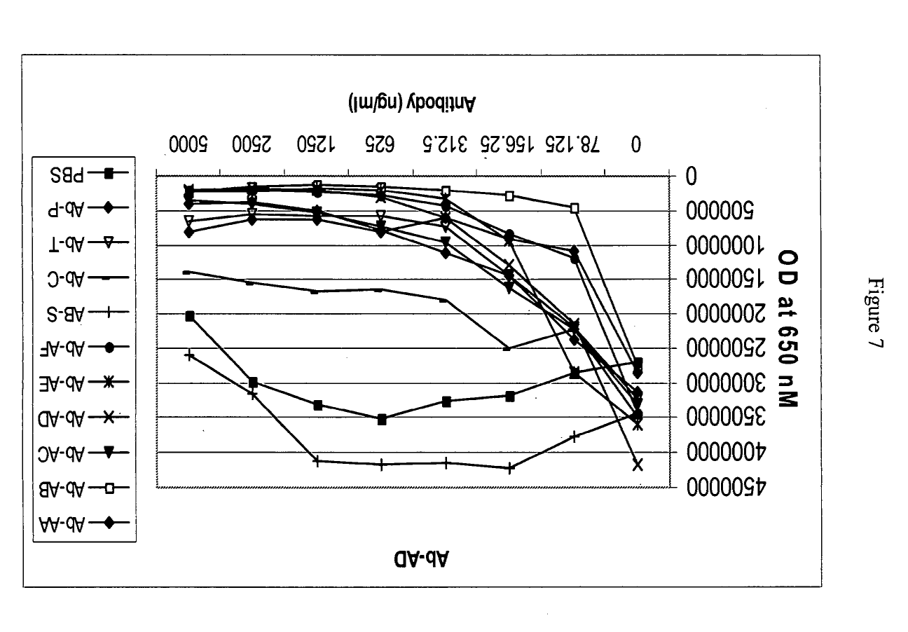

FIG. 8: Assay for measuring competitive binding of Ab-AE to hWISE. Ab-AE is bound to the plate.

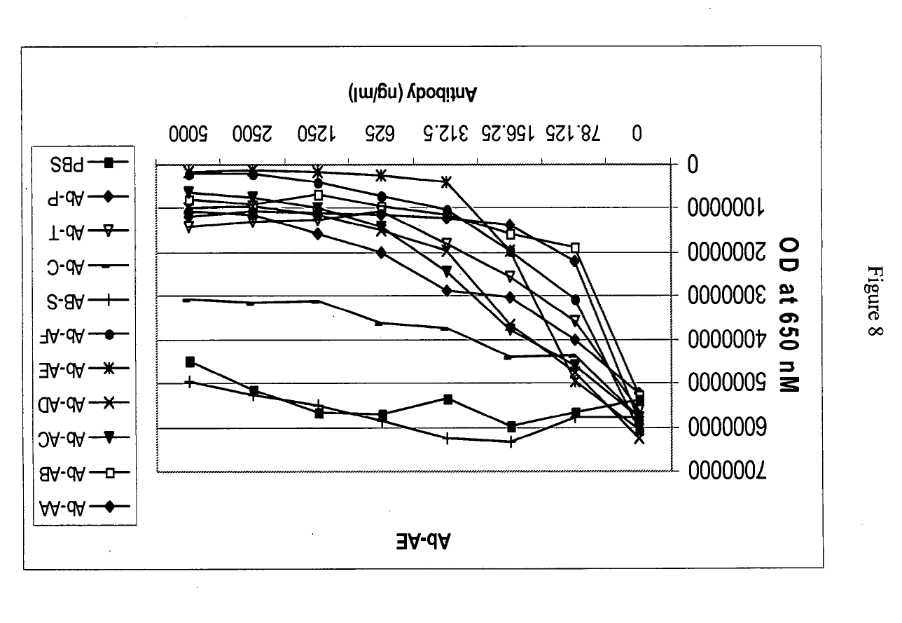

FIG. 9: Assay for measuring competitive binding of Ab-AF to hWISE. Ab-AF is bound to the plate.

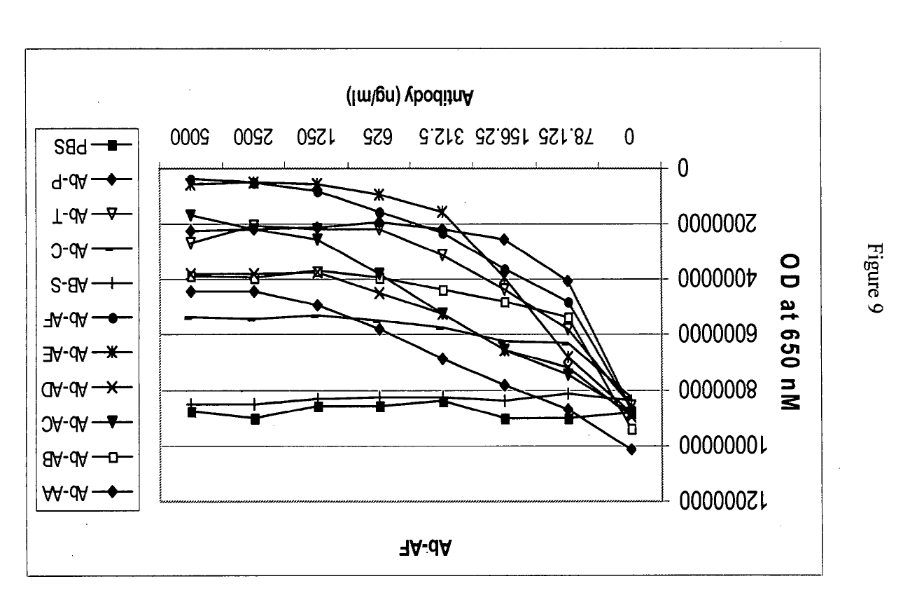

FIG. 10: Assay for measuring competitive binding of Ab-S to hWISE. Ab-S is bound to the plate.

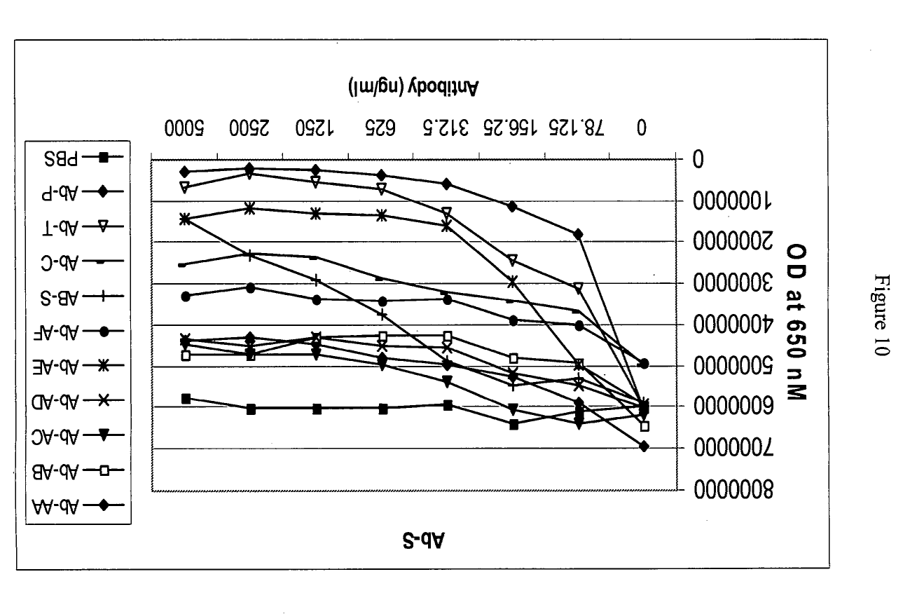

FIG. 11: Assay for measuring competitive binding of Ab- to hWISE. Ab-S is bound to the plate.

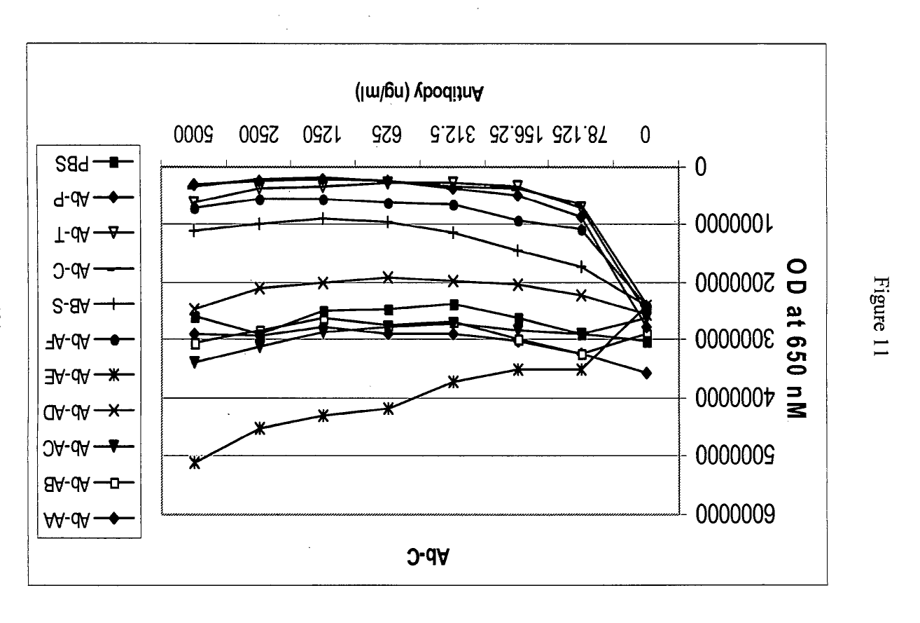

FIG. 12: Assay for measuring competitive binding of Ab-S to hWISE. Ab-S is bound to the plate.

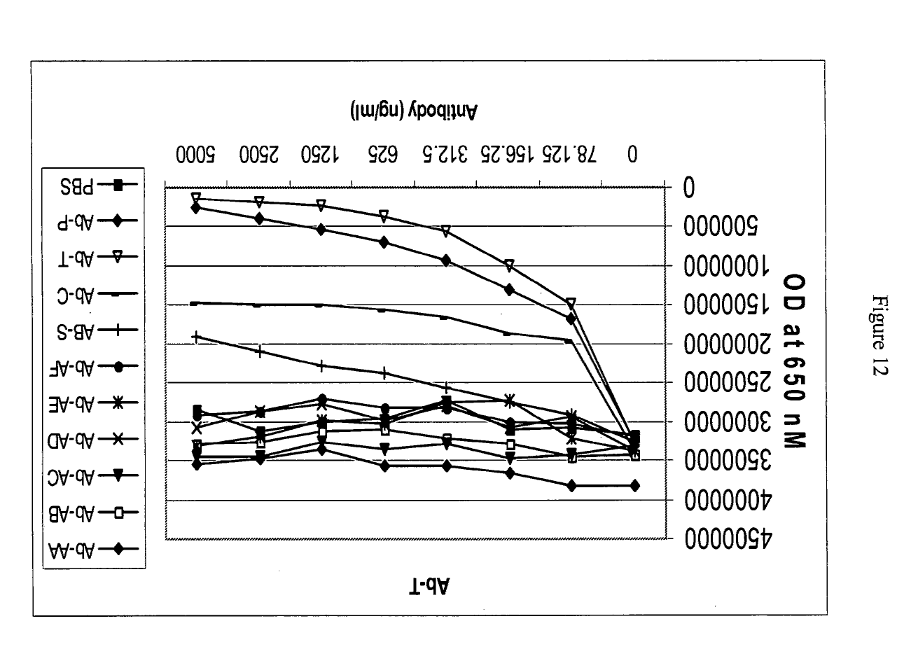

FIG. 13: Assay for measuring competitive binding of Ab-S to hWISE. Ab-S is bound to the plate.

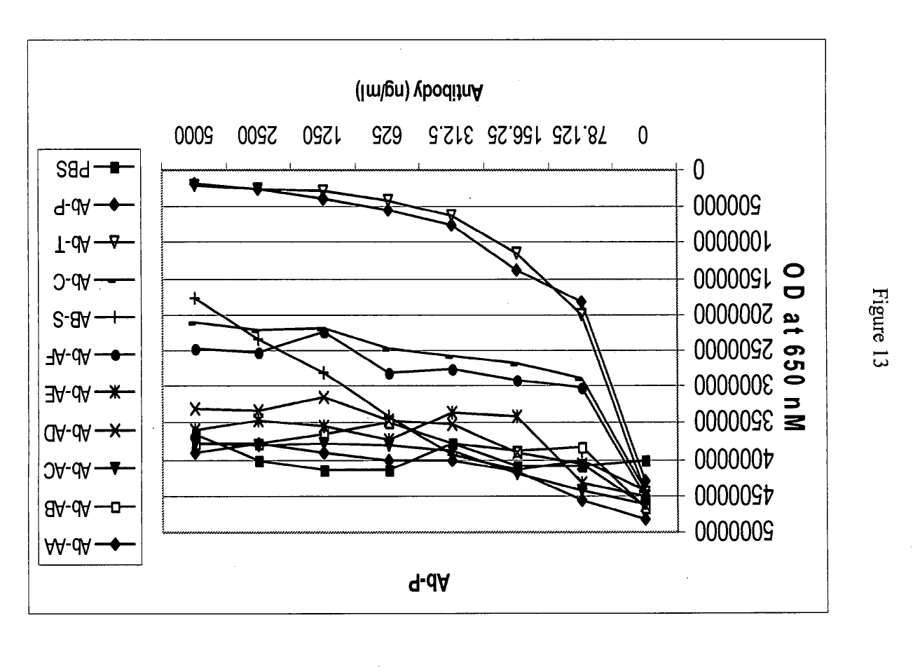

FIG. 14: Binding assay showing relative binding of hWISE antibodies to mutant hWISE where residues in the loop 2 have been individually mutated to alanine. Numbers less than one indicate reduced binding. HuWISE-HuSost-loop 2 is a control polypeptide that is a chimeric of hWISE and has HuSost loop 2 (see Examples). ‘WT residue’ indicates the amino acid that has been converted to alanine. These residues correspond with the amino acids 4 to 25 in Seq Id No: 9, respectively.

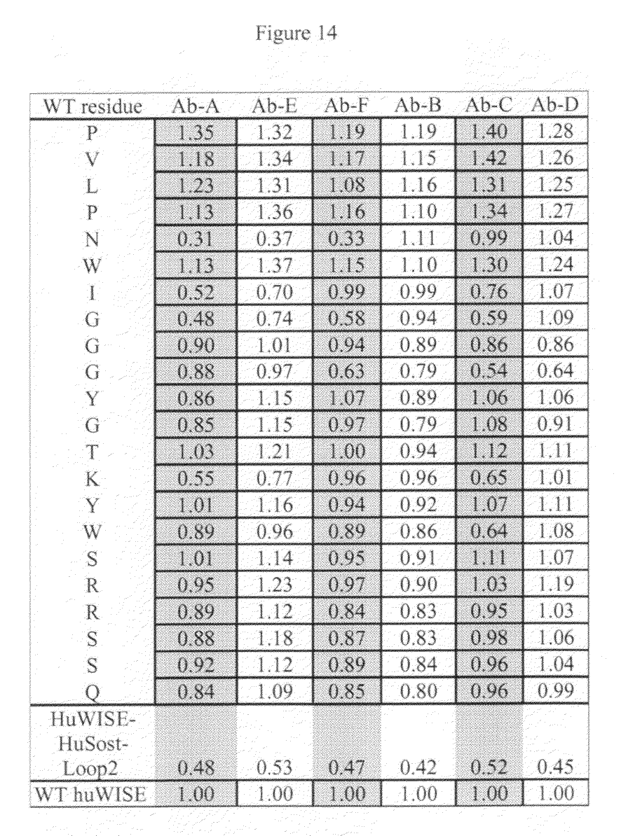

FIG. 15: WISE antibody treatment preserved renal function in T2DN model.

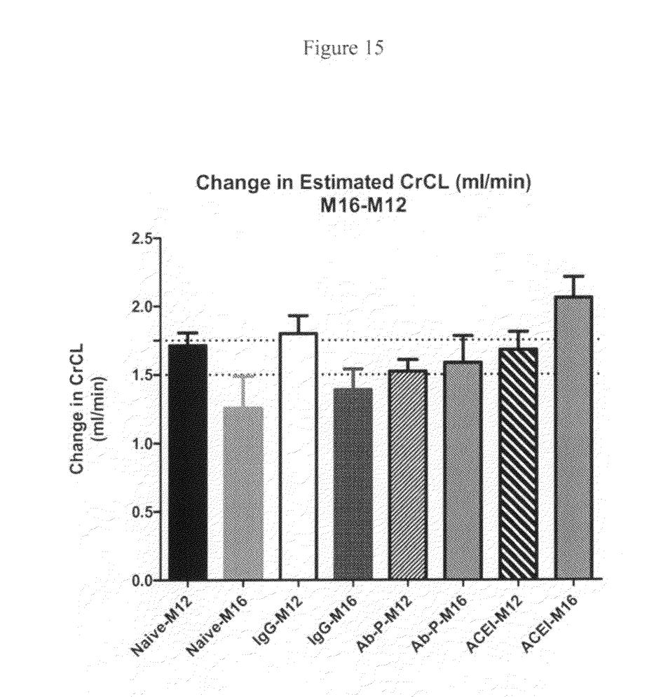

FIG. 16: WISE antibody treatment preserved renal function in T2DN model.

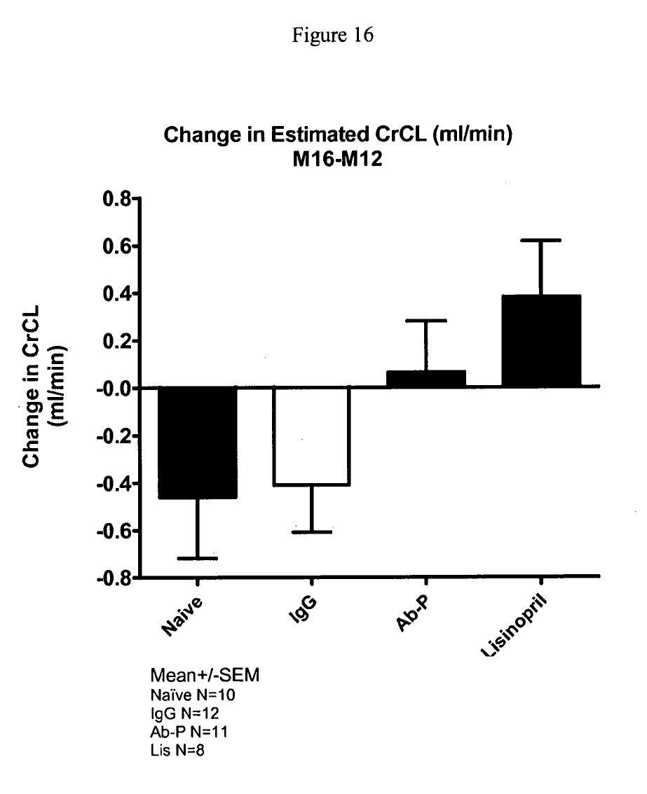

FIG. 17: WISE antibody treatment inhibited tubular-interstitial injury in T2DN model.

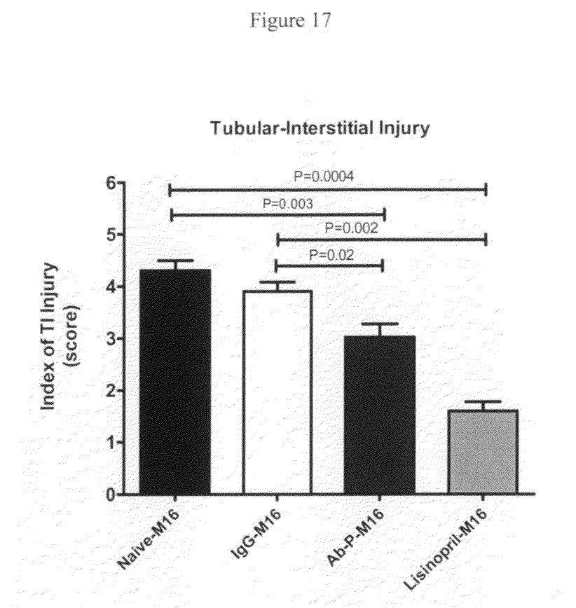

FIG. 18: WISE antibody treatment inhibited glomerular injury in T2DN model.

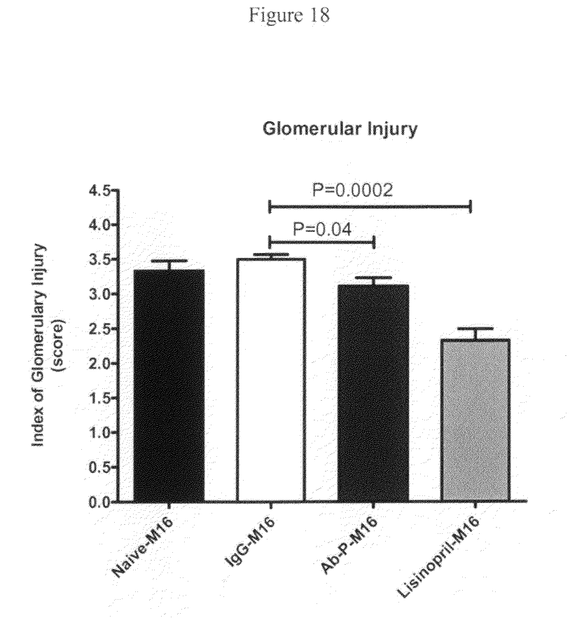

FIG. 19: WISE antibody treatment had no impact on established proteinuria in T2DN model.

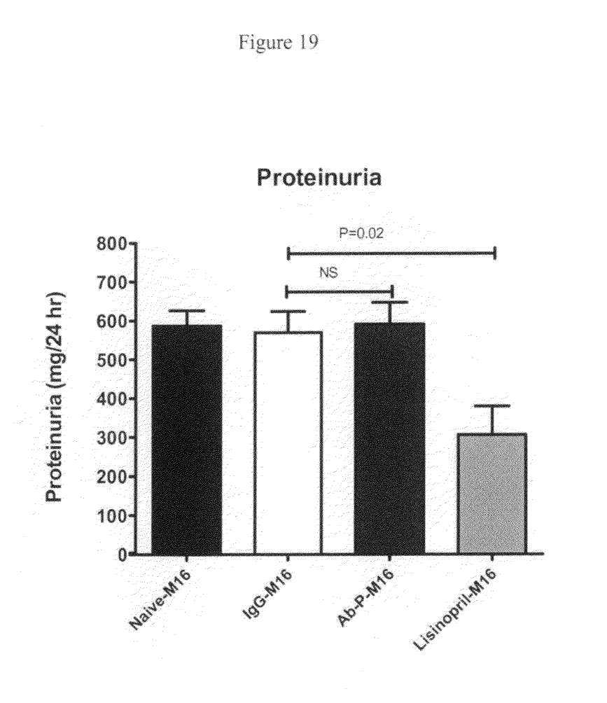

FIG. 20: Binding Profile of WISE Loop 2 Mab (Ab-AB) against WISE Mutants.

FIG. 21: Binding Profile of WISE Loop 2 Mab (Ab-AE) against WISE Mutants.

FIG. 22: Binding Profile of WISE Loop 2 Mab (Ab-AG) against WISE Mutants.

FIG. 23: Binding Profile of WISE Loop 2 Mab (Ab-AI) against WISE Mutants.

FIG. 24: Binding Profile of WISE Loop 2 Mab (Ab-AC) against WISE Mutants.

FIG. 25: Binding Profile of WISE Loop 2 Mab (Ab-AA) against WISE Mutants.

FIG. 26: Binding Profile of WISE Loop 2 Mab (Ab-AH) against WISE Mutants.

FIG. 27: Binding Profile of WISE Loop 2 Mab (Ab-AJ) against WISE Mutants.

FIG. 28: Binding Profile of WISE Loop 2 Mab (Ab-AF) against WISE Mutants.

FIG. 29: MC3T3E1-STF Expression of humanized WISE loop2 mabs showing activity of antibodies.

### DETAILED DESCRIPTION

The present invention relates in part to regions of the WISE protein that contain epitopes recognized by antibodies, where these antibodies are also capable of binding to the epitopes in the context of a full-length WISE polypeptide, and methods of making and using these epitopes. The invention also provides binding agents (such as antibodies) that specifically bind to WISE or portions of WISE, and methods for using such binding agents. The binding agents are useful to block or impair the binding of human WISE to one or more ligand(s) and its biological activity.

As used herein, the term human WISE is intended to include the protein depicted in Seq Id No. 2, 4, 6, and 8 and allelic variants thereof. Orthologs of WISE are also described and include mouse, rat and cynomolgus Seq Id No. 2, 4, 6, and 8. WISE can be purified from host cells that have been transfected by a gene encoding WISE by elution of filtered supernatant of host cell culture fluid. The preparation and further purification are described in the Examples. Human WISE nucleic acids are described in U.S. Pat. No. 5,780,263.

It will be understood by one of skill in the art that there is a high degree of sequence identity between the orthologs of WISE. Accordingly, binding agents to human WISE will be expected to bind to the mouse, rat or cynomolgus WISE in cases where the recognition site of the binding agent, e.g., an antibody binding site such as an epitope, is highly conserved and in particular nearly or completely identical to the human sequence. Thus, when the term “specific binding to WISE” is used, it is understood to include binding to multiple species of WISE where the sequences between species are conserved.

Examples of binding agents according to the invention include the following: Ab-AA, Ab-AB, Ab-AC, Ab-AD, Ab-AE, Ab-AF, Ab-AG, Ab-AH, Ab-AI, Ab-AJ, and D14 and also an antibody comprising Seq Id Nos.: 34, 35, and 36, and Seq Id Nos.: 37, 38 and 39; Seq Id Nos.: 40, 41 and 42, and Seq Id Nos.: 43, 44 and 45; Seq Id Nos.: 46, 47 and 48 and Seq Id Nos.: 49, 50 and 51; Seq Id Nos.: 52, 53 and 54 and Seq Id Nos.: 55, 56 and 57; Seq Id Nos.: 58, 59 and 60 and Seq Id Nos.: 61, 62 and 63; Seq Id Nos.: 64, 65 and 66, and Seq Id Nos.: 67, 68 and 69; Seq Id Nos.: 86, 87 and 88 and Seq Id Nos.: 89, 90 and 69; Seq Id Nos.: 91, 92 and 93 and Seq Id Nos.: 94, 95 and 96; Seq Id Nos.: 97, 98 and 99 and Seq Id Nos.: 100, 101 and 102; Seq Id Nos.: 103, 104 and 105 and Seq Id Nos.: 106, 107 and 108 and Seq Id Nos.: 133, 134 and 135 and Seq Id Nos.: 136, 137 and 138. Furthermore, examples of CDR-L1 polypeptides of the invention include those shown in Seq Id Nos.: 34, 40, 46, 52, 58, 64, 86, 91, 97, 103 and 133. Examples of CDR-L2 include those shown in Seq Id Nos.: 35, 41, 47, 53, 59, 65, 87, 92, 98, 104 and 134. Examples of CDR-L3 include those shown in Seq Id Nos.: 36, 42, 48, 54, 60, 66, 88, 93, 99, 105, and 135. Examples of CDR-H1 include those shown in Seq Id Nos.: 37, 43, 49, 55, 61, 67, 89, 94, 100, 106 and 136. Examples of CDR-H2 include those shown in Seq Id Nos.: 38, 44, 50, 56, 62, 68, 90, 95, 101, 107 and 137. Examples of CDR-H3 include those shown in Seq Id Nos.: 39, 45, 51, 57, 63, 69, 96, 102, 108, and 138.

As used herein, Ab-AA is comprised of the polypeptides expressed by the nucleotides shown in Seq Id No. 10 and 12; Ab-AB is comprised of the polypeptide expressed by the nucleotides shown in Seq Id No. 14 and 16; Ab-AC is comprised of the polypeptides expressed by the nucleotides shown in Seq Id No. 18 and 20; Ab-AD is comprised of the polypeptides expressed by the nucleotides shown in Seq Id No. 22 and 24; Ab-AE is comprised of the polypeptides expressed by the nucleotides shown in Seq Id No. 26 and 28; Ab-AF is comprised of the polypeptides expressed by the nucleotides shown in Seq Id No. 30 and 32; Ab-AG is comprised of the polypeptides expressed by the nucleotides shown in Seq Id No. 70 and 72; Ab-AH is comprised of the polypeptides expressed by the nucleotides shown in Seq Id No. 74 and 76; Ab-AI is comprised of the polypeptides expressed by the nucleotides shown in Seq Id No. 78 and 80; Ab-AJ is comprised of the polypeptides expressed by the nucleotides shown in Seq Id No. 82 and 84; and D14 is comprised of the polypeptides expressed by nucleotides shown in Seq Id Nos.: 129 and 131. It is also understood that each of Ab-AA, Ab-AB, Ab-AC, Ab-AD, Ab-AE, Ab-AF, Ab-AG, Ab-AH, Ab-AI, Ab-AJ, and D14 shown herein lack signal peptides and one of skill in the art could express each of these polypeptides using conventional techniques including use of signal peptides known in the art. It is also understood that each of Ab-AA, Ab-AB, Ab-AC, Ab-AD, Ab-AE, Ab-AF, Ab-AG, Ab-AH, Ab-AI, and Ab-AJ described herein include only the variable light and heavy chains and do not include the constant regions.

Furthermore, the non-human antibodies described herein can be humanized by a number of different strategies including the simple ‘cut and paste’ of the CDR's or can also include back mutations to preserve key murine sequences in the humanized molecule. Specific examples of humanization include Ab-AB, Ab-AI and Ab-AE in the examples below (Seq Id Nos.: 114 and 116, Seq Id Nos.: 118 and 120, Seq Id Nos.: 122 and 124, Seq Id Nos.: 126 and 128, and Seq Id Nos. 110 and 112).

Additional antibody data is presented herein with reference to previously disclosed molecules (e.g., Ab-C, Ab-T, Ab-S, and Ab-P) in U.S. patent application Ser. No. 12/275,850 (US 2009/0130114) which is incorporated herein by reference in its entirety.

Binding agents of the invention are typically antibodies or fragments thereof, as defined herein. The term “antibody” refers to an intact antibody, or a binding fragment thereof. An antibody may comprise a complete antibody molecule (including polyclonal, monoclonal, chimeric, humanized, or human versions having full length heavy and/or light chains), or comprise an antigen binding fragment thereof. Antibody fragments include F(ab′)2, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv (See e.g., Hollinger and Hudson, 2005, Nature. Biotechnology, 23, 9, 1126-1136). Antibody polypeptides are also disclosed in U.S. Pat. No. 6,703,199, including fibronectin polypeptide monobodies. Other antibody polypeptides are disclosed in U.S. patent Publication 2005/0238646, which are single-chain polypeptides. As used herein, the isolated antibody or an antigen-binding fragment thereof may be a polyclonal antibody, a monoclonal antibody, a humanized antibody, a human antibody, a chimeric antibody or the like.

Antigen binding fragments derived from an antibody can be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)2. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fc fragment directly. These methods are described, for example, by Goldenberg, U.S. Pat. No. 4,331,647, Nisonoff et al., Arch. Biochem. Biophys. 89:230, 1960; Porter, Biochem. J. 73:119, 1959; Edelman et al., in Methods in Enzymology 1:422 (Academic Press 1967); and by Andrews, S. M. and Titus, J. A. in Current Protocols in Immunology (Coligan J. E., et al., eds), John Wiley & Sons, New York (2003). pages 2.8.1-2.8.10 and 2.10A.1-2.10A.5. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

An antibody fragment may also be any synthetic or genetically engineered protein. For example, antibody fragments include isolated fragments consisting of the light chain variable region, “Fv” fragments consisting of the variable regions of the heavy and light chains, recombinant single chain polypeptide molecules in which light and heavy variable regions are connected by a peptide linker (scFv proteins).

Another form of an antibody fragment is a polypeptide comprising one or more complementarity determining regions (CDRs) of an antibody. CDRs (also termed “minimal recognition units”, or “hypervariable region”) can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology 2:106, 1991; Courtenay-Luck, “Genetic Manipulation of Monoclonal Antibodies,” in Monoclonal Antibodies. Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)).

Thus, in one embodiment, the binding agent comprises at least one CDR as described herein. The binding agent may comprise at least two, three, four, five or six CDR's as described herein. The binding agent further may comprise at least one variable region domain of an antibody described herein. The variable region domain may be of any size or amino acid composition and will generally comprise at least one CDR sequence responsible for binding to human WISE, for example CDR-H1, CDR-H2, CDR-H3 and/or the light chain CDRs specifically described herein and which is adjacent to or in frame with one or more framework sequences. In general terms, the variable (V) region domain may be any suitable arrangement of immunoglobulin heavy (VH) and/or light (VL) chain variable domains. Thus, for example, the V region domain may be monomeric and be a VH or VL domain, which is capable of independently binding human WISE with an affinity at least equal to 1×10⁻⁷M or less as described below. Alternatively, the V region domain may be dimeric and contain VH-VH, VH-VL, or VL-VL, dimers. The V region dimer comprises at least one VH and at least one VL chain that may be non-covalently associated (hereinafter referred to as FV). If desired, the chains may be covalently coupled either directly, for example via a disulfide bond between the two variable domains, or through a linker, for example a peptide linker, to form a single chain Fv (scFV).

The variable region domain may be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C-terminal amino acid to at least one other antibody domain or a fragment thereof. Thus, for example, a VH domain that is present in the variable region domain may be linked to an immunoglobulin CH1 domain, or a fragment thereof. Similarly a VL domain may be linked to a CK domain or a fragment thereof. In this way, for example, the antibody may be a Fab fragment wherein the antigen binding domain contains associated VH and VL domains covalently linked at their C-termini to a CHI and CK domain, respectively. The CH1 domain may be extended with further amino acids, for example to provide a hinge region or a portion of a hinge region domain as found in a Fab′ fragment, or to provide further domains, such as antibody CH2 and CH3 domains.

As described herein, binding agents comprise at least one of these CDRs. For example, one or more CDR may be incorporated into known antibody framework regions (IgG1, IgG2, etc.), or conjugated to a suitable vehicle to enhance the half-life thereof. Suitable vehicles include, but are not limited to Fc, polyethylene glycol (PEG), albumin, transferrin, and the like. These and other suitable vehicles are known in the art. Such conjugated CDR peptides may be in monomeric, dimeric, tetrameric, or other form. In one embodiment, one or more water-soluble polymer is bonded at one or more specific position, for example at the amino terminus, of a binding agent.

In certain embodiments, a binding agent comprises one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative binding agent comprises one or more of monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. In certain embodiments, one or more water-soluble polymer is randomly attached to one or more side chains. In certain embodiments, PEG can act to improve the therapeutic capacity for a binding agent, such as an antibody. Certain such methods are discussed, for example, in U.S. Pat. No. 6,133,426, which is hereby incorporated by reference for any purpose.

It will be appreciated by one of skill in the art that a binding agent of the present invention may have at least one amino acid substitution, providing that the binding agent retains binding specificity. Therefore, modifications to the binding agent structures are encompassed within the scope of the invention. These may include amino acid substitutions, which may be conservative or non-conservative and that do not destroy the WISE binding capability of a binding agent. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties. A conservative amino acid substitution may also involve a substitution of a native amino acid residue with a normative residue such that there is little or no effect on the polarity or charge of the amino acid residue at that position.

Non-conservative substitutions may involve the exchange of a member of one class of amino acids or amino acid mimetics for a member from another class with different physical properties (e.g. size, polarity, hydrophobicity, charge). Such substituted residues may be introduced into regions of the human antibody that are homologous with non-human antibodies, or into the non-homologous regions of the molecule.

Moreover, one skilled in the art may generate test variants containing a single amino acid substitution at each desired amino acid residue. Such testing can be done on the target of the binding agent as described below in the examples or on the therapeutic binding agent of the invention. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change may be avoided. In other words, based on information gathered from such routine experiments, one skilled in the art can readily determine the amino acids where further substitutions should be avoided either alone or in combination with other mutations.

A skilled artisan will be able to determine suitable variants of the polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides. In certain embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues which are important for activity or structure in similar proteins. One skilled in the art may opt for chemically similar amino acid substitutions for such predicted important amino acid residues.

A number of scientific publications have been devoted to the prediction of secondary structure. See Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol., 47: 45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will become dramatically more accurate.

Additional methods of predicting secondary structure include “threading” (Jones, D., Curr. Opin. Struct. Biol., 7(3):377-87 (1997); Sippl et al., Structure, 4(1):15-19 (1996)), “profile analysis” (Bowie et al., Science, 253:164-170 (1991); Gribskov et al., Meth. Enzym., 183:146-159 (1990); Gribskov et al., Proc. Nat. Acad. Sci., 84(13):4355-4358 (1987)), and “evolutionary linkage” (See Holm, supra (1999), and Brenner, supra (1997)).

In certain embodiments, variants of binding agents include glycosylation variants wherein the number and/or type of glycosylation site has been altered compared to the amino acid sequences of a parent polypeptide. In certain embodiments, variants comprise a greater or a lesser number of N-linked glycosylation sites than the native protein. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to WISE, or to increase or decrease the affinity of the antibodies to WISE described herein.

According to certain embodiments, preferred amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

In certain embodiments, binding agents of the invention may be chemically bonded with polymers, lipids, or other moieties.

The binding agents may comprise at least one of the CDRs described herein incorporated into a biocompatible framework structure. In one example, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions or substitutions of amino acids, relative to a naturally occurring polypeptide or fold. These scaffolds can be derived from a polypeptide of any species (or of more than one species), such as a human, other mammal, other vertebrate, invertebrate, plant, bacteria or virus.

Typically the biocompatible framework structures are based on protein scaffolds or skeletons other than immunoglobulin domains. For example, those based on fibronectin, ankyrin, lipocalin, neocarzinostain, cytochrome b, CP1 zinc finger, PST1, coiled coil, LAC1-D1, Z domain and tendramisat domains may be used (See e.g., Nygren and Uhlen, 1997, Current Opinion in Structural Biology, 7, 463-469).

In preferred embodiments, it will be appreciated that the binding agents of the invention include the humanized antibodies described herein. Humanized antibodies such as those described herein can be produced using techniques known to those skilled in the art (Zhang, W., et al., Molecular Immunology. 42(12): 1445-1451, 2005; Hwang W. et al., Methods. 36(1):35-42, 2005; Dall'Acqua W F, et al., Methods 36(1):43-60, 2005; and Clark, M., Immunology Today. 21(8):397-402, 2000).

Additionally, one skilled in the art will recognize that suitable binding agents include portions of these antibodies, such as one or more of CDR-H1, CDR-H2, CDR-H3, CDR-L1, CDR-L2 and CDR-L3 as specifically disclosed herein. At least one of the regions of CDR-H1, CDR-H2, CDR-H3, CDR-L1, CDR-L2 and CDR-L3 may have at least one amino acid substitution, provided that the binding agent retains the binding specificity of the non-substituted CDR. The non-CDR portion of the binding agent may be a non-protein molecule, wherein the binding agent cross-blocks the binding of an antibody disclosed herein to WISE and/or neutralizes WISE. The non-CDR portion of the binding agent may be a non-protein molecule in which the binding agent exhibits a similar binding pattern to human WISE peptides in a “human WISE peptide epitope competition binding assay” as that exhibited by at least one of antibodies described herein and/or neutralizes WISE. The non-CDR portion of the binding agent may be composed of amino acids, wherein the binding agent is a recombinant binding protein or a synthetic peptide, and the recombinant binding protein cross-blocks the binding of an antibody disclosed herein to WISE and/or neutralizes WISE. The non-CDR portion of the binding agent may be composed of amino acids, wherein the binding agent is a recombinant binding protein, and the recombinant binding protein exhibits a similar binding pattern to human WISE peptides in the human WISE peptide epitope competition binding assay (described hereinbelow) as that exhibited by at least one of the antibodies described herein Ab-AA, Ab-AB, Ab-AC, Ab-AD, Ab-AE, Ab-AF, Ab-AG, Ab-AH, Ab-AI, Ab-AJ D14 and antibodies humanized with the CDRs of Ab-AA, Ab-AB, Ab-AC, Ab-AD, Ab-AE, Ab-AF, Ab-AG, Ab-AH, Ab-AI, Ab-AJ and/or neutralizes WISE.

Where an antibody comprises one or more of CDR-H1, CDR-H2, CDR-H3, CDR-L1, CDR-L2 and CDR-L3 as described above, it may be obtained by expression from a host cell containing DNA coding for these sequences. A DNA coding for each CDR sequence may be determined on the basis of the amino acid sequence of the CDR and synthesized together with any desired antibody variable region framework and constant region DNA sequences using oligonucleotide synthesis techniques, site-directed mutagenesis and polymerase chain reaction (PCR) techniques as appropriate. DNA coding for variable region frameworks and constant regions is widely available to those skilled in the art from genetic sequences databases such as GenBank®. Each of the above-mentioned CDRs will be typically located in a variable region framework at positions 31-35 (CDR-H1), 50-65 (CDR-H2) and 95-102 (CDR-H3) of the heavy chain and positions 24-34 (CDR-L1), 50-56 (CDR-L2) and 89-97 (CDR-L3) of the light chain according to the Kabat numbering system (Kabat et al., 1987 in Sequences of proteins of Immunological Interest, U.S. Department of Health and Human Services, NIH, USA).

The term “CDR” can refer to the complementarity determining region within antibody variable sequences. There are typically three CDRs in each of the variable regions of the heavy chain and the light chain, which are designated CDR1, CDR2 and CDR3, for each of the variable regions. The boundaries of these CDRs have been defined differently according to different systems. The system described by Kabat (Kabat et al., Sequences of Proteins of Immunological Interest (National Institutes of Health, Bethesda, Md. (1987) and (1991)) provide a residue numbering system applicable to any variable region of an antibody, and provides residue boundaries defining the three CDRs. Chothia and coworkers (Chothia &Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may have boundaries that overlap with CDRs defined using Kabat. Other boundaries defining CDRs overlapping with the CDRs from Kabat have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat numbering, although they may be shortened or lengthened in light of prediction or experimental findings that particular residues or groups of residues or even entire CDRs do not significantly impact antigen binding. The methods used herein may utilize CDRs defined according to any of these systems.

As used herein, the term “framework” or “framework sequence” refers to the remaining sequences of a variable region around but not including the CDRs. Because the exact definition of a CDR sequence can be determined by different systems, the meaning of a framework sequence is subject to correspondingly different interpretations. The CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

Once synthesized, the DNA encoding an antibody of the invention or fragment thereof may be propagated and expressed according to any of a variety of well-known procedures for nucleic acid excision, ligation, transformation, and transfection using any number of known expression vectors. Thus, in certain embodiments expression of an antibody fragment may be preferred in a prokaryotic host, such as Escherichia coli (see, e.g., Pluckthun et al., 1989 Methods Enzymol. 178:497-515). In certain other embodiments, expression of the antibody or a fragment thereof may be preferred in a eukaryotic host cell, including yeast (e.g., Saccharomyces cerevisiae, Schizosaccharomyces pombe, and Pichia pastoris), animal cells (including mammalian cells) or plant cells. Examples of suitable animal cells include, but are not limited to, myeloma (such as a mouse NSO line), COS, CHO, or hybridoma cells. Examples of plant cells include tobacco, corn, soybean, and rice cells.

One or more replicable expression vectors containing DNA encoding an antibody variable and/or constant region may be prepared and used to transform an appropriate cell line, for example, a non-producing myeloma cell line, such as a mouse NSO line or a bacteria, such as E. coli, in which production of the antibody will occur. In order to obtain efficient transcription and translation, the DNA sequence in each vector should include appropriate regulatory sequences, particularly a promoter and leader sequence operatively linked to the variable domain sequence. Particular methods for producing antibodies in this way are generally well-known and routinely used. For example, basic molecular biology procedures are described by Maniatis et al. (Molecular Cloning, A Laboratory Manual, 2nd ed., Cold Spring Harbor Laboratory, New York, 1989; see also Maniatis et al, 3rd ed., Cold Spring Harbor Laboratory, New York, (2001)). DNA sequencing can be performed as described in Sanger et al. (PNAS 74:5463, (1977)) and the Amersham International plc sequencing handbook, and site directed mutagenesis can be carried out according to methods known in the art (Kramer et al., Nucleic Acids Res. 12:9441, (1984); Kunkel Proc. Natl. Acad. Sci. USA 82:488-92 (1985); Kunkel et al., Methods in Enzymol. 154:367-82 (1987); the Anglian Biotechnology Ltd handbook). Additionally, numerous publications describe techniques suitable for the preparation of antibodies by manipulation of DNA, creation of expression vectors, and transformation and culture of appropriate cells (Mountain A and Adair, J R in Biotechnology and Genetic Engineering Reviews (ed. Tombs, M P, 10, Chapter 1, 1992, Intercept, Andover, UK); “Current Protocols in Molecular Biology”, 1999, F. M. Ausubel (ed.), Wiley Interscience, New York).

Where it is desired to improve the affinity of antibodies according to the invention containing one or more of the above-mentioned CDRs can be obtained by a number of affinity maturation protocols including maintaining the CDRs (Yang et al., J. Mol. Biol., 254, 392-403, 1995), chain shuffling (Marks et al., Bio/Technology, 10, 779-783, 1992), use of mutation strains of E. coli. (Low et al., J. Mol. Biol., 250, 350-368, 1996), DNA shuffling (Patten et al., Curr. Opin. Biotechnol., 8, 724-733, 1997), phage display (Thompson et al., J. Mol. Biol., 256, 7-88, 1996) and sexual PCR (Crameri, et al., Nature, 391, 288-291, 1998). All of these methods of affinity maturation are discussed by Vaughan et al. (Nature Biotechnology, 16, 535-539, 1998).

Other antibodies according to the invention may be obtained by conventional immunization and cell fusion procedures as described herein and known in the art. Monoclonal antibodies of the invention may be generated using a variety of known techniques. In general, monoclonal antibodies that bind to specific antigens may be obtained by methods known to those skilled in the art (see, for example, Kohler et al., Nature 256:495, 1975; Coligan et al. (eds.), Current Protocols in Immunology, 1:2.5.12.6.7 (John Wiley & Sons 1991); U.S. Pat. Nos. RE 32,011, 4,902,614, 4,543,439, and 4,411,993; Monoclonal Antibodies, Hybridomas: A New Dimension in Biological Analyses, Plenum Press, Kennett, McKearn, and Bechtol (eds.) (1980); and Antibodies: A Laboratory Manual, Harlow and Lane (eds.), Cold Spring Harbor Laboratory Press (1988); Picksley et al., “Production of monoclonal antibodies against proteins expressed in E. coli,” in DNA Cloning 2: Expression Systems, 2nd Edition, Glover et al. (eds.), page 93 (Oxford University Press 1995)). Antibody fragments may be derived therefrom using any suitable standard technique such as proteolytic digestion, or optionally, by proteolytic digestion (for example, using papain or pepsin) followed by mild reduction of disulfide bonds and alkylation. Alternatively, such fragments may also be generated by recombinant genetic engineering techniques as described herein.

Monoclonal antibodies can be obtained by injecting an animal, for example, a rat, hamster, a rabbit, or preferably a mouse, including for example a transgenic or a knock-out, as known in the art, with an immunogen comprising WISE, for example, including both full length or the mature polypeptides depicted in SEQ ID NO: 2, 4, 6, or 8, or a fragment thereof, e.g., Seq Id No. 9, according to methods known in the art and described herein. The presence of specific antibody production may be monitored after the initial injection and/or after a booster injection by obtaining a serum sample and detecting the presence of an antibody that binds to human WISE or peptide using any one of several immunodetection methods known in the art and described herein. From animals producing the desired antibodies, lymphoid cells, most commonly cells from the spleen or lymph node, are removed to obtain B-lymphocytes. The B lymphocytes are then fused with a drug-sensitized myeloma cell fusion partner, preferably one that is syngeneic with the immunized animal and that optionally has other desirable properties (e.g., inability to express endogenous Ig gene products, e.g., P3X63-Ag 8.653 (ATCC No. CRL 1580); NSO, SP20) to produce hybridomas, which are immortal eukaryotic cell lines. The lymphoid (e.g., spleen) cells and the myeloma cells may be combined for a few minutes with a membrane fusion-promoting agent, such as polyethylene glycol or a nonionic detergent, and then plated at low density on a selective medium that supports the growth of hybridoma cells but not unfused myeloma cells. A preferred selection media is HAT (hypoxanthine, aminopterin, thymidine). After a sufficient time, usually about one to two weeks, colonies of cells are observed. Single colonies are isolated, and antibodies produced by the cells may be tested for binding activity to human WISE, using any one of a variety of immunoassays known in the art and described herein. The hybridomas are cloned (e.g., by limited dilution cloning or by soft agar plaque isolation) and positive clones that produce an antibody specific to WISE are selected and cultured. The monoclonal antibodies from the hybridoma cultures may be isolated from the supernatants of hybridoma cultures. An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Monoclonal antibodies can be isolated and purified by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)). Monoclonal antibodies may be purified by affinity chromatography using an appropriate ligand selected based on particular properties of the antibody (e.g., heavy or light chain isotype, binding specificity, etc.). Examples of a suitable ligand, immobilized on a solid support, include Protein A, Protein G, an anticonstant region (light chain or heavy chain) antibody, an anti-idiotype antibody, and a TGF-beta binding protein, or fragment or variant thereof.

An antibody of the present invention may also be a human monoclonal antibody. Human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein. For example, human monoclonal antibodies may be obtained from transgenic mice that have been engineered to produce specific human antibodies in response to antigenic challenge. Methods for obtaining human antibodies from transgenic mice are described, for example, by Green et al., Nature Genet. 7:13, 1994; Lonberg et al., Nature 368:856, 1994; Taylor et al., Int. Immun. 6:579, 1994; U.S. Pat. No. 5,877,397; Bruggemann et al., 1997 Curr. Opin. Biotechnol. 8:455-58; Jakobovits et al., 1995 Ann. N.Y. Acad. Sci. 764:525-35. In this technique, elements of the human heavy and light chain locus are introduced into strains of mice derived from embryonic stem cell lines that contain targeted disruptions of the endogenous heavy chain and light chain loci (see also Bruggemann et al., Curr. Opin. Biotechnol. 8:455-58 (1997)). For example, human immunoglobulin transgenes may be mini-gene constructs, or transloci on yeast artificial chromosomes, which undergo B cell-specific DNA rearrangement and hypermutation in the mouse lymphoid tissue. Human monoclonal antibodies may be obtained by immunizing the transgenic mice, which may then produce human antibodies specific for WISE. Lymphoid cells of the immunized transgenic mice can be used to produce human antibody-secreting hybridomas according to the methods described herein. Polyclonal sera containing human antibodies may also be obtained from the blood of the immunized animals.

Another method for generating human antibodies of the invention includes immortalizing human peripheral blood cells by EBV transformation. See, e.g., U.S. Pat. No. 4,464,456. Such an immortalized B cell line (or lymphoblastoid cell line) producing a monoclonal antibody that specifically binds to WISE can be identified by immunodetection methods as provided herein, for example, an ELISA, and then isolated by standard cloning-techniques. The stability of the lymphoblastoid cell line producing an anti-WISE antibody may be improved by fusing the transformed cell line with a murine myeloma to produce a mouse-human hybrid cell line according to methods known in the art (see, e.g., Glasky et al., Hybridoma 8:377-89 (1989)). Still another method to generate human monoclonal antibodies is in vitro immunization, which includes priming human splenic B cells with human WISE, followed by fusion of primed B cells with a heterohybrid fusion partner. See, e.g., Boerner et al., 1991 J. Immunol. 147:86-95.

In certain embodiments, a B cell that is producing an anti-human WISE antibody is selected and the light chain and heavy chain variable regions are cloned from the B cell according to molecular biology techniques known in the art (WO 92/02551; U.S. Pat. No. 5,627,052; Babcook et al., Proc. Natl. Acad. Sci. USA 93:7843-48 (1996)) and described herein. B cells from an immunized animal may be isolated from the spleen, lymph node, or peripheral blood sample by selecting a cell that is producing an antibody that specifically binds to WISE. B cells may also be isolated from humans, for example, from a peripheral blood sample. Methods for detecting single B cells that are producing an antibody with the desired specificity are well known in the art, for example, by plaque formation, fluorescence-activated cell sorting, in vitro stimulation followed by detection of specific antibody, and the like. Methods for selection of specific antibody-producing B cells include, for example, preparing a single cell suspension of B cells in soft agar that contains human WISE. Binding of the specific antibody produced by the B cell to the antigen results in the formation of a complex, which may be visible as an immunoprecipitate. After the B cells producing the desired antibody are selected, the specific antibody genes may be cloned by isolating and amplifying DNA or mRNA according to methods known in the art and described herein.

An additional method for obtaining antibodies of the invention is by phage display. See, e.g., Winter et al., 1994 Annu. Rev. Immunol. 12:433-55; Burton et al., 1994 Adv. Immunol. 57:191-280. Human or murine immunoglobulin variable region gene combinatorial libraries may be created in phage vectors that can be screened to select Ig fragments (Fab, Fv, sFv, or multimers thereof) that bind specifically to TGF-beta binding protein or variant or fragment thereof. See, e.g., U.S. Pat. No. 5,223,409; Huse et al., 1989 Science 246:1275-81; Sastry et al., Proc. Natl. Acad. Sci. USA 86:5728-32 (1989); Alting-Mees et al., Strategies in Molecular Biology 3:1-9 (1990); Kang et al., 1991 Proc. Natl. Acad. Sci. USA 88:4363-66; Hoogenboom et al., 1992 J. Molec. Biol. 227:381-388; Schlebusch et al., 1997 Hybridoma 16:47-52 and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

Heavy and light chain immunoglobulin cDNA expression libraries may also be prepared in lambda phage, for example, using lambda ImmunoZap™ (H) and lambda ImmunoZap™ (L) vectors (Stratagene, La Jolla, Calif.). Briefly, mRNA is isolated from a B cell population, and used to create heavy and light chain immunoglobulin cDNA expression libraries in the lambda ImmunoZap(H) and lambda ImmunoZap(L) vectors. These vectors may be screened individually or co-expressed to form Fab fragments or antibodies (see Huse et al., supra; see also Sastry et al., supra). Positive plaques may subsequently be converted to a non-lytic plasmid that allows high level expression of monoclonal antibody fragments from E. coli.

In one embodiment, in a hybridoma the variable regions of a gene expressing a monoclonal antibody of interest are amplified using nucleotide primers. These primers may be synthesized by one of ordinary skill in the art, or may be purchased from commercially available sources. (See, e.g., Stratagene (La Jolla, Calif.), which sells primers for mouse and human variable regions including, among others, primers for VHa, VHb, VHc, VHd, CHI, VL and CL regions.) These primers may be used to amplify heavy or light chain variable regions, which may then be inserted into vectors such as ImmunoZAP™ H or ImmunoZAP™ (Stratagene), respectively. These vectors may then be introduced into E. coli, yeast, or mammalian-based systems for expression. Large amounts of a single-chain protein containing a fusion of the VH and VL domains may be produced using these methods (see Bird et al., Science 242:423-426, 1988).

Once cells producing antibodies according to the invention have been obtained using any of the above-described immunization and other techniques, the specific antibody genes may be cloned by isolating and amplifying DNA or mRNA therefrom according to standard procedures as described herein. The antibodies produced therefrom may be sequenced and the CDRs identified and the DNA coding for the CDRs may be manipulated as described previously to generate other antibodies according to the invention.

Preferably the binding agents specifically bind to WISE. As with all binding agents and binding assays, one of skill in this art recognizes that the various moieties to which a binding agent should not detectably bind in order to be therapeutically effective and suitable would be exhaustive and impractical to list. Therefore, for a binding agent disclosed herein, the term “specifically binds” refers to the ability of a binding agent to bind to WISE, preferably human WISE, with greater affinity than it binds to an unrelated control protein. Preferably the control protein is hen egg white lysozyme. Preferably the binding agents bind to WISE with an affinity that is at least, 50, 100, 250, 500, 1000, or 10,000 times greater than the affinity for a control protein. A binding agent may have a binding affinity for human WISE of less than or equal to 1×10⁻⁷M, less than or equal to 1×10⁻⁸M, less than or equal to 1×10⁻⁹M, less than or equal to 1×10⁻¹⁰ M, less than or equal to 1×10⁻¹¹ M, or less than or equal to 1×10⁻¹² M.

Affinity may be determined by an affinity ELISA assay. In certain embodiments, affinity may be determined by a BIAcore assay. In certain embodiments, affinity may be determined by a kinetic method. In certain embodiments, affinity may be determined by an equilibrium/solution method. Such methods are described in further detail herein or known in the art.

WISE binding agents of the present invention preferably modulate WISE function in the cell-based assay described herein and/or the in vivo assay described herein and/or bind to one or more of the epitopes described herein and/or cross-block the binding of one of the antibodies described in this application and/or are cross-blocked from binding WISE by one of the antibodies described in this application. Accordingly such binding agents can be identified using the assays described herein.

In certain embodiments, binding agents are generated by first identifying antibodies that bind to one more of the epitopes provided herein and/or neutralize in the cell-based and/or in vivo assays described herein and/or cross-block the antibodies described in this application and/or are cross-blocked from binding WISE by one of the antibodies described in this application. The CDR regions from these antibodies are then used to insert into appropriate biocompatible frameworks to generate WISE binding agents. The non-CDR portion of the binding agent may be composed of amino acids, or may be a non-protein molecule. The assays described herein allow the characterization of binding agents. Preferably the binding agents of the present invention are antibodies as defined herein.

It will be understood by one skilled in the art that some proteins, such as antibodies, may undergo a variety of posttranslational modifications during expression and secretion from host cells. The type and extent of these modifications often depends on the host cell line used to express the protein as well as the culture conditions. Such modifications may include variations in glycosylation, methionine or tryptophan oxidation, diketopiperizine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995). Once the proteins have been expressed and processed they are in a ‘mature’ form. Thus it is understood that the invention includes mature antibodies that result from expression of the DNAs of the invention.

Antibodies disclosed herein bind to regions of human WISE which are important for the in vivo activity of the protein thereby inhibiting the activity of WISE. Binding of an antibody to WISE can be correlated with changes in biomarkers associated with kidney function, for example urinary levels of albumin or 24 hours total urinary protein excretion, serum creatinine or creatinine clearance rate. Methods of constructing and expressing antibodies and fragments thereof comprising CDR's of the present invention are known to those of skill in the art.

An oligopeptide or polypeptide is within the scope of the invention if it has an amino acid sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to at least one of the CDR's depicted in Table 1; and/or to a CDR of a WISE binding agent that cross-blocks the binding of at least one of antibodies described herein to loop 2 of WISE, and/or is cross-blocked from binding to WISE by at least one of antibodies described herein; and/or to a CDR of a WISE binding agent wherein the binding agent can block the inhibitory effect of WISE in a cell based assay or activate the effect of WISE in a cell based assay (i.e. a WISE neutralizing binding agent); and/or to a CDR of a WISE binding agent that binds to a cystine knot domain epitope.

WISE binding agent polypeptides and antibodies are within the scope of the invention if they have amino acid sequences that are at least 85%, 86%, 87%, 88%, 89%, 90%, 91%, 92%, 93%, 94%, 95%, 96%, 97%, 98% or 99% identical to a variable region of at least one of antibodies that bind to loop 2 of WISE (e.g., Seq Id No. 9), and cross-block the binding of at least one of antibodies that binds to loop 2 of WISE, and/or are cross-blocked from binding to WISE loop 2 by at least one of antibodies described herein; and/or can block the inhibitory effect of WISE in a cell based assay (i.e. a WISE neutralizing binding agent); and/or bind to a cystine knot domain epitope. The invention also contemplates an isolated antibody or fragment thereof wherein said WISE antibody or fragment thereof can decrease at least one of following parameters: tissue injury and markers thereof, Sirius red staining or collagen production, expression of myofibroblast markers such as aSMA or FSP-1, osteopontin expression, proteinuria, and/or can alter the activity of WISE in a cell based assay. As used herein, one of skill in the art will appreciate that alteration of activity includes activation or inhibition.

Polynucleotides encoding WISE binding agents are within the scope of the invention if they have polynucleotide sequences that are at least 85%, 86%, 87%, 88%, 89%, 90%, 91%, 92%, 93%, 94%, 95%, 96%, 97%, 98% or 99% identical to a polynucleotide encoding a variable region of at least one of antibodies Ab-AA, Ab-AB, and Ab-AC, and wherein the encoded WISE binding agents cross-block the binding of at least one of antibodies described herein; and/or can block the inhibitory effect of WISE in a cell based assay (i.e. a WISE neutralizing binding agent); and/or bind to a cystine knot domain epitope.

The affinity of a binding agent such as an antibody or binding partner, as well as the extent to which a binding agent (such as an antibody) inhibits binding, can be determined by one of ordinary skill in the art using conventional techniques, for example those described by Scatchard et al. (Ann. N.Y. Acad. Sci. 51:660-672 (1949)) or by surface plasmon resonance (SPR; BIAcore, Biosensor, Piscataway, N.J.). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al., Cancer Res. 53:2560-65 (1993)).

An antibody according to the present invention may belong to any immunoglobin class, for example IgG, IgE, IgM, IgD, or IgA. It may be obtained from or derived from an animal, for example, fowl (e.g., chicken) and mammals, which includes but is not limited to a mouse, rat, hamster, rabbit, or other rodent, cow, horse, sheep, goat, camel, human, or other primate. The antibody may be an internalizing antibody. Production of antibodies is disclosed generally in U.S. patent Publication No. 2004/0146888 A1.

### Characterization Assays

In the methods described herein to generate antibodies according to the invention, including the manipulation of the specific loop 2 WISE antibody CDRs into new frameworks and/or constant regions, appropriate assays are available to select the desired antibodies or binding agents (i.e. assays for determining binding affinity to WISE; cross-blocking assays; Biacore-based “human WISE peptide epitope competition binding assay;” MC3T3-E1 cell based assay; in vivo assays).

### Epitope Binding Assays

The unprocessed human WISE is 206 amino acids with the signal peptide and the mature form of human WISE is a 183 amino acid glycoprotein containing a cystine-knot motif. Due to conservation of key amino acid residues, particularly the cysteines, it is believed that WISE has a structure similar to previously described cystine knot proteins. This structure includes, in addition to the cystine-knot motif, three loops designated as Loop 1, Loop 2 and Loop 3. As used herein, the positions of the loops are defined as approximately at amino acids 75 to 104 of SEQ ID NO: 2 for Loop 1; Loop 2 is approximately at amino acids 105 to 132; and Loop 3 is approximately at amino acids 134 to 170 of SEQ ID NO:2. It is understood that approximate positions mean that the relative positions could be plus or minus 3 amino acids carboxy terminal or amino terminal of the stated positions.

Human WISE can be subjected to proteolytic digestion to produce fragments. Briefly, using different proteases, including trypsin, Asp-N, and Lys-C, fragments with various cleavage sites and sizes are generated. The sequences and mass for various human WISE peptides are determined. Antibody protection is evaluated to determine the effect on accessibility for proteolysis, including clipped site masking and peptide shifting. Finally, a BIAcore-based “human WISE peptide epitope competition assay” is performed.

One group of antibodies exhibits a specific pattern of binding to certain epitopes as evidenced by a Biacore-based “human WISE peptide epitope competition binding assay.” Briefly, the antibody is preincubated with the epitope to be tested, at concentrations that will saturate the epitope-binding sites on the antibody. The antibody is then exposed to WISE bound to a chip surface. After the appropriate incubation and washing procedures, a pattern of competitive binding is established.

### Cross-Blocking Assays

The terms “cross-block”, “cross-blocked” and “cross-blocking” are used interchangeably herein to mean the ability of an antibody or other binding agent to interfere with the binding of other antibodies or binding agents to WISE.

The extent to which an antibody or other binding agent is able to interfere with the binding of another to WISE, and therefore whether it can be said to cross-block according to the invention, can be determined using competition binding assays. One particularly suitable quantitative assay uses a Biacore machine which can measure the extent of interactions using surface plasmon resonance technology. Another suitable quantitative cross-blocking assay uses an ELISA-based approach to measure competition between antibodies or other binding agents in terms of their binding to WISE.

It is also contemplated that a type of cross blocking assay is suitable for use in identifying peptides that inhibit binding of WISE to its receptors LRP-5 and LRP-6. For example, a loop-2 peptide or a loop-2 peptide circularized by use of cysteine disulfide linkages have been shown to inhibit binding of WISE to its receptor.

One of skill in the art will appreciate that the terms associated with cross-blocking are not meant to be taken as absolute blocking, rather it is a term of art understood to mean that there is reduced binding due to steric or other interference from prior binding in a region that the test molecule also binds, thereby reducing the amount of binding of the test molecule. Thus it is appreciate that there can be cross-blocking when there is a detectable reduction in binding of a test antibody to a target. This detectable reduction in binding can be as little as 15%, 10%, or less depending on the sensitivity of the assay.

### Biacore Cross-Blocking Assay

The following generally describes a suitable Biacore assay for determining whether an antibody or other binding agent cross-blocks or is capable of cross-blocking according to the invention. For convenience reference is made to two antibodies, but it will be appreciated that the assay can be used with any of the WISE binding agents described herein. The Biacore machine (for example the Biacore 3000) is operated in line with the manufacturer's recommendations.

Thus in one cross-blocking assay, WISE is coupled to a CM5 Biacore chip using standard amine coupling chemistry to generate a WISE-coated surface. Typically 200-800 resonance units of WISE would be coupled to the chip (an amount that gives easily measurable levels of binding but that is readily saturable by the concentrations of test reagent being used).

The two antibodies (termed A* and B*) to be assessed for their ability to cross-block each other are mixed at a one to one molar ratio of binding sites in a suitable buffer to create the test mixture. When calculating the concentrations on a binding site basis the molecular weight of an antibody is assumed to be the total molecular weight of the antibody divided by the number of WISE binding sites on that antibody.

The concentration of each antibody in the test mix should be high enough to readily saturate the binding sites for that antibody on the WISE molecules captured on the Biacore chip. The antibodies in the mixture are at the same molar concentration (on a binding basis) and that concentration would typically be between 1.00 and 1.5 micromolar (on a binding site basis).

Separate solutions containing antibody A* alone and antibody B* alone are also prepared. Antibody A* and antibody B* in these solutions should be in the same buffer and at the same concentration as in the test mix.

The test mixture is passed over the WISE-coated Biacore chip and the total amount of binding recorded. The chip is then treated in such a way as to remove the bound antibodies without damaging the chip-bound WISE. Typically this is done by treating the chip with 30 mM HCl for 60 seconds.

The solution of antibody A* alone is then passed over the WISE-coated surface and the amount of binding recorded. The chip is again treated to remove all of the bound antibody without damaging the chip-bound WISE.

The solution of antibody B* alone is then passed over the WISE-coated surface and the amount of binding recorded.

The maximum theoretical binding of the mixture of antibody A* and antibody B* is next calculated, and is the sum of the binding of each antibody when passed over the WISE surface alone. If the actual recorded binding of the mixture is less than this theoretical maximum then the two antibodies are cross-blocking each other.

Thus, in general, a cross-blocking antibody or other binding agent according to the invention is one which will bind to WISE in the above Biacore cross-blocking assay such that during the assay and in the presence of a second antibody or other binding agent of the invention the recorded binding is between 80% and 0.1% (e.g. 80% to 4%) of the maximum theoretical binding, specifically between 75% and 0.1% (e.g. 75% to 4%) of the maximum theoretical binding, and more specifically between 70% and 0.1% (e.g. 70% to 4%) of maximum theoretical binding (as just defined above) of the two antibodies or binding agents in combination.

The Biacore assay described above is an assay used to determine if antibodies or other binding agents cross-block each other according to the invention. On rare occasions particular antibodies or other binding agents may not bind to WISE coupled via amine chemistry to a CM5 Biacore chip (this usually occurs when the relevant binding site on WISE is masked or destroyed by the coupling to the chip). In such cases cross-blocking can be determined using a tagged version of WISE, for example N-terminal His-tagged WISE. In this particular format, an anti-His antibody would be coupled to the Biacore chip and then the His-tagged WISE would be passed over the surface of the chip and captured by the anti-His antibody. The cross blocking analysis would be carried out essentially as described above, except that after each chip regeneration cycle, new His-tagged WISE would be loaded back onto the anti-His antibody coated surface. In addition to the example given using N-terminal His-tagged WISE, C-terminal His-tagged WISE could alternatively be used. Furthermore, various other tags and tag binding protein combinations that are known in the art could be used for such a cross-blocking analysis (e.g. HA tag with anti-HA antibodies; FLAG tag with anti-FLAG antibodies; biotin tag with streptavidin).

### Elisa-Based Cross-Blocking Assay

The following generally describes an ELISA assay for determining whether an anti-WISE antibody or other WISE binding agent cross-blocks or is capable of cross-blocking according to the invention. For convenience, reference is made to two antibodies, but it will be appreciated that the assay can be used with any of the WISE binding agents described herein.

The general principal of the assay is to have an anti-WISE antibody coated onto the wells of an ELISA plate. An excess amount of a second, potentially cross-blocking, anti-WISE antibody is added in solution (i.e. not bound to the ELISA plate). A limited amount of WISE is then added to the wells. The coated antibody and the antibody in solution compete for binding of the limited number of WISE molecules. The plate is washed to remove WISE that has not been bound by the coated antibody and to also remove the second, solution phase antibody as well as any complexes formed between the second, solution phase antibody and WISE. The amount of bound WISE is then measured using an appropriate WISE detection reagent. An antibody in solution that is able to cross-block the coated antibody will be able to cause a decrease in the number of WISE molecules that the coated antibody can bind relative to the number of WISE molecules that the coated antibody can bind in the absence of the second, solution phase, antibody.

This assay is described in more detail further below for Ab-AA, Ab-AC and Ab-AE. In the instance where Ab-AA is chosen to be the immobilized antibody, it is coated onto the wells of the ELISA plate, after which the plates are blocked with a suitable blocking solution to minimize non-specific binding of reagents that are subsequently added. An excess amount of Ab-AC is then added to the ELISA plate such that the moles of Ab-AC WISE binding sites per well are at least 10 fold higher than the moles of Ab-AA WISE binding sites that were used, per well, during the coating of the ELISA plate.

WISE is then added such that the moles of WISE added per well are at least 25-fold lower than the moles of Ab-AA WISE binding sites that were used for coating each well. Following a suitable incubation period the ELISA plate is washed and a WISE detection reagent is added to measure the amount of WISE specifically bound by the coated anti-WISE antibody (in this case Ab-AA). The background signal for the assay is defined as the signal obtained in wells with the coated antibody (in this case Ab-AA), second solution phase antibody (in this case Ab-AB), WISE buffer only (i.e. no WISE) and WISE detection reagents. The positive control signal for the assay is defined as the signal obtained in wells with the coated antibody (in this case Ab-AA), second solution phase antibody buffer only (i.e. no second solution phase antibody), WISE and WISE detection reagents. The ELISA assay needs to be run in such a manner so as to have the positive control signal at least 3 times the background signal.

To avoid any artifacts (e.g. significantly different affinities between Ab-AA and Ab-AB for WISE) resulting from the choice of which antibody to use as the coating antibody and which to use as the second (competitor) antibody, the cross-blocking assay needs to be run in two formats: 1) format 1 is where the first antibody is the antibody that is coated onto the ELISA plate and second antibody is the competitor antibody that is in solution and 2) format 2 is where the first and second antibody are reversed in coating and solution.

### Cell Based Neutralization Assay

MC3T3-E1 SuperTopFlash (STF) reporter cells are used to determine whether WISE protein can modulate Wnt signaling. The activation of TCF-dependent signaling in MC3T3-E1 STF cells can be triggered using either endogenous Wnt signaling induced by switching culturing medium to differentiation medium or by adding exogenous Wnt such as Wnt3a. Recombinant WISE protein derived from either E coli or mammalian cell can dose-dependently inhibit Wnt signaling in MC3T3-E1 STF cells.

Luciferase assay: a vial of MC3T3-E1/STF cells is plated into a culture flask in expansion medium. When the cells are confluent they are trypsinized and cells in expansion medium plated into each well in 96 well plate. Next day all expansion medium is removed and replaced with 100 ul of freshly prepared differentiation medium.

Half of the differentiation medium (50 ul) was replaced with freshly prepared differentiation medium every day for the next four days. After five days of differentiation, all medium is replaced with testing samples in the fresh differentiation medium in 100 ul volume. The plates are then allowed to incubate for 24 hours before luciferase signal are measured. Luciferase signal is measured upon removal of medium from testing plates and addition of 20 ul of 1× lysis buffer that has been equilibrated to room temperature. The plate is sealed and rocked for 30 minutes at room temperature and 100 ul of luciferase assay reagent was added to each well and the signal was captured using Luminometer (LMAX, Molecular Device) according to manufacturer's instruction.

### In Vivo Neutralization Assay

Increases in various parameters associated with, or that result from, renal protection or pulmonary protection can be measured as an output from in vivo testing of WISE binding agents in order to identify those binding agents that are able to neutralize WISE and provide a therapeutic benefit. Such parameters include various renal/pulmonary markers and histomorphometric markers of renal/pulmonary health. A WISE neutralizing binding agent is defined as one capable of causing a statistically significant increase, as compared to vehicle treated animals, in any parameter associated with, or that results from, the stimulation of renal/pulmonary protection. Such in vivo testing can be performed in any suitable mammal (e.g. mouse, rat, monkey).

### Formulation and Delivery of Therapeutics

Pharmaceutical compositions are provided, comprising one of the above-described binding agents such as at least one of antibody to human WISE described herein, along with a pharmaceutically or physiologically acceptable carrier, excipient, or diluent.

The development of suitable dosing and treatment regimens for using the particular compositions described herein in a variety of treatment regimens, including e.g., subcutaneous, oral, parenteral, intravenous, intranasal, and intramuscular administration and formulation, is well known in the art, some of which are briefly discussed below for general purposes of illustration.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain circumstances it will be desirable to deliver the pharmaceutical compositions disclosed herein subcutaneously, parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, Remington's Pharmaceutical Sciences, 15th ed., pp. 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol. 16(7):307-21, 1998; Takakura, Nippon Rinsho 56(3):691-95, 1998; Chandran et al., Indian J. Exp. Biol. 35(8):801-09, 1997; Margalit, Crit. Rev. Ther. Drug Carrier Syst. 12(2-3):233-61, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety). The use of liposomes does not appear to be associated with autoimmune responses or unacceptable toxicity after systemic delivery. In certain embodiments, liposomes are formed from phospholipids that are dispersed in an aqueous medium and spontaneously form multilamellar concentric bilayer vesicles (also termed multilamellar vesicles (MLVs)).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev. Ind. Pharm. 24(12):1113-28, 1998). To avoid side effects due to intracellular polymeric overloading, such ultrafine particles (sized around 0.1 um) may be designed using polymers able to be degraded in vivo. Such particles can be made as described, for example, by Couvreur et al., Crit. Rev. Ther. Drug Carrier Syst. 5(1):1-20, 1988; zur Muhlen et al., Eur. J. Pharm. Biopharm. 45(2):149-55, 1998; Zambaux et al., J Controlled Release 50(1-3):31-40, 1998; and U.S. Pat. No. 5,145,684.

In addition, pharmaceutical compositions of the present invention may be placed within containers, along with packaging material that provides instructions regarding the use of such pharmaceutical compositions. Generally, such instructions will include a tangible expression describing the reagent concentration, as well as within certain embodiments, relative amounts of excipient ingredients or diluents (e.g., water, saline or PBS) that may be necessary to reconstitute the pharmaceutical composition.

The dose administered may range from 0.01 mg/kg to 200 mg/kg of body weight. Typical dosages are between 30 mg/kg and 75 mg/kg. However, as will be evident to one of skill in the art, the amount and frequency of administration will depend, of course, on such factors as the nature and severity of the indication being treated, the desired response, the condition of the patient, and so forth. Typically, the compositions may be administered by a variety of techniques, as noted above.

### Method of Treatment Using WISE Binding Agents

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented.

“Mammal” for purposes of treatment refers to any animal classified as a mammal, including humans, domestic and farm animals, and zoo, sports, or pet animals, such as dogs, horses, cats, cows, etc. Preferably, the mammal is human.

As used in the context of treating renal disorders or diseases, the phrase “therapeutically effective amount” is meant to refer to an amount of therapeutic or prophylactic WISE antibody that provides a reduction in renal damage or deterioration, or that provides a reduction in the severity or progression of symptoms associated with renal disease, such as fibrosis and/or proteinuria (i.e. that provides “therapeutic efficacy”) or preserves or improves renal function as measured using serum creatinine or creatinine clearance rate. As used in the context of treating fibrosis the phrase “therapeutically effective amount” is meant to refer to an amount of therapeutic or prophylactic WISE antibody that provides a reduction in fibroid elements or their precursors, and/or that provides a reduction in the severity or progression of symptoms associated with fibrotic disease (i.e. that provides “therapeutic efficacy”), e.g., proteinuric glomerular disease.

In one embodiment, the compositions of the invention are contemplated to be useful for treating, reducing and/or preventing renal dysfunction including those selected from the group consisting of proteinuric glomerular disease, end stage renal disease, chronic renal disease such as diabetic nephropathy, transplant related graft dysfunction, IgA nephropathy, Bartter's syndrome, Gitelman syndrome, nephrolithiasis, renal amyloidosis, hypertension, primary aldosteronism, Addison's disease; renal failure; glomerulonephritis and chronic glomerulonephritis: tubulointerstitial nephritis; cystic disorders of the kidney and dysplastic malformations such as polycystic disease, renal dysplasias, and cortical or medullary cysts; inherited polycystic renal diseases (PRD), such as recessive and autosomal dominant PRD; medullary cystic disease; medullary sponge kidney and tubular dysplasia; Alport's syndrome; non-renal cancers which affect renal physiology, such as bronchogenic tumors of the lungs or tumors of the basal region of the brain; multiple myeloma; adenocarcinomas of the kidney; metastatic renal carcinoma; in addition, nephrotoxic disorders include any functional or morphologic change in the kidney produced by any pharmaceutical, chemical, or biological agent that is ingested, injected, inhaled, or absorbed. Some broad categories of common nephrotoxic agents include but are not limited to immune suppressants, such as calcineurin inhibitors, heavy metals, all classes of antibiotics, analgesics, solvents, oxalosis-inducing agents, anticancer drugs, herbicides and pesticides, botanicals and biologicals, and antiepileptics.

The phrase “fibrotic-reducing activity” is meant to refer to the ability to inhibit, fully or partially, fibroid formation or to remove or reduce existing fibrosis. Thus, in one embodiment the compositions of the present invention are contemplated to be useful for treat fibrotic diseases, including pathological fibrosis or scarring (including endocardial sclerosis), idiopathic interstitial fibrosis, interstitial pulmonary fibrosis, perimuscular fibrosis, Symmers' fibrosis, pericentral fibrosis, hepatitis, dermatofibroma, billary cirrhosis, alcoholic cirrhosis, acute pulmonary fibrosis, idiopathic pulmonary fibrosis, acute respiratory distress syndrome, kidney fibrosis/glomerulonephritis, kidney fibrosis/diabetic nephropathy, scleroderma/systemic, scleroderma/local, keloids, hypertrophic scars, severe joint adhesions/arthritis, myelofibrosis, corneal scarring, cystic fibrosis, muscular dystrophy (duchenne's), cardiac fibrosis, muscular fibrosis/retinal separation, esophageal stricture and payronles disease. Further fibrotic disorders may be induced or initiated by surgery, including scar revision/plastic surgeries, glaucoma, cataract fibrosis, corneal scarring, joint adhesions, graft vs. host disease (e.g., in transplant patients), tendon surgery, nerve entrapment, dupuytren's contracture, OB/GYN adhesions/fibrosis, pelvic adhesions, peridural fibrosis, restenosis. It is also contemplated that fibrotic conditions where deposition of multiple extracellular matrix proteins, including but not limited to collagen and/or fibronectin, is a causative factor can be treated according to the invention. Idiopathic pulmonary fibrosis, bleomycin lung, cystic fibrosis, and glomerular nephropathy, including disease characterized by, for example, collagen and/or fibronectin deposits in the kidneys ultimately leading to renal failure are examples of conditions which can also be treated in accordance with the present invention.

The invention also contemplates an antibody that has an affinity of at less than 1×10⁻⁷M to WISE and inhibits WISE activity for use in a method for treating a medical condition associated with fibrosis, wherein the fibrosis can be associated with a disease discussed above including lung disease or kidney disease. Furthermore, also contemplated is an antibody that has an affinity of at less than 1×10⁻⁷M to WISE and inhibits WISE activity suitable for use in a method for treating a medical condition associated with proteinuria.

The invention also provides for combination therapies where the compositions of the invention are administered to a patient in need thereof with additional therapeutic agents that either treat the underlying disease or reduce symptoms associated with the disease being treated. These additional therapies can be administered simultaneously, before or after the administration of the composition of the present invention. Additional therapies for use in combination with the compositions of the present invention include ACE inhibitors, angiotensin receptor blockade (ARB), erythropoietin (e.g., Aranesp® (darbepoetin), Epogen® (erythropoietin alfa), calcineurin inhibitors, steroids, beta blockers and the like.

The invention also provides a diagnostic kit comprising at least one anti-WISE binding agent according to the present invention. The binding agent may be an antibody. In addition, such a kit may optionally comprise one or more of the following: (1) instructions for using the one or more binding agent(s) for screening, diagnosis, prognosis, therapeutic monitoring or any combination of these applications; (2) a labeled binding partner to the anti-WISE binding agent(s); (3) a solid phase (such as a reagent strip) upon which the anti-WISE binding agent(s) is immobilized; and (4) a label or insert indicating regulatory approval for screening, diagnostic, prognostic or therapeutic use or any combination thereof. If no labeled binding partner to the binding agent(s) is provided, the binding agent(s) itself can be labeled with one or more of a detectable marker(s), e.g. a chemiluminescent, enzymatic, fluorescent, or radioactive moiety.

The following examples are offered by way of illustration, and not by way of limitation.

### EXAMPLES

### Construct Preparation for Wise and Wise Loop-2 Mutant

hWise was amplified by PCR primers from a DNA clone containing the cDNA for hWise (NM_{—}015464). The expression vector as well as the approximately 641 base pair PCR product were digested with XbaI and NotI restriction enzymes. The appropriate fragments were ligated to yield the hWise expression vector.

The following describes the replacement of loop 2 of human WISE with hSost loop 2. Cloning of hWise-hSost loop 2 chimeric protein: substitution mutation was generated by a combination of primer extension and overlap PCR. The loop2 chimeric mutant has 75 bp of loop2 of hWise substituted with 63 bp of loop2 of hSost. Specifically, using hWise as a template the N-terminus of hWise was amplified with primers and extended. The C-terminus of hWise was amplified with primers and extended. The N-terminal and C-terminal fragments were used as templates in an overlap PCR reaction. The PCR product was digested with XbaI and NotI restriction enzymes and subcloned into a mammalian expression vector.

### Expression and Purification of Mouse and Human WISE in Mammalian Cells

One vial of stock culture was inoculated into 10 ml culture medium in Shake Flask (125 ml, Plastic), the culture was continued for 2-3 days; then the culture was expanded from 10 mL into 100 mL shake flask and again from 100 ml into 500 ml volume culture. For transfection, cells were seeded into 1 liter culture medium and grown until appropriate cell density.

Transfection mix was prepared, the cells were transfected using standard techniques and 24 hours post-transfection a feed was added to the cells. The culture was then continued for another 48 hours and conditioned medium was harvested by spinning at 4000 rpm for 30 minutes and then filter through a 0.2 uM filter. A small sample (1 ml) was then taken for western blot analysis and the rest was frozen down for purification. The host cell culture fluid (CCF) was centrifuged to remove cell debris. The CCF supernatant was then filtered.

A Heparin column was loaded with protein then washed with PBS until the absorbance at 280 nm of the flow-through returned to baseline. WISE protein was then eluted from the column using a linear gradient from 150 mM to 2 M sodium chloride in PBS and fractions collected. The fractions were then assayed by Coomassie-stained SDS-PAGE to identify fractions containing a polypeptide that migrates at the predicted size of WISE. The appropriate fractions from the column were combined to make the Heparin pool.

The WISE protein eluted from the Heparin column was further purified by reversed phase chromatography. The Heparin pool was made 22% ethanol and adjusted to pH 5.0 with acetic acid. The pool was filtered. The filtered Heparin pool was then loaded onto an equilibrated column. After loading, the column was washed until the absorbance at 280 nm of the flow-through returned to baseline. The WISE protein was then eluted from the column.

Following purification, the WISE was formulated in PBS by dialysis. Following formulation the WISE was filtered through a sterile 0.2 μm filter and stored at 4° C. or frozen.

### Antigen Modification and Immunizations

Human WISE protein from mammalian sources was emulsified in a 1:1 ratio using either Complete Freund's Adjuvant (Pierce) or RIBI (Sigma) then immunized sub-cutaneously and intraperitoneally into WISE knock out mice. Immunization occurred at least every 2 weeks and antiserum from the mice was taken after the 3rd immunizations for anti-WISE titer analysis.

### Fusions

Four days prior to fusion, each mouse was boosted intraperitoneally with Hu WISE protein in PBS. On the fusion day, the spleens were removed aseptically and the organs processed into a single cell suspension. The red blood cells were lysed and the spleenocytes were washed with RPMI (Gibco). Viable, log-phase growth myeloma cells were mixed with the murine spleenocytes in a 1:2.5 ratio of myeloma:spleenocytes. The cells were then washed 2 times in Cytofusion Medium C (Cytopulse Sciences Inc). After washing, the cells were suspended in 33% Cytofusion Medium C and 67% in-house derived hypo-osmolar fusion buffer at a final density of 1e7 cells/ml. This mixture was loaded into 2 ml BTX fusion chambers (Harvard Apparatus) then subjected to electrofusion conditions from a BTX ECM 2001 (Harvard Apparatus).

The cell suspensions were removed from the chambers and suspended in cell growth media. 20 μl per well of this cell suspension was plated into 384 well cell culture plates (Greiner) and incubated overnight in a 37° C. humidified 10% CO2 incubator. The following day, 20 μl of the above mentioned growth media containing 2×HAT (Sigma) was added to each well of the plates. The cultures were incubated for 7 days then the growth media was aspirated out of the wells and exchanged for fresh growth media. Screening of hybridoma supernatants commenced 2-3 days after the media change.

### Screening

High-binding clear polystyrene 384 well plates (Corning) were coated with 25 μg/well of a 1 ug/ml solution consisting of goat anti-mu IgG, Fc specific pAb (Pierce) in PBS. The plates were incubated with coating solution overnight 4° C. then washed once on an automatic plate washer using PBS+0.05% Tween 20 (Sigma). 50 μl of block solution was added to each well and incubated overnight at 4° C.

Five μl of hybridoma supernatant was transferred to each well of the ELISA plate and allowed to incubate for 60 min at room temp. The plates were then washed 2 times using the method described above. 20 μl/well of a 10 ng/ml solution of human WISE protein diluted in blocking solution was then added to each well of the plate. After the addition of the WISE antigen, the ELISA plates were allowed to incubate for 60 min at RT then washed. Next, 20 ul per well of a solution of rabbit-anti-WISE-HRP Pab (Amgen) diluted in blocking solution was added to each well and incubated for 60 min and plates were washed 4×.

Finally, 20 μl/well of TMB (Pierce) was added to each well and the plates were read on a Spectramax Plate Reader (Molecular Devices) at 650 nM. Cells from the ELISA positive hybridoma wells were subsequently expanded in cell culture for further characterization studies.

### High-Throughput Purification of IgGs

ELISA positive hybridoma clones identified during primary screen were transferred to 96-well plates using automated liquid transfer platform (Bravo) and allowed to grow for 3-5 days in a 37° C. humidified 5% CO2 incubator. Once the adequate cell mass was reached, each plate was duplicated into 8 plates (96-well) by transferring 20 ul of supernatant from original plate. Final volume of each plate was 200 ul. Plates were incubated for 7 days in a 37° C. humidified 5% CO2 incubator. Hybridoma supernatants were then collected into polypropylene assay block (2 ml, Costar 3961) and centrifuged at 3500 G for 30 minutes. Cleared supernatant devoid of cell debris were transferred to new assay blocks and incubated with 70 ul per well of Protein G Plus-Agarose (Calbiochem, Cat #IP08) overnight in room temperature on the shaker. An automated liquid handling robot utilizing custom software and a vacuum manifold system was then used to isolate and clarify the Protein G resin containing the bound IgG. The bound IgG was then eluted using standard low pH elution and neutralization conditions. The resulting purified IgG was quantitated via A280 absorbance.

### Binding Analysis to Identify Loop-2 Binders

Anti-WISE hybridoma supernatants were collected and added onto high-binding ELISA plates pre-coated with 1 ug/ml of goat anti-mouse IgG Fc (Pierce). Plates were incubated for 1 hour in room temperature. Plates were then washed 4× with Wash Buffer. Subsequently, huWISE or human WISE-hSost-loop2 chimeric protein at a final concentration of 2 ng/ml was added and incubated for 1 hour in room temperature. After 4× wash, rabbit anti-WISE Pab-biotin (50 ng/ml)+NeutrAvidin-HRP (Pierce) were added onto the plates and incubated for 1 hour in room temperature. Plates were again washed 4× with Wash Buffer. Binding was analyzed using 1-Step Ultra TMB-ELISA substrate (Pierce) according to the manufacturer's instructions.

### Identification of Neutralizing Antibodies in MC3T3-E1/STF-Luc Cell-Based Assay

MC3T3-E1 SuperTopFlash (STF) reporter cells are used to determine whether WISE protein can modulate Wnt signaling. The activation of TCF-dependent signaling in MC3T3-E1/STF-luc cells can be triggered using either endogenous Wnt signaling induced by switching culturing medium to differentiation medium or by adding exogenous Wnt such as Wnt3a protein. Recombinant WISE protein can dose-dependently inhibit Wnt signaling in MC3T3-E1/STF-luc cells.

A vial of MC3T3-E1/STF-luc cells is plated into a culture flask in expansion medium. When the cells reach to 90-95% confluent, they are trypsinized and cells in expansion medium plated 10K per well into 96 well test plate. Next day all expansion medium is removed and replaced with 100 ul of freshly prepared differentiation medium. 50% of the differentiation medium was replaced with freshly prepared differentiation medium every day for the next four days. After five days of differentiation, all medium is replaced with testing samples in the fresh differentiation medium in 100 ul volume. Pre-incubate WISE protein and WISE mab for 1 hour at 37 C before adding to the test well. The plates are then allowed to incubate for 24 hours before luciferase signal are measured. Luciferase signal is measured by using Promega's luciferase Assay System. Carefully removal of medium from testing plates, rinse cells with PBS and addition of 20 ul of 1× lysis buffer that has been equilibrated to room temperature. The plate is sealed and rocked for 30 minutes at room temperature and 100 ul of luciferase assay substrate was added to each well and the signal was captured using Luminometer (LMAX) according to manufacturer's instruction.

### Expression and Purification of Loop-2 Hybridoma CM

Single cells from the ELISA positive hybridoma wells were isolated using FACS sorting and placed into 96-well plates with 80 ul of BDR medium [50 ml Hybridoma Cloning Factor (BioVeris), 1×OPI medium supplement (Sigma), 55 uM 2-ME (Gibco), 10% Low IgG FBS (Gibco), 1×PSG (Gibco), BD Quantum Yield Medium (BD Bioscience)] per well. These cells were allowed to grow until the adequate cell mass was reached. Cells are then expanded further into 24-well plates with 1 ml of BDR medium per well. Once 24 well plates were confluent, cells were transferred to 6-well plates with 5 ml of BDR medium per well. After 5 days of incubation, half of the cells were frozen down in FBS (Ultra low IgG)+10% DMSO mix for backup. The other half was transferred into T-175 flask with 40 ml of BDR medium and allowed to expand. Once the T-175 flasks were confluent, supernatants were collected, filtered (0.45 um CA filter) and for purified.

Purification of WISE mAbs from Hybridoma Cell Culture for In Vitro Studies

WISE monoclonal antibodies (mAbs) were purified from hybridoma cell culture as follows. All purification processes were carried out at room temperature or 4° C. One purification scheme was used to purify the various mAbs and used affinity chromatography.

### Protein G Chromatography

The host cell culture fluid (CCF) was centrifuged in a Beckman Coulter Allegra X-12R centrifuge at 1500 rpm for 5 minutes at 10° C. to remove cell debris. The CCF supernatant was then filtered through a sterile 0.45 μm filter. At this point the sterile filtered CCF may be stored frozen until purification. If frozen the CCF was thawed at 40° C. Following thawing the CCF was filtered through a sterile 0.45 μm filter and then loaded onto Protein G chromatography media in the form of a column, Protein G High Performance (GE Healthcare, formerly Amersham Biosciences), equilibrated in PBS at room temperature.

After loading the Protein G column was washed with PBS until the absorbance at 280 nm of the flow-through returned to baseline. The WISE mAb was then eluted from the column using 0.1 M Acetate, pH 3 and immediately neutralized by adding 65 μl of a stock solution of 1 M Tris Base per mL of elution volume. The absorbance at 280 nm of the eluate was monitored and fractions containing protein were collected to make the Protein G pool.

### Formulation and Concentration

Formulation and concentration steps were performed at 4° C. Following purification the WISE mAbs were formulated in A5Su (10 mM sodium acetate, 9% sucrose, pH 5) by dialysis using 10,000 MWCO membranes (Pierce Slide-A-Lyzer). If concentration of WISE mAbs was necessary, a centrifugal device (Vivascience Vivaspin) with a 10,000 MWCO membrane was used. Alternatively, the Protein G pool can be buffer exchanged into A5Su and concentrated using the centrifugal device alone. Following formulation the WISE mAbs were filtered through a sterile 0.2 μm filter and stored at 40° C. or frozen.

### WISE Binds to LRP6 and the Binding can be Blocked by Neutralizing Anti-Wise Loop2 Antibody

The binding of WISE to putative receptor LRP6 was characterized using AlphaScreen technology. The following describes the detailed procedures of the assay:

1× buffer was prepared freshly daily follow the instruction provided with the AlphaScreen Histidine (Nickel Chelate) Detection Kit (PerkinElmer).

Working solution of biotinylated huWise, biotinylated huWise-huSost-loop2, rmLRP6-His6 (R&D Systems), anti-Wise loop2 antibodies were diluted with 1× buffer to 3 times of the final concentration desired for each protein and antibody reaction.

Five microliters each of Wise or Wise-huSost-loop2 and LRP6 working solution were dispensed into proper wells in a white opaque OptiPlate-384 microplate (PerkinElmer). After Wise and LRP6 reacting at R.T. for 1 hr with gentle shaking, 5 ul of each of the series diluted antibody working solution was added to those proper wells, let the reaction continue. The biotinylated-His6 (positive control, provided with Kit) series dilution and the nickel chelate acceptor and streptavidin donor beads (provided with Kit) working solution were prepared according to the same instruction. After antibody competing with LRP6 for Wise-binding for 60 min, 15 ul of each serially diluted Biotinylated-His6 solution was dispensed into the empty wells in the plate. Finally dispensed 5 ul of each donor and acceptor beads working solution into all sample and control wells, incubate in the dark at room temperature for one more hour and analyzed on EnVision microplate analyzer.

Construction of hWise Loop2 Ala Mutants

Alanine-scanning mutagenesis has been successful in systematically mapping functional binding epitopes. The full length form of WISE is 206 amino acids and the mature form of hWISE is a 183 amino acid glycoprotein containing a cystine-knot motif and three loops designated as Loop1, Loop2 and Loop-3. Loop 1 is at about amino acids and loop 3 is at Loop2 is approximately at amino acids from 105 to 132 of the full length human WISE and amino acids 82 to 109 of the mature form of human WISE. A total of 23 amino acid residues identified from position 107 to 129 in the hWISE Loop2 were changed to alanines.

Alanine scanning hWISE Loop2 genes were generated by site-directed mutagenesis using oligodeoxynucleotide primers of 24-30 nucleotides in length and wild type hWISE plasmid DNA as template. Reactions were performed as described in the QuikChange Site-Directed Mutagenesis Kit (Stragagene). Each mutant was created directly in a mammalian expression vector. Alanine mutant constructs were sequence confirmed and transfected into mammalian host cells for transient production of mutant proteins. Single amino acid mutation has no effect on protein expression.

Representative alanine substitutions are listed below with the amino acid change:

Leucine to alanine at amino acid 107 of Seq Id No.: 2.

Proline to alanine at amino acid 108 of Seq Id No.: 2.

Valine to alanine at amino acid 109 of Seq Id No.: 2.

Leucine to alanine at amino acid 110 of Seq Id No.: 2.

Proline to alanine at amino acid 111 of Seq Id No.: 2.

Asparagine to alanine at amino acid 112 of Seq Id No.: 2.

Tryptophan to alanine at amino acid 113 of Seq Id No.: 2.

Isoleucine to alanine at amino acid 114 of Seq Id No.: 2.

Glycine to alanine at amino acid 115 of Seq Id No.: 2.

Glycine to alanine at amino acid 116 of Seq Id No.: 2.

Glycine to alanine at amino acid 117 of Seq Id No.: 2.

Tyrosine to alanine at amino acid 118 of Seq Id No.: 2.

Glycine to alanine at amino acid 119 of Seq Id No.: 2.

Threonine to alanine at amino acid 120 of Seq Id No.: 2.

Lysine to alanine at amino acid 121 of Seq Id No.: 2.

Tyrosine to alanine at amino acid 122 of Seq Id No.: 2.

Tryptophan to alanine at amino acid 123 of Seq Id No.: 2.

Serine to alanine at amino acid 124 of Seq Id No.: 2.

Arginine to alanine at amino acid 125 of Seq Id No.: 2.

Arginine to alanine at amino acid 126 of Seq Id No.: 2.

Serine to alanine at amino acid 127 of Seq Id No.: 2.

Serine to alanine at amino acid 128 of Seq Id No.: 2.

Glutamine to alanine at amino acid 128 of Seq Id No.: 2.

### Epitope Mapping of Loop2 Binders and Residues Critical for Binding

Alanine-scanning mutagenesis was performed on hWISE Loop2 region and total 23 mutants were generated and assayed in vitro for antibody binding and functional characteristics.

Relative capture of individual WISE mutant proteins or wild type proteins by either neutralizing antibodies or non-neutralizing antibodies was compared to assess whether any of these single amino acid changes affects the binding of an antibody to WISE protein. The bound WISE proteins were then detected using HRP-conjugated affinity-purified polyclonal antibody against WISE.

Purified anti-WISE antibodies (0.5 ug/ml) were added onto high-binding ELISA plates pre-coated with 1 ug/ml of goat anti-mouse IgG Fc (Pierce). Plates were incubated for 1 hour in room temperature. Plates were then washed 4× with Wash Buffer. Subsequently alanine scanned WISE loop-2 mutant supernatants from transient transfection cultures (100× diluted) were added and incubated for 1 hour in room temperature. After 4× wash, rabbit anti-WISE Pab-biotin (50 ng/ml)+NeutrAvidin-HRP (Pierce, Cat #31001) were added onto the plates and incubated for 1 hour in room temperature. Plates were again washed 4× with Wash Buffer. Binding was analyzed using 1-Step Ultra TMB-ELISA substrate (Pierce; Cat #34028) according to the manufacturer's instructions.

A panel of loop 2 binding antibodies was applied to the alanine mutants and certain amino acids were identified as important to binding by these antibodies. These amino acids include one or more amino acids selected from the a group consisting of an asparagine at amino acid residue 112 of SEQ ID NO: 2, an isoleucine at amino acid residue 114 of SEQ ID NO: 2, a glycine at amino acid residue 115 of SEQ ID NO: 2, a glycine at amino acid residue 117 of SEQ ID NO: 2, a glycine at amino acid residue 119 of SEQ ID NO: 2, a lysine at amino acid residue 121 of SEQ ID NO: 2, a tryptophan at amino acid residue 123 of SEQ ID NO: 2, an arginine at amino acid residue 126 of SEQ ID NO: 2, or a glutamine at amino acid residue 129 of SEQ ID NO: 2.

These data provided examples of mutating with only one of the above mentioned residues significantly reduced the binding of the WISE antibodies to WISE protein. It is conceivable that a combination of the above mentioned residue may be important for optimal binding of certain loop-2 binding antibody. However a single residue could be enough to confer the binding of an antibody to the loop-2 of WISE.

### Cloning of the Murine Anti-huWISE Antibody Heavy and Light Chains

The sequences of the murine anti-human WISE light chain and heavy chain variable regions were obtained by the polymerase chain reaction (PCR) amplification technique known as 5′ RACE (rapid amplification of cDNA ends)., Total RNA was isolated from six murine hybridomas expressing human WISE binding monoclonal antibodies, Ab-AA, Ab-AB, Ab-AC, Ab-AD, Ab-AE, and Ab-AF, using TRIzol reagent (Invitrogen) followed by a further purification using the RNeasy Mini Kit (Qiagen). Oligo-dT primed first strand, RACE ready cDNAs were prepared using the GeneRacer Kit (Invitrogen). PCR amplifications of the cDNAs were performed with Phusion HF DNA polymerase (Finnzymes). The RACE PCR products were cloned into pCR4-TOPO (Invitrogen) and their sequences determined using ABI DNA sequencing instruments (Perkin Elmer). Consensus sequences were determined using Vector NTI Advance 10 software (Invitrogen).

### Humanization of Murine Human Anti-WISE Antibodies

Ab-AB was humanized using the light chain v1 with straight CDR graft into VK1|O018 acceptor framework. Ab Ab-AB was humanized using the heavy chain v1 with CDR graft with murine residues at A24T, R71A and A93T into VH1|1-46 acceptor framework. Ab-AB was also humanized using the light chain v2, with F71Y and Y87F back-mutations. Ab-AB was humanized using the heavy chain v2 with v1 with additional V2I, VTM 67, 68, 69 to AKL and V78A (Seq Id Nos.: 114 and 116 and Seq Id Nos.: 118 and 120 respectively).

Ab-AI was humanized using the light chain v1 with straight CDR graft into VK4|B3 acceptor framework. Ab-AI was humanized using the heavy chain v1 with S30T back-mutation. Back mutations were also introduced as Ab-AI was also humanized using the light chain v2 with Y49S back mutation (Kabat). Ab-AI was humanized using the heavy chain v2 with straight CDR graft into VH2|2-70 acceptor framework (Seq Id Nos.: 122 and 124 and Seq Id Nos.: 126 and 128).

Ab-AJ was humanized using the light chain with straight CDR graft into VK4|B3 acceptor framework. Ab-AJ was humanized using heavy chain CDR graft into VH1|1-46 acceptor framework with murine residues kept in place at Kabat positions 27, 28, 29, 30, 71, 93, 94 (Y27F, T28N, F29I, T30K, R71A, A93N, R94F) (Seq Id Nos.: 110 and 112).

The humanized light and heavy chains of the antibodies above are also interchangeable and are depicted as heavy and light chain pairs shown in Seq Id Nos.: 110 and 112 (hz Ab-AJ), Seq Id Nos.: 114 and 116 (hz Ab-AB v1; LC1 and HCl, respectively), Seq Id Nos.: 118 and 116 (hz Ab-AB v2; LC2 and HCl, respectively), Seq Id Nos.: 114 and 120 (hz Ab-AB v3; LC1 and HC2, respectively), Seq Id Nos.: 118 and 120 (hz Ab-AB v4; LC2 and HC2, respectively), Seq Id Nos.: 122 and 124 (hz Ab-AI v1; LC1 and HCl, respectively), Seq Id Nos.: 126 and 124 (hz Ab-AI v2; LC2 and HCl, respectively), Seq Id Nos.: 122 and 128 (hz Ab-AI v3; LC1 and HC2, respectively), and Seq Id Nos.: 126 and 128 (hz Ab-AI v4; LC2 and HC2, respectively) below in Table 4 with corresponding nucleic acid sequences.

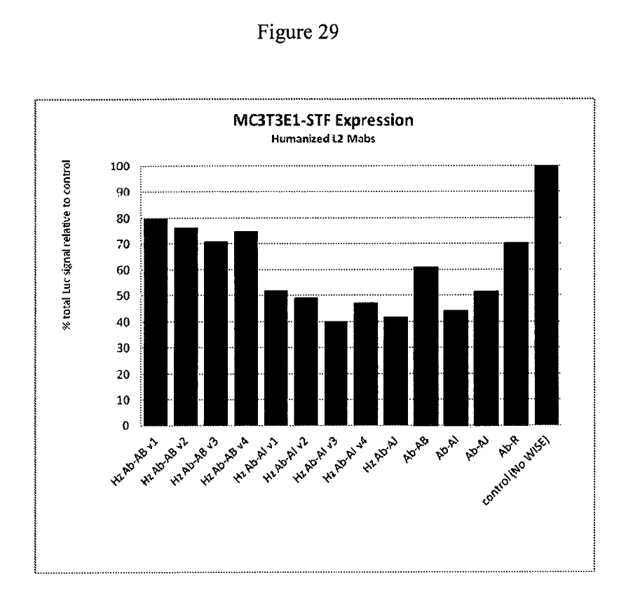

MC3T3 reporter assay using humanized antibodies to WISE is shown in FIG. 29. Ab-R is incorporated by reference from international patent application WO2009/070243. MC3T3E1-STF Expression of humanized WISE loop2 mabs: All data are presented as % total luciferase signal normalized against control. Mabs were incubated with 300 ng/ml huWISE. Reporter gene expressions were analyzed 24 hours post-incubation using luminescent substrate (Luciferase Assay System, Promega E4530) according to the manufacturer's instructions.

Isolation of D14 from Fab-310 Phage Library

Fab-310 phage library (Dyax Corp) was panned against biotinylated recombinant human WISE at 5 ug/ml, 0.5 ug/ml and 0.025 ug/ml in subsequent rounds. D14 was isolated from Round 3 overnight wash pool. 53 unique Fab phages including D14 binding to huWISE were isolated and converted to IgG2. These IgG2 molecules were tested in MC3T3-E1 functional assay. D14 showed the best inhibitory activity against huWISE.

Affinity Maturation of D14 IgG2

Affinity maturation was performed on D14 IgG2 in order to improve the inhibitory activity. Every residue in all CDRs of heavy chain (31 positions) and light chain (29 positions) was mutated by randomized mutagenesis. Seven heavy chain mutations in 5 positions and ten light chain mutations in 6 positions that improved the binding to human WISE were identified by affinity measurement on Octet QK (ForteBio) using crude condition medium samples and streptavidin Biosensors coated with biotinylated human WISE. L34, L36, H66 and H127 were four top single-residue mutants. Beneficial heavy chain mutation and light chain mutation were paired in matrix by transient transfection in 293 6E cells to yield further improved double mutants (DM, one mutation in heavy chain and one in light chain). Ten DM mutants were selected. Two best heavy chain mutations (H66 and H127) were combined by overlapping PCR. The resulted heavy chain with double mutations was paired with L34 or L36 to yield two triple mutants (TM1 and TM2).

WISE Antibody Treatment on the Progression of Renal Dysfunction Associated with Diabetic Nephropathy

The T2DN rat model is developed by combining the genomes of GK rats that develop type 2 diabetes but not progressive renal disease and FHH rats that develop progressive renal disease but not type 2 diabetes. After early-onset diabetes, overt proteinuria develops in T2DN rats at −6 months of age, and the degree of proteinuria progressively becomes more severe as the rats age. This is accompanied by hypertrophy of the glomeruli, thickening of glomerular and tubular basement membranes, expansion of the mesangial matrix, and the development of focal followed by diffuse global glomerulosclerosis and the formation of glomerular nodules by 18 months of age.

The effect of WISE antibody on the progression of renal dysfunction in this model was tested in rats of 12 month age when significant glomerular injuries have been established including glomerular hypertrophy and focal segmental glomerulosclerosis, with regional adhesion of the glomerular tuft to Bowman's capsule associated with expansion of the mesangial matrix and filling in of capillaries. At 12 months of age, glomeruli in T2DN rats also exhibit expansion of mesangial matrix and appearance of periodic acid-Schiff-positive material (reference Diabetes 53: 735-742). To accelerate the disease progression the rats underwent uninephrectomy (left kidney, which served as baseline for histology).

The proteinuria level in each individual rat was measured two weeks post surgery and right before treatment start and were used to randomize the animals into four groups: 1) naïve group, no treatment, n=10 2) Lisinopril (daily 20 mg/kg in drinking water, n=12 and served as positive control for lowering proteinuria; 3) isotype matched control IgG1 (20 mg/kg, IP injection, three times a week in antibody dilution buffer, n=12); 4) WISE antibody (20 mg/kg, IP injection, three times a week in antibody dilution buffer, n=12). Urine samples were collected at baseline and every two weeks in metabolic cages and aliquoted before testing or freezing. Serum samples were collected at baseline, week 8 post-treatment, week14 post treatment and terminal necropsy at week 16 post treatment. At necropsy, the right kidney were processed for histology (half, H&E, Masson's Trichrome) and protein/RNA (the other half). The impact on proteinuria, glomerular and interstitial fibrosis as well as renal function were evaluated.

The treatment with WISE antibody relative to that with IgG control significantly inhibited the progression of both glomerular and interstitial injuries; and rats received WISE antibody preserved and increased renal function (4%) relative to the level observed at baseline whereas rats from naïve group or from control IgG treated group had 27 and 23% decline in renal function (estimated creatinine clearance rate) relative to baseline level respectively. Thus WISE antibody is expected to have therapeutic applications for reducing renal injuries and preserve or improve renal function for diseases like diabetic nephropathy in which renal dysfunction is caused by diabetes, hypertension or a combination of both; and diseases in which renal injuries are caused by heavy proteinuria; as well as diseases in which de novo fibrosis or ongoing fibrosis leads to progressive renal or graft dysfunction, including but not limited to hypertensive kidney diseases, and transplant-related allograft fibrosis.

### Binding Profiles of WISE Loop 2 Antibodies

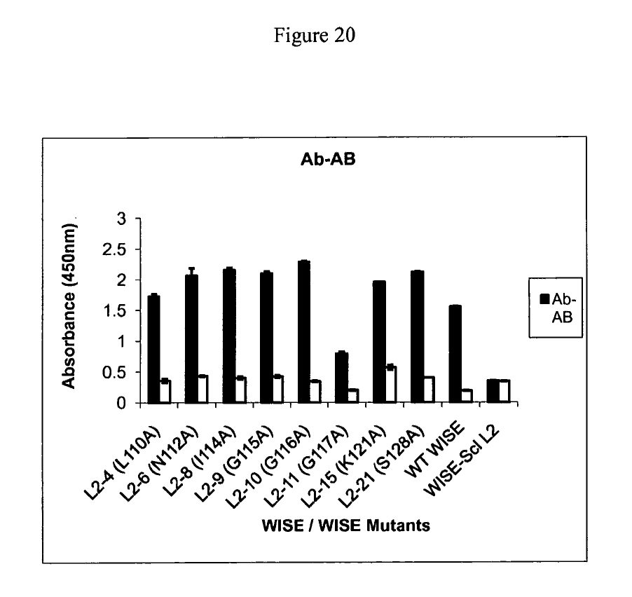

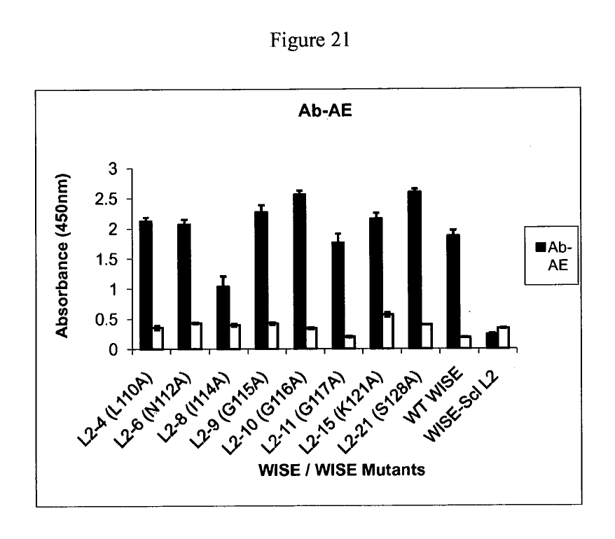

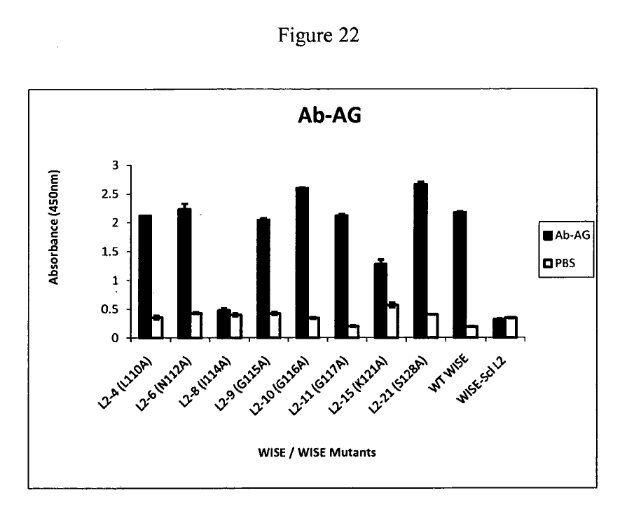

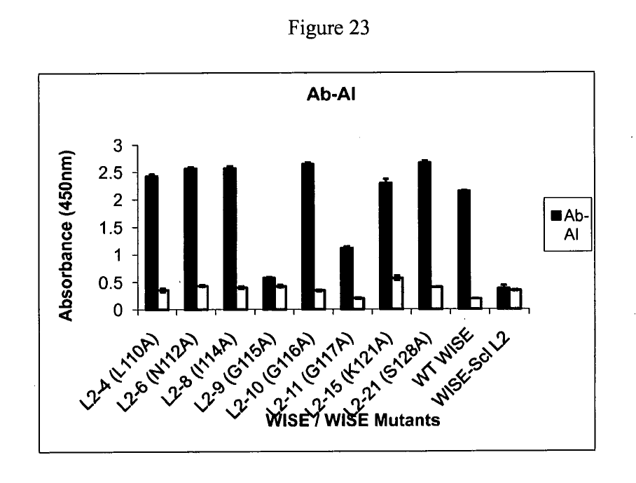

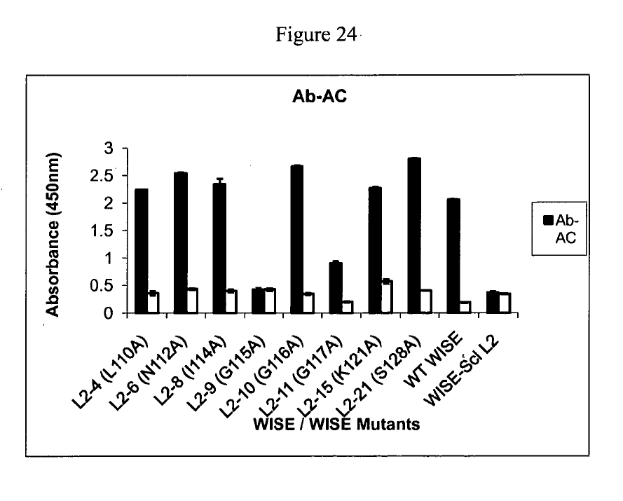

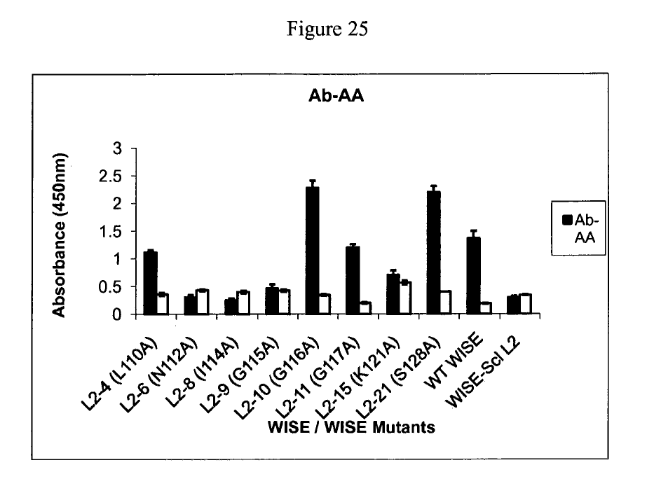

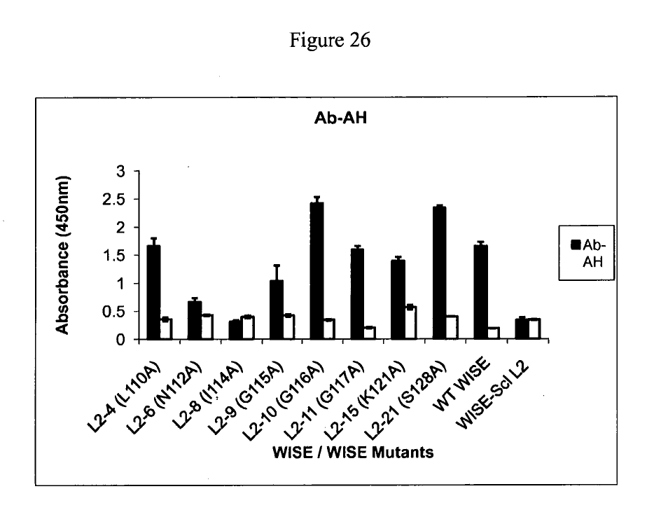

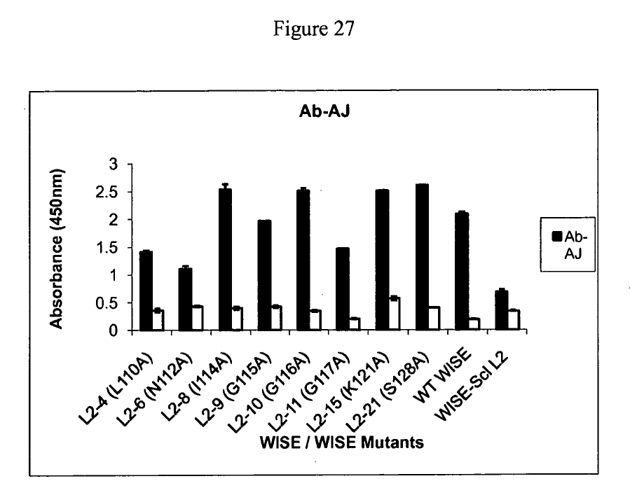

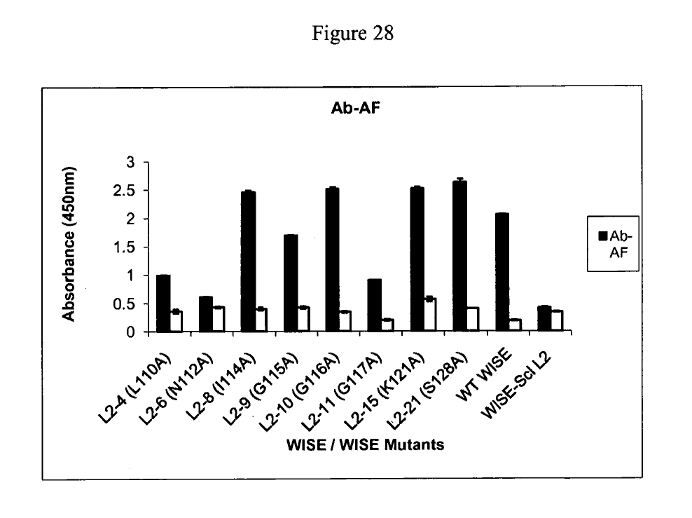

FIGS. 20-28 depict binding profiles of anti WISE antibodies that bind to the loop 2 region. FIG. 20 depicts Ab-AB against WISE mutants. FIG. 21 depicts Ab-AE against WISE mutants. FIG. 22 depicts Ab-AG against WISE mutants. FIG. 23 depicts Ab-AI. FIG. 24 depicts Ab-AC against WISE mutants. FIG. 25 depicts Ab-AA against WISE mutants. FIG. 26 depicts Ab-AH against WISE mutants. FIG. 27 depicts Ab-AJ against WISE mutants. FIG. 28 depicts Ab-AF against WISE mutants.

Mutants in FIGS. 20-28 are represented as the naturally occurring amino acid, followed by a number for the amino acid position in the WISE protein, and followed by the mutation (e.g., A=alanine). Thus, L110A is understood to represent mutation of leucine at position 110 of SEQ ID NO: 2 mutated to alanine. N112A is asparagine at position 112 of SEQ ID NO: 2 is mutated to alanine. I114A is isoleucine at position 114 of SEQ ID NO: 2 is mutated to alanine. G115A is glycine at position 115 of SEQ ID NO: 2 is mutated to alanine. G116A is glycine at position 116 of SEQ ID NO: 2 is mutated to alanine. G117A is glycine at position 117 of SEQ ID NO: 2 is mutated to alanine. K121A is lysine at position 121 of SEQ ID NO: 2 is mutated to alanine. S128A is serine at position 128 of SEQ ID NO: 2 is mutated to alanine. WISE-Scl L2 depicts the human WISE protein where the loop 2 region has been replaced with SOST loop 2. Higher absorbance represents binding while lower absorbance represents diminished binding.

Mutation of certain residues leads to diminished binding to the mutant WISE protein indicating that the antibody requires the natural occurring residue for binding. Ab-AB has diminished binding to G117A (FIG. 20). Ab-AE has diminished binding to I114A (FIG. 21). Ab-AG has diminished binding to I114A and K121A (FIG. 22). Ab-AI has diminished binding to G115A and G117A (FIG. 23). Ab-AC has diminished binding to G115A and G117A (FIG. 24). Ab-AA has diminished binding to N112A, I114A, G115A and K121A (FIG. 25). Ab-AH has diminished binding to N112A and I114A (FIG. 26). Ab-AJ has diminished binding to L11A, N112A and G117A (FIG. 27). Ab-AF has diminished binding to L11A, N112A, and G117A (FIG. 28).

Thus, an isolated antibody of the invention includes one where binding to WISE is via one or more of a leucine at amino acid 110 of SEQ ID NO: 2, an asparagine at amino acid 112 of SEQ ID NO: 2, an isoleucine at amino acid 114 of SEQ ID NO: 2, a glycine at amino acid 115 of SEQ ID NO: 2, a glycine at amino acid 116 of SEQ ID NO: 2, a glycine at amino acid 117 of SEQ ID NO: 2, a glycine at amino acid 119 of SEQ ID NO: 2, a lysine at amino acid 121 of SEQ ID NO: 2, a tryptophan at amino acid 123 of SEQ ID NO: 2, an arginine at amino acid 126 of SEQ ID NO: 2, a serine at amino acid 128 of SEQ ID NO: 2, or a glutamine at amino acid 129 of SEQ ID NO: 2. One of skill in the art will understand based on the methods described herein that one can identify antibodies that bind through or via a residue by making a mutation at the designated position which then results in reduced binding. Reduced binding is represented, for example, by Ab-AJ which binds native WISE at an absorbance of about 2. Mutation of residues L110A or N112A or G117A result in a reduction of absorbance to about 1.5 or less (FIG. 27).

While the compositions and methods of this invention have been described in terms of some embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference. Particular reference is made to antibodies to WISE in International application WO2009/070243, which is incorporated herein in it's entirety.

What is claimed is:

1. An isolated antibody or fragment thereof that cross blocks the binding of at least one of antibodies Ab-AA, Ab-AB, Ab-AC, Ab-AD, Ab-AE, Ab-AF, Ab-AG, Ab-AH, Ab-AI, Ab-AJ and D14 to human WISE and specifically binds to the loop 2 domain of human WISE and/or is cross-blocked from binding to human WISE by at least one of antibodies Ab-AA, Ab-AB, Ab-AC, Ab-AD, Ab-AE, Ab-AF, Ab-AG, Ab-AH, Ab-M, Ab-AJ and D14 and specifically binds to the loop 2 domain of human WISE.

2. The antibody or fragment thereof of claim 1 wherein said WISE antibody or fragment thereof can decrease at least one of following parameters: tissue injury and markers thereof, Sirius red staining or collagen production, expression of myofibroblast markers such as aSMA or FSP-1, osteopontin expression, proteinuria, and/or can inhibit the activity of WISE in a cell based assay.

3. The antibody of claim 1 that can increase or preserve kidney function measured by creatinine clearance rate and/or decrease the rise in serum or plasma creatinine in a patient in need thereof compared to an untreated patient.

4. An isolated antibody or fragment thereof that binds to a Loop 2 epitope of WISE.

5. The isolated antibody of claim 4, wherein said antibody binding to WISE is at one or more of a leucine at amino acid 110 of SEQ ID NO: 2, an asparagine at amino acid 112 of SEQ ID NO: 2, an isoleucine at amino acid 114 of SEQ ID NO: 2, a glycine at amino acid 115 of SEQ ID NO: 2, a glycine at amino acid 116 of SEQ ID NO: 2, a glycine at amino acid 117 of SEQ ID NO: 2, a glycine at amino acid 119 of SEQ ID NO: 2, a lysine at amino acid 121 of SEQ ID NO: 2, a tryptophan at amino acid 123 of SEQ ID NO: 2, an arginine at amino acid 126 of SEQ ID NO: 2, a serine at amino acid 128 of SEQ ID NO: 2, or a glutamine at amino acid 129 of SEQ ID NO: 2.

6. The antibody or fragment thereof of any one of claims 1-3 and 5 that comprises at least one sequence having at least 90% identity to a selected from SEQ ID NOs: 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 86, 87, 88, 89, 90, 91, 92, 93, 94, 95, 96, 97, 98, 99, 100, 101, 102, 103, 104, 105 106, 107, 108, 133, 134, 135, 136, 137, and 138 and binds to human WISE.

7. The antibody or fragment thereof of any of claims 4 and 6 comprising six of said sequences.

8. The antibody or fragment thereof according to claim 7 wherein said percent identity is 95%.

9. The antibody or fragment thereof according to claim 8 comprising:
a. sequences of SEQ ID NOs: 34, 35, and 36;
b. sequences of SEQ ID NOs: 37, 38, and 39;
c. sequences of SEQ ID NOs: 40, 41, and 42;
d. sequences of SEQ ID NOs: 43, 44, and 45;
e. sequences of SEQ ID NOs: 46, 47, and 48;
f. sequences of SEQ ID NOs: 49, 50, and 51;
g. sequences of SEQ ID NOs: 52, 53, and 54;
h. sequences of SEQ ID NOs: 55, 56, and 57;
i. sequences of SEQ ID NOs: 58, 59, and 60;
j. sequences of SEQ ID NOs: 61, 62, and 63;
k. sequences of SEQ ID NOs: 64, 65, and 66;
l. sequences of SEQ ID NOs: 67, 68, and 69;
m. sequences of SEQ ID NOs: 86, 87, and 88;
n. sequences of SEQ ID NOs: 89, 90, and 69;
o. sequences of SEQ ID NOs: 91, 92, and 93;
p. sequences of SEQ ID NOs: 94, 95, and 96;
q. sequences of SEQ ID NOs: 97, 98, and 99;
r. sequences of SEQ ID NOs: 100, 101, and 102;
s. sequences of SEQ ID NOs: 103, 104, and 105;
t. sequences of SEQ ID NOs: 106, 107, and 108;
u. sequences of SEQ ID NOs: 133, 134, and 135; and
v. sequences of SEQ ID NOs: 136, 137, and 138.

10. The antibody or fragment thereof according to claim 9 comprising:
a. sequences of SEQ ID NOs: 34, 35, and 36 and sequences of SEQ ID NOs: 37, 38, and 39;
b. sequences of SEQ ID NOs: 40, 41, and 42 and sequences of SEQ ID NOs: 43, 44, and 45;
c. sequences of SEQ ID NOs: 46, 47, and 48 and sequences of SEQ ID NOs: 49, 50, and 51;
d. sequences of SEQ ID NOs: 52, 53, and 54 and sequences of SEQ ID NOs: 55, 56, and 57;
e. sequences of SEQ ID NOs: 58, 59, and 60 and sequences of SEQ ID NOs: 61, 62, and 63;
f. sequences of SEQ ID NOs: 64, 65, and 66 and sequences of SEQ ID NOs: 67, 68, and 69;
g. sequences of SEQ ID NOs: 86, 87, and 88 and sequences of SEQ ID NOs: 89, 90, and 69;
h. sequences of SEQ ID NOs: 91, 92, and 93 and sequences of SEQ ID NOs: 94, 95, and 96;
i. sequences of SEQ ID NOs: 97, 98, and 99 and sequences of SEQ ID NOs: 100, 101, and 102;
j. sequences of SEQ ID NOs: 103, 104, and 105 and sequences of SEQ ID NOs: 106, 107, and 108; and
k. sequences of SEQ ID NOs: 133, 134, and 135 and sequences of SEQ ID NOs: 136, 137, and 138.

11. The antibody or fragment thereof according to claim 10 comprising at least one sequence having at least 90% identity to any of the sequences of a-k.

12. The antibody according to claim 10 comprising a light chain according to SEQ ID NO: 110 and a heavy chain according to SEQ ID NO: 112, a light chain according to SEQ ID NO: 114 and a heavy chain according to SEQ ID NO: 116, a light chain according to SEQ ID NO: 118 and a heavy chain according to SEQ ID NO: 116, a light chain according to SEQ ID NO: 114 and a heavy chain according to SEQ ID NO: 120, a light chain according to SEQ ID NO: 118 and a heavy chain according to SEQ ID NO: 120, a light chain according to SEQ ID NO: 122 and a heavy chain according to SEQ ID NO: 124, a light chain according to SEQ ID NO: 1126 and a heavy chain according to SEQ ID NO: 124, a light chain according to SEQ ID NO: 122 and a heavy chain according to SEQ ID NO: 128, or a light chain according to SEQ ID NO: 126 and a heavy chain according to SEQ ID NO: 128.

13. An antibody that has an affinity of at less than 1×10⁻⁷M to WISE specifically binds loop 2 of the mature polypeptide of Seq Id No: 2 and inhibits WISE activity for use in a method for treating a medical condition associated with kidney disease or disorder.

14. The antibody according to claim 13 wherein the kidney disorder is diabetic nephropathy or hypertensive renal disease or transplant-related graft dysfunction

15. The antibody according to claim 13 wherein the kidney disease is associated with proteinuria and/or fibrosis.

16. A pharmaceutical composition comprising the antibody or fragment of claim 13 or 16.

17. The antibody or fragment thereof, according to claim 16 in combination with one or more of a pharmaceutically acceptable excipient, diluent or carrier.

18. The antibody or fragment thereof according to claim 17 conjugated to at least one of Fc, PEG, albumin, and transferrin.

19. An immunogenic polypeptide of WISE suitable for use in producing inhibitory antibodies, wherein the antibodies bind to full length human WISE at an affinity of less than 1×10⁻⁷M and can decrease at least one of following parameters: tissue injury, sirius red staining or collagen production, expression of myofibroblast markers such as aSMA or FSP-1, osteopontin expression, and proteinuria, decline in renal function, and/or can block the inhibitory effect of WISE in a cell based assay.

20. A method of treating diabetic nephropathy with an antibody of claim 1.
